(19) United States
(12) Patent Application Publication
(10) Pub. No.: US 2026/0137790 A1
(43) Pub. Date: May 21, 2026

(54) MIXED VASOPRESSIN RECEPTOR AGONIST-ANTAGONIST FOR MODULATING MEAN ARTERIAL PRESSURE

(71) Applicant: FERRING B.V., Hoofddorp (NL)

(72) Inventors: Stan BUKOFZER, Redwood City, CA (US); Geoff HARRIS, Redwood City, CA (US); Hong QI, Redwood City, CA (US)

(73) Assignee: FERRING B.V., Hoofddorp (NL)

(21) Appl. No.: 19/116,676

(22) PCT Filed: Sep. 26, 2023
(86) PCT No.: PCT/US2023/075163

§ 371 Date: Mar. 28, 2025

Related U.S. Application Data
(60) Provisional application No. 63/378,010, filed on Sep. 30, 2022.
(60) Provisional application No. 63/432,974, filed on Dec. 15, 2022.
(60) Provisional application No. 63/471,712, filed on Jun. 7, 2023.

(51) Int. Cl. A61K 47/64; A61K 9/00; A61K 9/08; A61K 47/10; A61K 47/26; A61M 5/142; A61P 1/16; A61P 9/10

(52) U.S. Cl. CPC A61K 47/64; A61K 9/0019; A61K 9/08; A61K 47/10; A61K 47/26; A61M 5/14276; A61P 1/16; A61P 9/10

(57) ABSTRACT

Provided herein are compositions, such as suitable for subcutaneous administration, and methods for modulating mean arterial pressure (MAP) in an individual using a mixed V1A receptor agonist-antagonist.

### CROSS-REFERENCE

This application claims the benefit of U.S. Provisional Application No. 63/378,010, filed Sep. 30, 2022, U.S. Provisional Application No. 63/432,974, filed Dec. 15, 2022, and U.S. Provisional Application No. 63/471,712, filed Jun. 7, 2023, which are each hereby incorporated by reference in their entirety herein.

### BACKGROUND OF THE INVENTION

End-stage liver disease (ESLD) complications account for approximately 1 million deaths per year. Patients suffering from end-stage liver disease often develop portal hypertension. ESLD patients are prone to a variety of decompensating events that can compromise the perfusion pressure of the kidneys and result in progressive functional kidney injury. Hepatorenal syndrome with acute kidney injury (HRS-AKI) is a life-threatening complication and event of decompensation that develops in patients with ESLD with an untreated mortality approaching 90% (e.g., within 90 days) as well as a median survival of less than 4 weeks. If treated promptly, however, HRS-AKI is potentially reversible.

### SUMMARY OF THE INVENTION

Hepatorenal syndrome with acute kidney injury (HRS-AKI) is a serious and rapidly progressive consequence of end-stage liver disease (ESLD) that leads to acute renal failure and often death. HRS-AKI impacts up to an estimated 75,000 individuals (e.g., globally) per year and is associated with a mortality rate of more than 50 percent at 90 days following diagnosis. As the incidence of (chronic) liver disease increases, the prevalence of HRS-AKI is anticipated to also increase. Therapeutic interventions often produce only modest effects and/or fall short in achieving the goals of reversing renal failure and prolonging survival in critically-ill patients.

Management of decompensated cirrhosis often involves using vasoconstrictors that are full vasopressin 1a receptor (V1AR) agonists. Full (vasopressin V2 receptor (V2R) and V1AR) agonists reduce portal pressure by increasing splanchnic arteriolar vasoconstriction, thereby redistributing blood volume to the systemic circulation which in turn can lead to increased glomerular filtration rate and improved renal perfusion. However, (non-selective) full (V1AR, V2R) agonists (e.g., having only agonist portion and no antagonist portion) can cause undesired events, such as undesired systemic events like vasoconstriction that results in ischemia (e.g., organ ischemia and/or local (injection) site ischemia), administration (e.g., injection) site events (e.g., reactions), such as local (site) vasoconstriction that results in administration site ischemia, or both (e.g., when administered subcutaneously). Such events can preclude such compounds from being used for out-patient settings (e.g., at-home use), such as limiting their use to intravenous administration and short term applications in in-patient settings under close monitoring by specialists (e.g., thereby being unsuitable for chronic and outpatient setting). Moreover, given the risk profile of full (V2R, V1AR) agonists, careful titration and monitoring is often required to prevent the development of serious adverse events (AEs), such as those related to tissue hypoxia and ischemia resulting from excessive vasoconstriction.

Moreover, using (non-selective) full (V1AR, V2R) agonists, like vasopressin, to increase mean arterial pressure (MAP) by 10-15 mmHg, which strongly correlates with the reversal of HRS-AKI, is difficult. In some instances, achieving and/or maintaining an increase of MAP of 10-15 mmHg is a therapeutic goal of current therapies. Due to the pharmacokinetics of the vasopressin system and very steep concentration-response curve, it can be easier to either underdose (and lose clinical efficacy) or to produce too much vasoconstriction, which can lead to severe, potentially life-threatening adverse events (AEs). Secondarily, individuals with decompensated cirrhosis can already have high endogenous vasopressin levels, which facilitates water retention through the V2-mediated antidiuretic effect. Clinical vasopressin agonists are first V2 agonists, acting secondarily as V1a agonists at pharmacologic concentrations. The inherent V2 activity might contribute to the adverse event profile related to fluid overload and respiratory complications for clinical vasopressin agonists.

Provided in some embodiments herein is a compound (e.g., a mixed agonist-antagonist) having selectivity for the V1a receptor. In some embodiments, the compound reaches and maintains a (target) level of vasoconstriction and avoids fluid retention, such as through a uniform dosing profile. In some instances, the compound has a reduced incidence of (serious) adverse events and improved clinical efficacy (e.g., compared to clinical vasopressin agonists). In some instances, it is not necessary to titrate the compound, such as to achieve a reduced incidence of (serious) adverse events and improved clinical efficacy (e.g., compared to clinical vasopressin agonists). In some instances, a compound described herein (e.g., a mixed V1a agonist-antagonist) is administered at a higher dose (than necessary) and effectively reaches maximum efficacy. Contrarily, administering a comparatively high dose of a non-selective full (V1a, V2) agonist, like terlipressin, can become toxic and lead to (serious) adverse events.

In some instances, a compound described herein (e.g., a mixed V1a agonist-antagonists, such as Compound 1) is delivered (e.g., systemically) to an individual described herein after a composition comprising the compound is administered to the individual by subcutaneous administration (e.g., subcutaneous infusion or subcutaneous (bolus) injection). In some instances, a compound described herein (e.g., a mixed V1a agonist-antagonist, such as Compound 1) provides systemic effects, such as modulation of mean arterial pressure (MAP), in an individual described herein after a composition comprising the compound is administered to the individual by subcutaneous administration (e.g., subcutaneous infusion or subcutaneous (bolus) injection).

In some instances, compounds described herein (e.g., mixed V1a agonist-antagonists, such as Compound 1) are metabolized to a full vasopressin agonist, such as when administered subcutaneously. In some instances, formation of the full vasopressin agonist is associated with a higher risk of an individual developing adverse events, such as after subcutaneous (bolus) injection of the mixed V1a agonist-antagonist. In some instances, the adverse events are systemic events, local administration site events, or both. In some embodiments, the adverse events are associated with excessive vasoconstriction. In some instances, the full vasopressin agonist is partially active (compared to the mixed V1a agonist-antagonist). In some instances, administering a mixed V1a agonist-antagonist described herein subcutaneously (e.g., by subcutaneous (bolus) injection) provides an overproduction of the full vasopressin agonist (e.g., in the subcutaneous space of the individual). An overproduction of the full vasopressin agonist (e.g., in the subcutaneous space of the individual) is undesirable, for example, since less of the parent compound (e.g., a mixed V1a agonist-antagonist described herein, such as Compound 1) is delivered systemically, thereby increasing the risk of undesirable (systemic) events (e.g., toxicities associated with excessive (local and/or systemic) vasoconstriction), such as through full agonism of the V1a receptor by the full vasopressin agonist. Additional challenges arising from overproduction of the full vasopressin agonist (e.g., in the subcutaneous space of the individual) after administration of a composition described herein include reduced effectiveness, increased side-effects, and/or difficulty controlling (e.g. titrating) for efficacy. As shown in FIG. 15, full agonism of the vasopressin receptor(s) can induce serious side-effects and/or be lethal.

Described in some embodiments herein are methods and formulations for reducing formation of a full vasopressin agonist (e.g., M1), such as after subcutaneous (bolus) injection of a mixed V1a agonist-antagonist described herein (e.g., Compound 1). In some embodiments, reducing formation of a full vasopressin agonist (e.g., M1), such as after subcutaneous administration of a mixed V1a agonist-antagonist described herein, reduces the risk of an individual developing undesirable (systemic) events (e.g., toxicities associated with excessive (local and/or systemic) vasoconstriction), such as through full agonism of the V1a receptor by the full vasopressin agonist. In some embodiments, reducing formation of a full vasopressin agonist (e.g., M1), such as after subcutaneous administration of a mixed V1a agonist-antagonist described herein, increases effectiveness of a (e.g., mixed V1a agonist-antagonist) treatment described herein, decreases side-effects (e.g., associate with overproduction of the full agonist), and/or improves control (e.g. titration) of efficacy of a (e.g., mixed V1a agonist-antagonist) treatment described herein. In some embodiments, subcutaneously infusing the mixed V1a agonist-antagonist reduces metabolite (M1) formation (e.g., as measured systemically, such as by serum/plasma concentration). In some embodiments, increasing buffer concentration of a composition comprising the mixed V1a agonist-antagonist reduces metabolite (M1) formation (e.g., as measured systemically, such as by serum/plasma concentration), such as in vitro and in vivo (e.g., after subcutaneous administration). In some embodiments, increasing the concentration of the mixed V1a agonist-antagonist in the composition reduces metabolite (M1) formation (e.g., as measured systemically, such as by serum/plasma concentration), such as in vitro and in vivo (e.g., after subcutaneous administration). In some embodiments, such as when a composition described herein is administered at a relatively slow rate of administration, subcutaneously infusing and increasing the concentration of the mixed V1a agonist-antagonist in the composition reduces metabolite (M1) formation (e.g., as measured systemically, such as by serum/plasma concentration), such as in vitro and in vivo (e.g., after subcutaneous administration). In some embodiments, any combination of subcutaneous infusion, increased buffer concentration of the composition, and increased drug concentration in the composition reduces metabolite (M1) formation after subcutaneous administration (e.g., as measured systemically, such as by serum/plasma concentration). In some embodiments, subcutaneously infusing a compound described herein, increasing buffer concentration of a composition described herein, and/or increasing drug concentration in a composition describes herein improves systemic delivery of a mixed agonist-antagonist described herein.

Provided in some embodiments herein is a compound (e.g., a mixed V1AR receptor agonist-antagonist, such as Compound 1) that decreases portal pressure (PP) in an individual (e.g., in need thereof), such as after subcutaneous administration, without excessive vasoconstriction over a broad dose range, such as 10 μg/kg to 500 μg/kg.

Provided in some embodiments herein is a compound (e.g., a mixed V1a receptor agonist-antagonist, such as Compound 1) that increases mean arterial pressure (MAP) in an individual (e.g., in need thereof), such as after subcutaneous administration. In some embodiments, the increase in MAP reaches a peak plateau, such as of about +10 to +15 mmHg (e.g., even at doses as high as 100 to 500 μg/kg). In contrast, administration of a full, nonselective (V2, V1a) receptor agonist described herein, such as terlipressin, at similarly high doses provides markedly higher increases in MAP, such as well beyond a treatment window of +10 to +15 mmHg. Such large increases in MAP can significantly increase the likelihood of (serious) side effects (in the individual receiving treatment).

In some embodiments, a compound described herein (e.g., a mixed V1AR receptor agonist-antagonist, such as Compound 1) achieves a therapeutic ceiling (e.g., when administered subcutaneously), such that even after increasing the dose of the compound (e.g., to doses as high as 100 to 500 μg/kg), an effect (e.g., increasing MAP) does not (significantly) change (e.g., increase or decrease).

In some instances, increasing the dose of a compound described herein (e.g., a full, nonselective (V2R, V1AR) agonist, such as terlipressin) does provide a (significant) change (e.g., an increase) in an effect (e.g., MAP). In some instances, increasing doses of a compound described herein (e.g., a full, nonselective (V2R, V1AR) agonist, such as terlipressin) continues pushing an effect (e.g., MAP) into levels that can be detrimental and/or can lead to (severe) side effects in the individual receiving the compound.

In some instances, increasing a dose of a mixed V1a agonist-antagonist described herein, such as Compound 1, does not continue increasing MAP in an individual (e.g., even at doses as high as 100 to 500 μg/kg), whereas increasing a dose of a full, nonselective (V2, V1a) agonist, such as terlipressin, does continue increasing MAP in an individual. In some instances, mixed agonist-antagonists described herein, such as Compound 1, are safely used subcutaneously to treat ESLD or symptoms and/or complications thereof, such as without the risk of an individual developing (serious) side effects and/or having an effect, such as MAP, develop to dangerous or harmful levels. In some instances, the therapeutic window (and safety profile) of mixed V1AR agonist-antagonists described herein, such as Compound 1, is significantly improved compared to V1AR agonists (e.g., that do not comprise a discrete V1AR antagonist portion), such as terlipressin.

In some embodiments, a compound described herein has an agonist portion (e.g., D1). In some embodiments, a compound described herein has an antagonist portion (e.g., D2). In some embodiments, a compound described herein has an agonist portion (e.g., D1) and an antagonist portion (e.g., D2). In some embodiments, the antagonist portion (e.g., D2) has no (agonist) activity or substantially less (agonist) activity than the agonist portion (e.g., D1), such as having at least about 1.5× less agonist activity than the agonist portion (e.g., D1), at least about 2× less agonist activity than the agonist portion (e.g., D1), at least about 3× less agonist activity than the agonist portion (e.g., D1), 5× less agonist activity than the agonist portion (e.g., D1), at least about 10× less agonist activity than the agonist portion (e.g., D1), or at least about 100× less agonist activity than the agonist portion (e.g., D1). In some embodiments, the agonism and/or antagonism is of V1AR.

In some embodiments, compounds described herein (e.g., mixed V1a agonist-antagonists, such as Compound 1) are not full or nonselective (V2, V1a) receptor agonists. In some embodiments, (subcutaneous) administration of compounds described herein (e.g., mixed V1a agonist-antagonists, such as Compound 1) is not toxic (at therapeutic levels), e.g., even at doses as high as 100 to 500 μg/kg. In some embodiments, compounds described herein (e.g., mixed V1a agonist-antagonists, such as Compound 1) have a wide therapeutic index and are selective for the V1a receptor, such as at therapeutic doses. In some embodiments, compounds described herein (e.g., mixed V1a agonist-antagonists, such as Compound 1) are useful for subcutaneous administration. In some embodiments, compounds described herein (e.g., mixed V1a agonist-antagonists, such as Compound 1) increase mean arterial pressure (MAP) in an individual receiving one or more (subcutaneously administered) dose of the compound. In some embodiments, compounds described herein (e.g., mixed V1a agonist-antagonists, such as Compound 1) decrease portal pressure (PP) in an individual receiving one or more (subcutaneously administered) dose of the compound. In some embodiments, compounds described herein (e.g., mixed V1a agonist-antagonists, such as Compound 1) increase MAP and decrease PP in an individual receiving one or more (subcutaneously administered) dose of the compound. In some embodiments, such as after subcutaneous administration of a compound described herein (e.g., mixed V1a agonist-antagonists, such as Compound 1), a change in MAP plateaus, or reaches a therapeutic maximum, after a period of time (e.g., after about 10 minutes). In some instances, such as after subcutaneous administration of a compound described herein (e.g., a full, nonselective (V2, V1a) agonist, such as terlipressin), MAP rapidly increases and peaks after a period of time (e.g., after about 20 minutes). In some embodiments, such as after subcutaneous administration of a compound described herein (e.g., mixed V1a agonist-antagonists, such as Compound 1), a change in PP plateaus. In some embodiments, such as after subcutaneous administration of a compound described herein (e.g., mixed V1a agonist-antagonists, such as Compound 1), a change in MAP and PP plateaus or reaches a therapeutic maximum after a period of time (e.g., after about 10 minutes).

In some instances, systemic hemodynamic complications, such as portal hypertension and reflex splanchnic arteriolar vasodilation, are signs of decompensated liver cirrhosis. In some instances, splanchnic vasodilation causes blood to pool in the splanchnic circulation, fluid to leak into the abdomen and surrounding organs (ascites), and arterial pressure to drop. In some instances, such as in decompensated cirrhosis, these hemodynamic changes can lead to systemic complications, including hepatorenal syndrome-acute kidney injury (HRS-AKI).

In some instances, HRS-AKI treatment paradigms focus on re-establishing blood, portal, and splanchnic pressure to a level that will restore renal function. In some instances, treatment success is measured by raising mean arterial pressure (MAP) 10 to 20 mmHg from baseline at presentation (e.g., since this correlates with improved renal function and/or hemodynamic parameters). Unfortunately, available vasoactive agents either have limited efficacy or pose a serious risk of excessive vasoconstriction, fluid overload, or serious respiratory adverse events.

In some instances, a compound described herein (e.g., Compound 1) is a vasoconstrictor that selectively targets the vasopressin V1a molecule as a mixed agonist-antagonist. In some instances, the agonist domain of a compound described herein (e.g., Compound 1) causes desired vasoconstriction of the splanchnic vasculature (e.g., thereby reducing portal blood flow and pressure and/or improving an individual's systemic hemodynamics). In some instances, the antagonist domain of a compound described herein (e.g., Compound 1) prevents the full activation of V1a-mediated vasoconstrictive effects that drive safety concerns with other agents. In some instances, such as at therapeutic concentrations, a compound described herein (e.g., Compound 1) does not activate the vasopressin V2 receptor (e.g., which causes undesired water retention).

In some embodiments, a compound described herein (e.g., a mixed V1A agonist-antagonist, such as Compound 1) is useful for treating ESLD (or a manifestation thereof), decompensated cirrhosis, and/or complications (or symptoms) thereof, such as resistant ascites, refractory ascites, or post-paracentesis induced circulatory dysfunction.

In some instances, the mixed V1A agonist-antagonist is suitable for systemic delivery, such as provided that the mixed agonist-antagonist nature of the mixed V1A agonist-antagonist precludes (significant) injection site reactions (e.g., local vasoconstriction), such as at a subcutaneous injection site. In some instances, a mixed V1A agonist-antagonist provided herein has no (functional) vasopressin 2 (V2) receptor activity, such as at therapeutic concentrations. In some embodiments, the mixed V1A receptor agonist-antagonist is Compound 1.

In some embodiments, treating ESLD includes treating the disease itself and/or symptoms or complication associated therewith, such as ascites. In some embodiments, treating ESLD includes improving or managing quality of life, extending life, such as through treatment of symptoms and/or complications associated therewith (e.g., ascites and hepatic decompensation events).

In some instances, a mixed V1A agonist-antagonist provided herein increases mean arterial pressure (MAP). In some instances, a mixed V1A agonist-antagonist provided herein increases MAP without (significant) injection site reactions (e.g., local vasoconstriction), such as at a subcutaneous injection site.

In some instances, a mixed V1A agonist-antagonist provided herein reduces portal pressure, such as, by increasing splanchnic arteriolar vasoconstriction.

In some embodiments, a mixed V1A agonist-antagonist provided herein is used for treating complications of ESLD (e.g., cirrhotic portal hypertension), such as, HRS-AKI.

In some embodiments, a mixed V1A agonist-antagonist provided herein is used for treating ESLD (e.g., cirrhotic portal hypertension) or complications thereof, such as, HRS-AKI.

In some embodiments, Compound 1 is used for the treatment of complications of ESLD (e.g., cirrhotic portal hypertension), such as, HRS-AKI.

In some instances, a mixed V1A agonist-antagonist provided herein (e.g., Compound 1) provides a substantially improved therapeutic index (e.g., arising from a lower maximal vasoconstrictive effect and lower risk for tissue hypoxia), such as, when compared to full, non-selective (V2, VIA) receptor agonists. In some instances, a mixed V1A agonist-antagonist provided herein (e.g., Compound 1) provides about half of the maximal vasoconstriction produced by full agonists, such as, without any concomitant signs of ischemia. In some instances, a mixed V1A agonist-antagonist provided herein (e.g., Compound 1), such as when administered subcutaneously, is a (clinically) efficacious vasoconstrictor, such as having low to no local toxicity. In some instances, a mixed V1A agonist-antagonist provided herein (e.g., Compound 1) is a (clinically) efficacious vasoconstrictor (e.g., having a favorable benefit/risk profile (e.g., when administered subcutaneously), such as, having low to no local toxicity). In some instances, a mixed V1A agonist-antagonist provided herein (e.g., Compound 1) provides sufficient splanchnic vasoconstriction to lower elevated portal pressure, while minimizing risk for excessive vasoconstriction in other vascular beds with associated adverse events, such as mesenteric ischemia.

Provided in some embodiments herein is a method of modulating mean arterial pressure (MAP) in an individual (e.g., in need thereof), the method comprising subcutaneously administering to the individual (e.g., in need thereof) an effective amount of a compound, the compound being a mixed vasopressin receptor 1A (V1AR) agonist-antagonist.

Provided in some embodiments herein is a method of modulating mean arterial pressure (MAP) in an individual (e.g., in need thereof), the method comprising subcutaneously infusing into the individual (e.g., in need thereof) a composition comprising an (effective) amount of a compound, the compound being a mixed vasopressin receptor 1A (V1AR) agonist-antagonist.

Provided in some embodiments herein is a method of modulating mean arterial pressure (MAP) in an individual (e.g., in need thereof), the method comprising subcutaneously injecting into the individual (e.g., in need thereof) a composition comprising an (effective) amount of a compound, the compound being a mixed vasopressin receptor 1A (V1AR) agonist-antagonist.

In some embodiments, the mixed vasopressin receptor 1A (V1AR) agonist-antagonist is selective for V1AR over V2R. In some embodiments, the mixed vasopressin receptor 1A (V1AR) agonist-antagonist has no V2R activity, such as at therapeutic concentrations.

In some embodiments, the compound comprises a first portion having agonist activity and a second portion having antagonist activity.

In some embodiments, the modulation of mean arterial pressure (MAP) in the individual comprises raising MAP by at least 5% over baseline. In some embodiments, the modulation of mean arterial pressure (MAP) in the individual comprises raising MAP by at least 5 mmHg (e.g., 5 mmHg or more, or 10 mmHg or more) over baseline.

In some embodiments, the compound has a structure represented by Formula I:

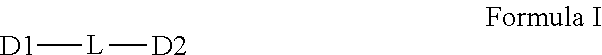

or a pharmaceutically acceptable salt thereof,

wherein:
D1 is a vasopressin receptor 1A (V1AR) agonist;
D2 is a V1AR antagonist; and
L is a linker.

Provided in some embodiments herein is a method of modulating mean arterial pressure (MAP) in an individual (e.g., in need thereof), the method comprising subcutaneously administering to the individual (e.g., in need thereof) an effective amount of a compound having a structure represented by Formula I:

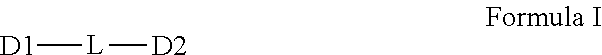

or a pharmaceutically acceptable salt thereof,

wherein:
D1 is a vasopressin receptor 1A (V1AR) agonist;
D2 is a V1AR antagonist; and
L is a linker.

Provided in some embodiments herein is a method of modulating mean arterial pressure (MAP) in an individual (e.g., in need thereof), the method comprising subcutaneously infusing into the individual (e.g., in need thereof) a composition comprising an (effective) amount of a compound having a structure represented by Formula I:

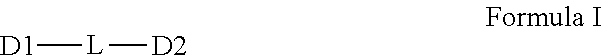

or a pharmaceutically acceptable salt thereof,

wherein:
D1 is a vasopressin receptor 1A (V1AR) agonist;
D2 is a V1AR antagonist; and
L is a linker.

In some embodiments, D1 is selective for V1AR over V2R.

In some embodiments, D1 is or comprises a (e.g., cyclic) peptide. In some embodiments, D1 is or comprises a cyclic nonapeptide. In some embodiments, D1 has or comprises the following structure:

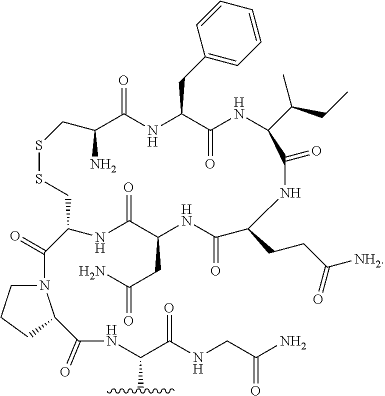 [SMILES: *[C@H](NC(=O)[C@@H]1CCCN1C(=O)[C@@H]1CSSC[C@H](N)C(=O)N[C@@H](Cc2ccccc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

In some embodiments, D1 has or comprises the following structure:

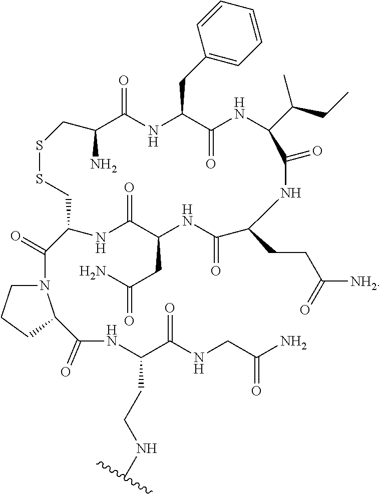 [SMILES: *NCC[C@H](NC(=O)[C@@H]1CCCN1C(=O)[C@@H]1CSSC[C@H](N)C(=O)N[C@@H](Cc2ccccc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

In some embodiments, D2 is or comprises a (e.g., linear) peptide. In some embodiments, D2 is a linear polypeptide comprising about seven or more amino acid residues.

In some embodiments, D2 has or comprises the following structure:

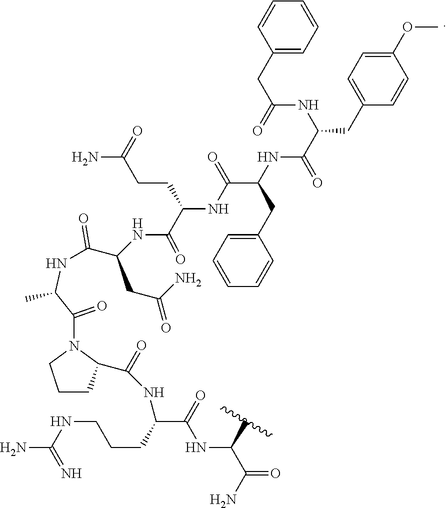 [SMILES: *[C@H](NC(=O)[C@H](CCCNC(=N)N)NC(=O)[C@@H]1CCCN1C(=O)[C@H](C)NC(=O)[C@H](CC(N)=O)NC(=O)[C@H](CCC(N)=O)NC(=O)[C@H](Cc1ccccc1)NC(=O)[C@@H](Cc1ccc(OC)cc1)NC(=O)Cc1ccccc1)C(N)=O]

In some embodiments, D2 has or comprises the following structure:

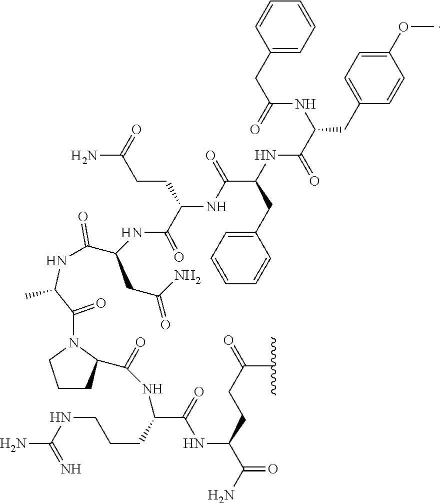 [SMILES: *C(=O)CC[C@H](NC(=O)[C@H](CCCNC(=N)N)NC(=O)[C@H]1CCCN1C(=O)[C@H](C)NC(=O)[C@H](CC(N)=O)NC(=O)[C@H](CCC(N)=O)NC(=O)[C@H](Cc1ccccc1)NC(=O)[C@@H](Cc1ccc(OC)cc1)NC(=O)Cc1ccccc1)C(N)=O]

In some embodiments, L is a non-hydrolyzable linker. In some embodiments, L comprises one or more linker group, each linker group being independently selected from the group consisting of a substituted or unsubstituted alkyl and substituted or unsubstituted heteroalkyl. In some embodiments, L is a bond, substituted or unsubstituted alkyl, or substituted or unsubstituted heteroalkyl. In some embodiments, L is or comprises substituted or unsubstituted heteroalkyl. In some embodiments, L is heteroalkyl (e.g., alkylamine) substituted with one or more substituent, each substituent being independently selected form the group consisting of oxo, amino, and substituted heteroalkyl (e.g., alkylamine substituted with oxo). In some embodiments, L is or comprises one or more (e.g., modified) amino acid residue. In some embodiments, L has or comprises the following structure:

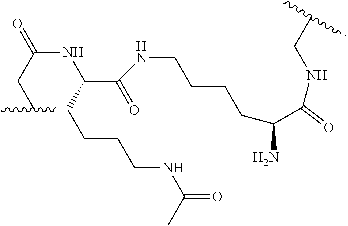 [SMILES: *CNC(=O)[C@@H](N)CCCCNC(=O)[C@H](CCCCNC(C)=O)NC(=O)C*]

In some embodiments, L has or comprises the following structure:

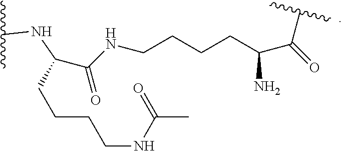 [SMILES: *N[C@@H](CCCCNC(C)=O)C(=O)NCCCC[C@H](N)C(*)=O]

In some embodiments, the compound is Compound 1, or a pharmaceutically acceptable salt thereof.

Provided in some embodiments herein is a method of modulating mean arterial pressure (MAP) in an individual (e.g., in need thereof), the method comprising subcutaneously administering to the individual (e.g., in need thereof) an effective amount of Compound 1, or a pharmaceutically acceptable salt thereof.

Provided in some embodiments herein is a method of modulating mean arterial pressure (MAP) in an individual (e.g., in need thereof), the method comprising subcutaneously infusing into the individual (e.g., in need thereof) a composition comprising an (effective) amount of Compound 1, or a pharmaceutically acceptable salt thereof.

Provided in some embodiments herein is a method of reducing (incidence of) local vasoconstriction, such as (injection site) ischemia, in an individual in need thereof, the method comprising subcutaneously infusing into the individual in need thereof a composition comprising a compound having a structure represented by Formula I, or a pharmaceutically acceptable salt thereof.

In some embodiments, the composition further comprises a liquid vehicle or solvent (e.g., water or an aqueous vehicle).

In some embodiments, the method further comprises affixing a subcutaneous infusion device to the skin of the individual, the subcutaneous infusion device comprising a chamber body and a hollow tube body, the composition being configured within the chamber body, the hollow tube body comprising a first opening and a second opening, the first opening being in fluid contact with the chamber body, and the second opening being configured subcutaneously within the individual after affixing the subcutaneous infusion device to the skin.

In some embodiments, the subcutaneous infusion device further comprises a pump configured to subcutaneously infuse the composition into the individual at a constant or varying rate.

In some embodiments, subcutaneously infusing the composition into the individual improves tolerability, relative to subcutaneous (bolus) injection (e.g., based on a reduction of M1 overproduction, such as subcutaneously). In some embodiments, subcutaneously infusing the composition into the individual reduces undesired systemic events (e.g., undesired vasoconstriction, such as resulting in ischemia), reduces undesired administration site events (e.g., local site vasoconstriction, such as resulting in administration site ischemia), or both.

Provided in some embodiments herein is a method of modulating mean arterial pressure (MAP) in an individual in need thereof, the method comprising subcutaneously infusing into the individual in need thereof a composition comprising a compound having a structure represented by Formula I, or a pharmaceutically acceptable salt thereof, wherein less than 50% of the compound of Formula I degrades (e.g., subcutaneously).

Provided in some embodiments herein is a method of modulating mean arterial pressure (MAP) in an individual in need thereof, the method comprising subcutaneously injecting into the individual in need thereof a composition comprising a compound having a structure represented by Formula I, or a pharmaceutically acceptable salt thereof, wherein less than 50% of the compound of Formula I degrades (e.g., subcutaneously).

In some embodiments, such as when the composition is subcutaneously administered to the individual (e.g., by subcutaneous (bolus) injection or subcutaneous infusion), less than 50% of the compound of Formula I degrades (e.g., subcutaneously). In some embodiments, such as when the composition is subcutaneously administered to the individual (e.g., by subcutaneous (bolus) injection or subcutaneous infusion), less than 30% of the compound of Formula I degrades (e.g., subcutaneously). In some embodiments, such as when the composition is subcutaneously administered to the individual (e.g., by subcutaneous (bolus) injection or subcutaneous infusion), less than 50% of the compound of Formula I degrades (e.g., subcutaneously) to form M1. In some embodiments, such as when the composition is subcutaneously administered to the individual (e.g., by subcutaneous (bolus) injection or subcutaneous infusion), less than 30% of the compound of Formula I degrades (e.g., subcutaneously) to form M1. In some embodiments, such as when the composition is subcutaneously infused into the individual less M1 is formed relative to administration of an otherwise identical composition administered by subcutaneous (bolus) injection. In some embodiments, such as when the composition is subcutaneously infused into the individual less M1 is formed systemically relative to administration of an otherwise identical composition administered by subcutaneous (bolus) injection. In some embodiments, such as when the composition is subcutaneously infused into the individual less M1 is formed locally (injection/infusion site) relative to administration of an otherwise identical composition administered by subcutaneous (bolus) injection.

In some embodiments, the composition is subcutaneously infused into the individual continuously for at least one hour. In some embodiments, the composition is subcutaneously infused into the individual at a rate of about 0.005 milliliters per hour (mL/hr) to about 1 mL/hr for an administration period.

In some embodiments, the compound is administered to the individual (e.g., continuously) in an amount of about 0.001 milligram (mg) to about 100 mg, such as over a period of one or more days.

In some embodiments, the composition comprises the compound in a concentration of about 0.001 milligrams per milliliters (mg/mL) to about 100 mg/mL. In some embodiments, the composition comprises the compound in a concentration of about 0.1 mg/mL to about 100 mg/mL. In some embodiments, the composition comprises the compound in a concentration of about 1 mg/mL to about 10 mg/mL.

In some embodiments, the compound is (continuously) administered to the individual in need thereof at a dose of about 0.1 mg/day to about 100 mg/day.

In some embodiments, the composition further comprises a preservative. In some embodiments, the preservative is present in an amount of about 1 mg/mL to about 20 mg/mL.

In some embodiments, the composition further comprises a solubilizing agent. In some embodiments, the solubilizing agent is present in an amount of about 1 mg/mL to about 100 mg/mL (e.g., about 60-80 mg/mL).

In some embodiments, the composition comprises a buffering agent. In some embodiments, the buffering agent is selected from the group consisting of acetate buffer, succinate buffer, and citrate buffer. In some embodiments, the composition comprises a buffering agent in a concentration of about 1 millimolar (mM) to about 1 molar (M). In some embodiments, the composition comprises a buffering agent in a concentration of about 5 mM to about 250 mM. In some embodiments, the composition comprises a buffering agent in a concentration of about 5 mM to about 25 mM. In some embodiments, the composition comprises a buffering agent in a concentration of about 50 mM to about 250 mM.

In some embodiments, the composition has a pH of about 4 to about 8. In some embodiments, the composition has a pH of about 4 to about 6. In some embodiments, the composition has a pH of about 4.5 to about 5.

In some embodiments, the mean arterial pressure (MAP) of the individual increases (e.g., compared to a baseline measurement before treatment) following subcutaneous administration of Compound 1. In some embodiments, MAP of the individual increases by about 1% to about 10% (e.g., compared to a baseline measurement before treatment) after administering a compound, or the pharmaceutically acceptable salt thereof, described herein to the individual. In some embodiments, MAP of the individual dose-dependently increases after administering a compound, or the pharmaceutically acceptable salt thereof, described herein to the individual.

In some embodiments, diastolic blood pressure of the individual increases (e.g., compared to a baseline measurement before treatment) after administering a compound, or the pharmaceutically acceptable salt thereof, described herein to the individual.

In some embodiments, systolic blood pressure of the individual increases (e.g., compared to a baseline measurement before treatment) after administering a compound, or the pharmaceutically acceptable salt thereof, described herein to the individual.

In some embodiments, the diastolic and/or systolic blood pressure of the individual dose-dependently increases after administering a compound, or the pharmaceutically acceptable salt thereof, described herein to the individual.

In some embodiments, pulse rate and/or peripheral blood flow of the individual decreases after administering a compound, or the pharmaceutically acceptable salt thereof, described herein to the individual.

In some embodiments, (subcutaneous) administration of a compound, or the pharmaceutically acceptable salt thereof, described herein to the individual improves systemic hemodynamics in the individual.

In some embodiments, (subcutaneous) administration of a compound, or the pharmaceutically acceptable salt thereof, described herein to the individual reduces fluid retention and/or overload in the individual.

In some embodiments, the method comprises administering a compound, or the pharmaceutically acceptable salt thereof, described herein to the individual on a first day and a second day (e.g., the second day being one or more days after the first day).

In some embodiments, the individual receives an initial (e.g., intravenous infusion) dose of a compound, or the pharmaceutically acceptable salt thereof, described herein on the first day (e.g., to acclimate the individual to vasoconstriction before receiving a first subcutaneous treatment dose). In some embodiments, the initial (e.g., intravenous infusion) dose is about 0.01 milligrams (mg) to about 100 mg. In some embodiments, the initial (e.g., intravenous infusion) dose is about 0.01 milligrams (mg) to about 10 mg. In some embodiments, a compound, or the pharmaceutically acceptable salt thereof, described herein is administered to the individual on the first day (e.g., by intravenous infusion) for a period of about 4 hr to about 8 hr (e.g., about 6 hr). In some embodiments, the initial (e.g., intravenous infusion) dose of a compound, or the pharmaceutically acceptable salt thereof, described herein is a low dose, such as a dose of about 5 μg/hr to about 15 μg/hr (e.g., about 8 μg/hr). In some embodiments, the method further comprises (subcutaneously) administering a compound, or the pharmaceutically acceptable salt thereof, described herein to the individual on one or more day after the first day. In some embodiments, the method comprises (subcutaneously) administering a compound, or the pharmaceutically acceptable salt thereof, described herein to the individual daily for two or more days (e.g., 4 to 10 days) (e.g., after the first day). In some embodiments, the method further comprises (subcutaneously) administering a compound, or the pharmaceutically acceptable salt thereof, described herein to the individual on consecutive days after the first day. In some embodiments, the method comprises (subcutaneously) administering a compound, or the pharmaceutically acceptable salt thereof, described herein to the individual on multiple days. In some embodiments, the individual receives repeated subcutaneous injections of the compound, or the pharmaceutically acceptable salt thereof, described herein.

In some embodiments, the individual is subcutaneously administered the compound, or the pharmaceutically acceptable salt thereof, described herein multiple times, such as over the course of numerous days. In some embodiments, the compound is continuously administered to the individual, such as over a period of numerous days.

In some embodiments, the method comprises subcutaneously administering the compound, or the pharmaceutically acceptable salt thereof, described herein to the individual once-daily (e.g., for two or more (e.g., five or more) consecutive days).

In some embodiments, the method comprises administering the compound, or the pharmaceutically acceptable salt thereof, described herein to the individual by subcutaneous (bolus) injection, such as where the compound is administered to the individual as a single dose (e.g., all at once).

In some embodiments, the method comprises administering the compound, or the pharmaceutically acceptable salt thereof, described herein to the individual by subcutaneous infusion. In some embodiments, the method comprises administering the compound, or the pharmaceutically acceptable salt thereof, described herein to the individual by continuous subcutaneous infusion.

In some embodiments, the method comprises (e.g., subcutaneously) administering a compound, or the pharmaceutically acceptable salt thereof, described herein to the individual in an amount of about 0.01 milligrams (mg)/day to about 100 mg/day (e.g., about 0.01 milligrams (mg)/day to about 10 mg/day (e.g., about 0.01 mg/day to about 1 mg/day)).

In some embodiments, the individual has hepatorenal syndrome with HRS-AKI.

In some embodiments, the individual has end-stage liver disease (ESLD).

In some embodiments, the individual has developed HRS-AKI as a complication of ESLD.

In some embodiments, the method further comprises reducing serum creatinine (sCr) (value) in the individual (e.g., compared to a baseline measurement before treatment). In some embodiments, the method comprises administering a compound, or the pharmaceutically acceptable salt thereof, described herein to the individual at least until the individual has a sCr value of 1.5 milligrams (mg)/deciliters (dL) or less. In some embodiments, a compound, or the pharmaceutically acceptable salt thereof, described herein (e.g., Compound 1) is (e.g., subcutaneously) administered to an individual (e.g., described herein) at least until the sCr value of the individual returns to normal (e.g., baseline).

Provided in some embodiments herein is a pharmaceutical composition comprising an effective amount of a compound, or a pharmaceutically acceptable salt thereof, wherein the compound is a mixed vasopressin receptor 1A (V1AR) agonist-antagonist, the composition being formulated for subcutaneous administration.

In some embodiments, the compound has a structure represented by Formula I:

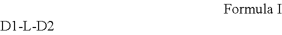

or a pharmaceutically acceptable salt thereof,

wherein:
D1 is a vasopressin receptor 1A (V1AR) agonist;
D2 is a V1AR antagonist; and
L is a linker.

Provided in some embodiments herein is a pharmaceutical composition comprising an effective amount of a compound having a structure represented by Formula I:

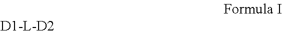

or a pharmaceutically acceptable salt thereof,

wherein:
D1 is a vasopressin receptor 1A (V1AR) agonist;
D2 is a V1AR antagonist; and
L is a linker,

the composition being formulated for subcutaneous administration.

In some embodiments, pharmaceutical composition comprising an effective amount of Compound 1, or a pharmaceutically acceptable salt thereof, the composition being formulated for subcutaneous administration.

In some embodiments, the composition is suitable for routes of administration beyond intravenous administration, such as subcutaneous administration.

In some instances, a mixed V1A agonist-antagonist provided herein (e.g., Compound 1) is administered in a form described in Example 1.

Provided in some embodiments pharmaceutical composition comprising an effective amount of Compound 1, or a pharmaceutically acceptable salt thereof, the composition being formulated for subcutaneous administration.

Provided in some embodiments herein is a subcutaneous formulation comprising a compound having a structure represented by Formula I, or a pharmaceutically acceptable salt thereof, wherein less than 50% of the compound of Formula I degrades (e.g., subcutaneously).

Provided in some embodiments herein is a subcutaneous formulation comprising a compound having a structure represented by Formula I, or a pharmaceutically acceptable salt thereof, the formulation having a concentration of the compound of Formula I of about 0.1 mg/mL to about 100 mg/mL.

Provided in some embodiments herein is a subcutaneous formulation comprising a compound having a structure represented by Formula I, or a pharmaceutically acceptable salt thereof, and a buffering agent at a concentration of about 1 millimolar (mM) to about 1 M.

In some embodiments, the formulation comprises a buffering agent at a concentration of about 1 millimolar (mM) to about 1M. In some embodiments, the buffering agent has a pKa of about 3.0 to about 6.0, such as at 25° C. In some embodiments, the buffering agent is selected from the group consisting of acetate, citrate, succinate, and phosphate.

In some embodiments, the formulation comprises a pH sufficient to inhibit (e.g., deactivate or inactivate) a protease (e.g., trypsin), such as in subcutaneous layer of an individual subcutaneously administered the formulation. In some embodiments, the pH of the formulation is about 4 to about 5 (e.g., about 4.5).

In some embodiments, the pH of the subcutaneous formulation does not (substantially) change when administered subcutaneously to an individual (e.g., by subcutaneous (bolus) injection or subcutaneous infusion).

In some embodiments, the formulation has an ionic strength of about 5 mM to about 200 mM (e.g., about 10 mM to about 100 mM).

In some embodiments, the subcutaneous formulation further comprising a preservative.

Provided in some embodiments herein is a subcutaneous formulation comprising a compound having a structure represented by Formula I, or a pharmaceutically acceptable salt thereof, and a preservative.

In some embodiments, the preservative is any suitable preservative, such as meta (m)-cresol. In some embodiments, the formulation comprises the preservative (e.g., m-cresol) at a concentration of about 1 mg/mL to about 100 mg/mL.

Provided in some embodiments herein is a subcutaneous formulation comprising a compound having a structure represented by Formula I, or a pharmaceutically acceptable salt thereof, and a solubilizing agent.

In some embodiments, the preservative is any suitable solubilizing agent, such as a cyclodextrin. In some embodiments, the formulation comprises the solubilizing agent (e.g., cyclodextrin) at a concentration of about 1 mg/mL to about 100 mg/mL (e.g., about 60-80 mg/mL).

In some embodiments, the compound of Formula I is less susceptible to degradation, such as in the subcutaneous layer of an individual subcutaneously administered the formulation.

In some embodiments, less than 50% of the compound of Formula I degrades (e.g., in a vial and/or subcutaneously), such as over a period of about one or more day (e.g., about one day, about two days, or more).

In some embodiments, the compound of Formula I is present in the formulation at a concentration of about 0.1 mg/mL to about 100 mg/mL. In some embodiments, the compound of Formula I is present in the formulation at a concentration of about 1 mg/mL to about 50 mg/mL.

In some embodiments, the compound is Compound 1.

In some embodiments, the composition is suitable for systemic delivery of an active agent, such as Compound 1.

In some instances, a mixed V1A agonist-antagonist provided herein (e.g., Compound 1) is administered in the form of an acetate salt.

In some instances, a mixed V1A agonist-antagonist provided herein (e.g., Compound 1) is administered in a form described in any example described herein, such as any one of Examples 1-6.

Provided in some embodiments herein is system for modulating mean arterial pressure (MAP), the system comprising a composition comprising a compound of Formula I, or a pharmaceutically acceptable salt thereof; and a device configured to provide subcutaneous infusion of the composition to an individual when the device is positioned on the skin of the individual.

In some embodiments, the system comprises an adhesive body for (e.g., reversibly) affixing the (subcutaneous infusion) device to the surface of the skin of the individual. In some embodiments, the system comprises a chamber body and a hollow tube body, the composition being configured within the chamber body. In some embodiments, the hollow tube body comprises a first opening and a second opening. In some embodiments, the first opening is in fluid contact with the chamber body. In some embodiments, the second opening is configured subcutaneously within the individual after affixing the subcutaneous infusion device to the skin of the individual.

In some embodiments, the (subcutaneous infusion) device further comprises a pump configured to subcutaneously infuse the composition into the individual at a constant or varying rate.

In some embodiments, the system is configured to (continuously) provide the composition to the individual over a period of about 24 hours or more.

In some embodiments, the device is configured to receive a vial and/or a cartridge of the composition.

In some embodiments, the device is a subcutaneous infusion device (e.g., pump).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates the study design described in Example 1 for each subject in Period 1 (intravenous infusion) and Period 2 (subcutaneous injection).

FIG. 2 shows a time course of Compound 1 concentration after intravenous administration, Period 1.

FIG. 3A shows a time course of Compound 1 concentration after the 1st subcutaneous administration, Period 2.

FIG. 3B shows a time course of Compound 1 concentration after the 5th subcutaneous administration, Period 2.

FIG. 4A shows diastolic blood pressure, intravenous infusion (mean values).

FIG. 4B shows diastolic blood pressure, intravenous infusion (mean percent change).

FIG. 5A shows diastolic blood pressure, repeated subcutaneous injections (mean values).

FIG. 5B shows diastolic blood pressure, repeated subcutaneous injections (mean percent change).

FIG. 6A shows systolic blood pressure, intravenous infusion (mean values).

FIG. 6B shows systolic blood pressure, intravenous infusion (mean percent change).

FIG. 7A shows systolic blood pressure, subcutaneous injections (mean values).

FIG. 7B shows systolic blood pressure, subcutaneous injections (mean percent change).

FIG. 8A shows change in mean arterial pressure over time (mean percentage change from baseline in mean arterial pressure after intravenous infusion).

FIG. 8B shows change in mean arterial pressure over time (mean percentage change from baseline in mean arterial pressure after repeated subcutaneous injections).

FIG. 9A shows pulse rate after intravenous infusion (mean values).

FIG. 9B shows pulse rate after intravenous infusion (mean percent change).

FIG. 10A shows pulse rate after subcutaneous injections (mean values).

FIG. 10B shows pulse rate after subcutaneous injections (mean percent change).

FIG. 11 shows amount of Compound 1 excreted intact in urine after intravenous infusion.

FIG. 12A shows amount Compound 1 excreted after 1st subcutaneous injection.

FIG. 12B shows amount Compound 1 excreted after 5th subcutaneous injection.

FIG. 13A shows mean plasma concentration of metabolite M1 after 1st s.c. injection of Compound 1.

FIG. 13B shows mean plasma concentration of metabolite M1 after 5th s.c. injection of Compound 1.

FIG. 14 shows the structure of M1 (encircled) in relation to Compound 1.

FIG. 15 illustrates exemplary dose-response curves for full agonists, partial agonists, and weak agonists. FIG. 15 generally illustrates that a broader therapeutic window for vasoconstriction can be achieved with curve 2 than curves 1 or 3. Portion A illustrates levels of full agonists where vasoconstriction can be lethal. Portion A illustrates levels of full agonists where vasoconstriction can induce serious side-effects.

FIG. 16 illustrates the change in portal pressure (APP) over time in rats with bile duct ligation (BDL) following subcutaneous administration of various doses of a mixed V1a agonist-antagonist.

FIG. 17 illustrates the change in portal mean arterial pressure (AMAP) over time in methionine/choline-deficient (MCD) diet rats following subcutaneous administration of a full, non-selective (V2, V1a) agonist and various doses of a mixed V1a agonist-antagonist.

FIG. 18 illustrates the change in portal pressure (APP) over time in methionine/choline-deficient (MCD) diet rats following subcutaneous administration of a full, non-selective (V2, V1a) agonist and various doses of a mixed V1a agonist-antagonist.

FIG. 19 illustrates an exemplary dose response curve of maximal possible effect at human V1a (hV1a) receptors for a full, nonselective (V2, V1a) agonist and a mixed V1a agonist-antagonist.

FIG. 20 illustrates an exemplary dose response curve of maximal possible effect at human V1a (hV1a) receptors and human V2 (hV2) receptors for a mixed V1a agonist-antagonist.

FIG. 21 illustrates an exemplary dose response curve for contractility of human mesenteric resistance arteries in response to a mixed V1a agonist-antagonist.

FIG. 22A shows a normalized plasma concentration time profile of Compound 1 after IV administration an individual (e.g., a mammal) (10 mg/kg).

FIG. 22B shows a normalized plasma concentration time profile of Compound 1 after subcutaneous administration in an individual (e.g., a mammal) (1.0 mg/kg).

FIG. 23A shows a dose response of Compound 1, vasopressin, and the vehicle on skin blood flow (SBF) (% baseline) in an individual (e.g., a mammal) after IV administration.

FIG. 23B shows a dose response of Compound 1, vasopressin, and the vehicle on blood lactate concentration (mM) in an individual (e.g., a mammal) after IV administration.

FIG. 23C shows a comparison of blood lactate concentration in an individual (e.g., a mammal) after administration of vehicle, Compound 1, or vasopressin (AVP).

FIG. 24A shows mean arterial pressure (MAP) following subcutaneous administration of Compound 1 in an individual (e.g., a mammal).

FIG. 24B shows systolic arterial blood pressure following subcutaneous administration of Compound 1 in an individual (e.g., a mammal).

FIG. 24C shows diastolic arterial blood pressure following subcutaneous administration of Compound 1 in an individual (e.g., a mammal).

FIG. 24D shows heart rate following subcutaneous administration of Compound 1 in an individual (e.g., a mammal).

FIG. 25A shows a time profile of normalized plasma concentration (ng/mL) of Compound 1 following IV bolus (0.05 mg/kg) administration in an individual (e.g., a mammal).

FIG. 25B shows a time profile of normalized plasma concentration of Compound 1 following subcutaneous bolus (0.5 mg/kg) administration in an individual (e.g., a mammal).

FIG. 26A shows a change from baseline in mean arterial pressure (AMAP) following administration of Compound 1 over 480 minutes in an individual (e.g., a mammal).

FIG. 26B shows a change from baseline in mean arterial pressure (AMAP) following administration of terlipressin over 480 minutes in an individual (e.g., a mammal).

FIG. 27 shows that little to no metabolism of Compound 1 occurs in healthy humans after intravenous (IV) infusion (panel A), and the concentration of Compound 1 and M1 are about equimolar in healthy humans after subcutaneous (bolus) injection (panel B).

FIG. 28 shows that little to no metabolism of Compound 1 occurs in mini-pigs after subcutaneous (SC) infusion of relatively low (panels A and B) and high (panels C and D) doses of Compound 1.

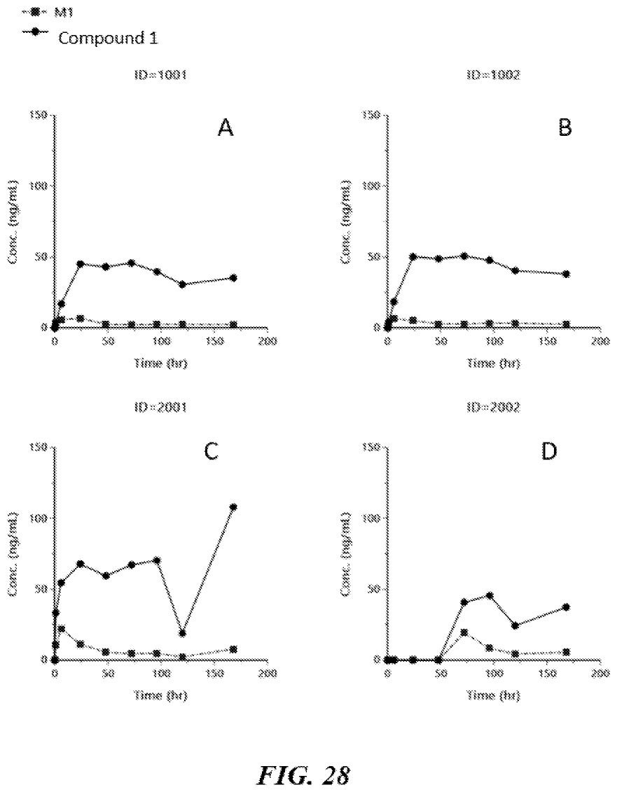

FIG. 29 depicts the Compound 1/M1 ratio of FIG. 28, panels A and B.

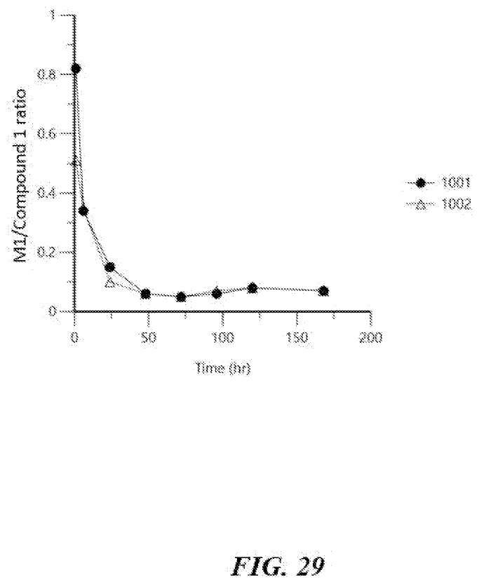

FIG. 30 shows that little to no metabolism of Compound 1 occurs in mini-pigs after relatively slow subcutaneous (SC) infusion of relatively low (panels A and B) and high (panels C and D) doses of Compound 1.

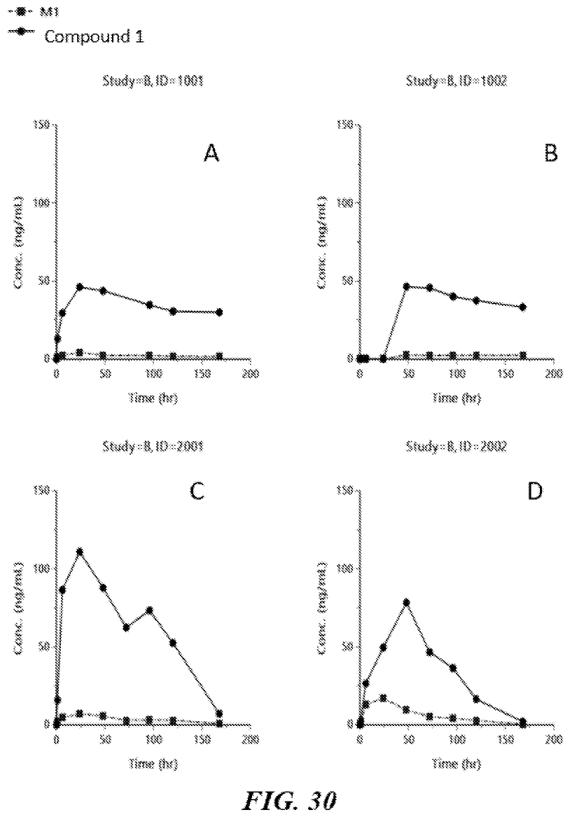

FIG. 31 depicts the Compound 1/M1 ratios of FIG. 30, panels A-D.

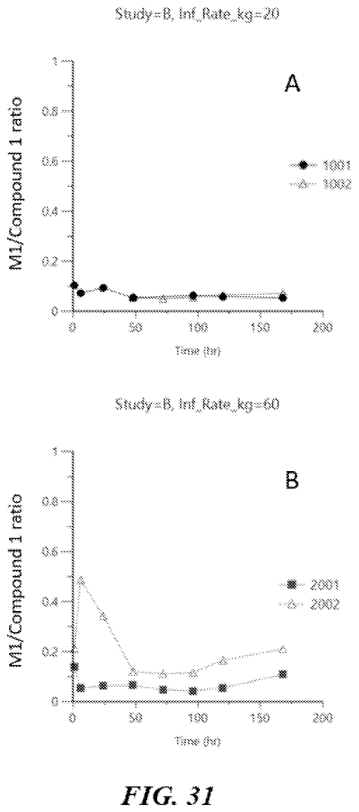

### DETAILED DESCRIPTION OF THE INVENTION

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. Any recitation of “about” provided herein also includes disclosure of the number itself. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

The terms “modulate” or “modulating” as used herein, refer to a change in a biological, chemical, and/or biochemical response, such as a physiological response, in an individual. In some instances, the change is an increase in the biological, chemical, and/or biochemical response of individual. In some instances, the change is a decrease in the biological, chemical, and/or biochemical response of individual. In some instances, the change is observed (e.g., immediately, 30 mins or more, 1 hour or more, 6 hours or more, 12 hours or more, 24 hours or more, or 1 week or more) after a compound described herein is administered to the individual.

The terms “adverse event” or “AE” as used herein, refer to an untoward medical occurrence in an individual, such as an individual participating in a clinical trial. In some instances, an AE is an unfavorable and/or unintended sign, symptom, or disease, such as being temporally associated with the use of an investigative medicinal product (IMP), whether or not considered to be caused by the IMP. For example, an AE can include accidental injuries, reasons for any change in medication (drug and/or dose), reasons for any medical, nursing or pharmacy consultation, or reasons for admission to hospital or surgical procedures, and overdoses and medication errors with or without clinical consequences. In some instances, AEs are anticipated based on the pharmacological effect of the IMP. In some instances, an AE is a laboratory abnormality, vital sign or finding from physical or gynecological examination assessed as clinically significant by the investigator. In some instances, a pre-treatment adverse event is any untoward medical occurrence arising or observed between signing of informed consent and the first administration of the IMP. In some instances, a treatment emergent adverse event is an AE occurring after the administration of the IMP and within the time of residual drug effect, or a pre-treatment adverse event or pre-existing medical condition that worsens in intensity after administration of the IMP and within the time of residual drug effect. In some instances, the time of residual drug effect is the estimated period of time after the administration of the IMP, where the effect of the product is still considered to be present based on PK, PD or other substance characteristics. In some instances, the residual drug effect is 5 times the terminal half-life. In some instances, the terminal half-life of Compound 1 is about 1.5-2 hours. In some instances, the residual drug effect is within the time to the last assessment in Period 1 and the follow-up visit in Period 2 (as described in the examples hereinbelow). In some instances, a post-treatment emergent adverse event is an AE occurring after the time of residual drug effect of the IMP (e.g., prior to the first administration in Period 2 after the last assessment in Period 1, and after the follow-up visit in Period 2).

“Amino” refers to the —NH₂ radical.

“Cyano” refers to the —CN radical.

“Nitro” refers to the —NO₂ radical.

“Oxo” refers to the ═O radical.

“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl).

In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a}(where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkenyl” refers to a radical of the formula —R^{c}-carbocyclyl where R^{c} is an alkenylene chain as defined above. The alkenylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁s heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hickel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

In general, optionally substituted groups are each independently substituted or unsubstituted. Each recitation of a optionally substituted group provided herein, unless otherwise stated, includes an independent and explicit recitation of both an unsubstituted group and a substituted group (e.g., substituted in certain embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

“Pharmaceutically acceptable salt” includes both acid and base addition salts. A pharmaceutically acceptable salt of any one of the pharmacological agents described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Systemic hemodynamic complications can be indicative of cirrhosis and portal hypertension (PHT). Individuals can often develop elevations of portal pressure (PP) due to an increase in intrahepatic resistance. The combination of increased blood flow and elevated intrahepatic resistance can lead to the development of PHT and the common manifestations of decompensated cirrhosis. Clinical PHT can occur when the hepatic venous pressure gradient (HVPG) is >5 mmHg. PHT can then lead to a hyperdynamic state characterized by a decrease in splanchnic and systemic vascular resistance, which can further increase portal blood flow. As advanced cirrhosis progresses, splanchnic arteriolar vasodilation can worsen PHT, which can then lead to further increases in the HVPG, further leading to hypoperfusion of the kidneys at 10 mmHg or above. The kidney can sense low perfusion pressure and reduced glomerular filtration rate as hypovolemia, which can then activate the renin-angiotensin-aldosterone (RAAS) and vasopressin systems, leading to severe vasoconstriction within the kidney and retention of sodium and water. If hypoperfusion of the kidneys is severe enough, it can result in the development of ascites and renal injury in the form of hepatorenal syndrome-acute kidney injury (HRS-AKI).

Management of HRS-AKI often focuses on restoring systemic arterial blood pressure and reducing PHT through splanchnic vasoconstriction, with the vasopressin system being a target, and a long-term goal being liver transplantation. While challenging to monitor and titrate, increases in vasoconstriction (i.e., mean arterial pressure (MAP)) have been correlated with improved hemodynamic parameters. Vasoconstrictive agents, including terlipressin, norepinephrine, and midodrine/octreotide, have been used as therapy for patients with HRS-AKI in an attempt to restore renal perfusion and function. Terlipressin (a vasopressin analog) plus albumin has been used as first-line therapy for HRS-AKI, as it reduces short-term mortality compared with placebo. Albumin is added to increase circulating volume. Terlipressin is approved by the US Food and Drug Administration for the treatment of adults with HRS with rapid reduction in kidney function.

There are three vasopressin receptors: V1a, V1b, and V2. V1a receptors are found throughout the circulatory system and modulate vasoconstriction. The V2 receptors modulate aquaresis through an antidiuretic effect at the level of the kidney by mediating water reabsorption in collecting tubules. V1b receptors are found in the anterior pituitary and peripheral tissues, and one potential role is to mediate the release of adrenocorticotropin hormone, which can stimulate water retention. Vasopressin, also known as arginine vasopressin (AVP) or antidiuretic hormone, is a peptide involved in water balance and vascular tone. At normal physiologic concentrations, there is little to no activity on the V1a system; only at supraphysiologic concentrations is there meaningful vasoconstriction. With pharmacologic application, intense vasoconstriction can be achieved in a concentration-dependent manner.

Arginine vasopressin (AVP) is the endogenous ligand of the vasopressin V1A, V1B, and V2 G-protein-coupled receptors (V1AR, V1BR, V2R). Homeostatic functions of the vasopressin system, such as regulation of blood osmolality and pressor effects, are mediated by the V2 and V1A receptor subtypes. Activation of V2 receptors located in kidney collecting ducts plays a role in the regulation of fluid balance through antidiuretic action. Activation of V1A receptors located on vascular smooth muscle cells provides vasoconstriction and increased arterial pressure.

Lysine vasopressin (LVP), the active metabolite of terlipressin, has activity at V1a, V1b, and V2 receptors and is a full agonist. Despite significantly improving renal function, terlipressin use is associated with serious adverse events, including gastrointestinal disorders, sepsis, and respiratory failure. These adverse effects, which can be attributed to the strength of LVP binding to V1a and off-target effects on V2, may lead to further water retention. As a result of the potential for serious side effects, terlipressin carries a black box warning from the US FDA for serious or fatal respiratory failure.

The pressor activity of vasopressin receptor agonists is of clinical interest, as demonstrated by the use of AVP and its analogs (e.g., terlipressin and ornithine vasopressin). However, a significant drawback of existing V1A receptor full agonists is the potential to induce severe vasoconstriction and tissue hypoperfusion when used at therapeutic doses. The pharmacological activity of V1AR partial agonists (e.g., compounds that have a reduced maximal efficacy at the V1A receptor) can be used in a variety of conditions where a modest increase in blood flow and/or blood pressure without hypoperfusion is desirable. For example, such indications could include hepatorenal syndrome, refractory ascites, bleeding esophageal varices, anesthesia-induced hypotension, vasodilatory shock, paracentesis-induced circulatory dysfunction, and spontaneous bacterial peritonitis.

Individuals with liver cirrhosis often develop numerous clinical complications, among which ascites accumulation is paramount and indicates poor prognosis. In some instances, ascites formation results from the homeostatic activation of endogenous sodium and water retaining systems in attempt to counteract the circulatory dysfunction, such as developed in patients with advanced liver disease. In some instances, a feature of the circulatory dysfunction is the existence of splanchnic vasodilation and portal hypertension. In some embodiments, a compound described herein (e.g., Compound 1) has an effect on ascites volume, sodium and water excretion, portal hypertension, and systemic hemodynamics following administration of the compound or vehicle to cirrhotic rats with ascites.

In some embodiments, a compound described herein (e.g., a mixed V1a agonist/antagonist, such as Compound 1) is systemically delivered in an individual after intravenous (IV) and/or subcutaneous (SC) administration of a composition comprising the compound (see Example 1). In some embodiments, the individual has systemic effects, such as a change in MAP, after the compound is administered intravenously and/or subcutaneously. In some embodiments, the compound is administered by intravenous infusion. In some embodiments, the compound is administered by subcutaneous (bolus) injection. While the systemic effects observed after IV infusion and SC (bolus) injection were comparable, more adverse events were measured in individuals receiving SC (bolus) injection of the composition (see Example 1). Generally, the compound was well-tolerated by individuals receiving the composition by IV infusion.

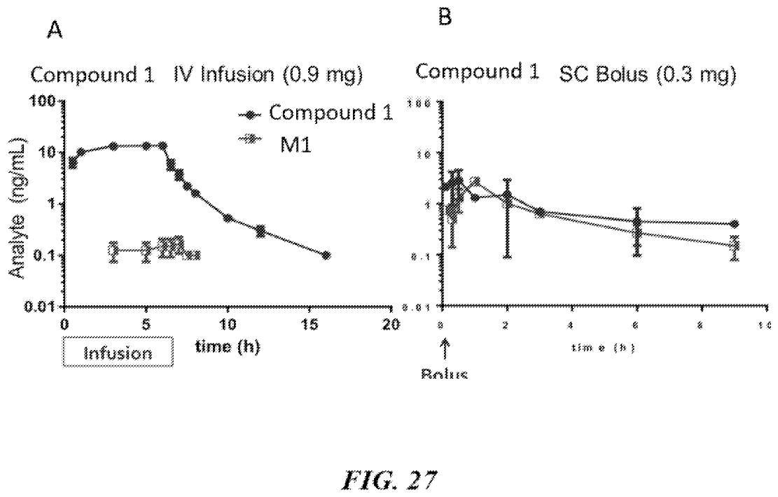

In some instances, a metabolite (e.g., a substantial amount of the metabolite M1 (e.g., an overproduction of M1)) forms after SC (bolus) injection of a composition comprising a compound described herein (e.g., a mixed V1a agonist/antagonist, such as Compound 1) (see FIG. 27, panel B). In some instances, minimal amounts of the metabolite (e.g., less than 15% of the metabolite M1) forms after IV infusion of a composition comprising a compound described herein (e.g., a mixed V1a agonist/antagonist, such as Compound 1) (see FIG. 27, panel A). As discussed herein, full vasopressin receptor agonists described herein (e.g., terlipressin) are known to cause (serious) adverse events when administered subcutaneously. As such, the overproduction of a full agonist (M1) after SC (bolus) injection provides an explanation for the differences between the tolerability profile of the composition in healthy individuals after IV infusion and (SC) bolus injection.

As described herein, in some instances, metabolite (M1) formation is reduced by subcutaneously infusing a composition comprising a compound described herein into an individual (see Example 3). Additionally, in some instances, metabolite (M1) formation is reduced by increasing buffer concentration of a composition comprising a compound described herein (see Example 4). Moreover, in some instances, metabolite (M1) formation is reduced by increasing parent drug (e.g., Compound 1) concentration of a composition comprising a compound described herein (see Example 4). In some instances, metabolite (M1) formation is reduced through a combination of any one or more of subcutaneous infusion, increased buffer concentration, and increased drug concentration.

Provided in some embodiments herein is a method of modulating mean arterial pressure (MAP) in an individual in need thereof, the method comprising subcutaneously infusing into the individual in need thereof a composition comprising a compound described herein, such as a compound having a structure represented by Formula I, or a pharmaceutically acceptable salt thereof. In some embodiments, less than 50% of the compound, such as the compound of Formula I, degrades. In some embodiments, less than 50% of the compound, such as the compound of Formula I, degrades subcutaneously.

In some embodiments, a composition described herein is subcutaneously administered to the individual and less than 50% of the compound of Formula I degrades (e.g., subcutaneously). In some embodiments, a composition described herein is subcutaneously administered to the individual by subcutaneous (bolus) injection and less than 50% of the compound of Formula I degrades (e.g., subcutaneously). In some embodiments, a composition described herein is subcutaneously administered to the individual by subcutaneous infusion and less than 50% of the compound of Formula I degrades (e.g., subcutaneously). In some embodiments, the compound of Formula I degrades subcutaneously to form M1.

In some embodiments, such as after subcutaneous (bolus) injection, more than 50% of the parent compound (e.g., Compound 1) degrades (e.g., to form M1). In some embodiments, such as after subcutaneous (bolus) injection, more than 60% of the parent compound (e.g., Compound 1) degrades (e.g., to form M1). In some embodiments, such as after subcutaneous (bolus) injection, more than 70% of the parent compound (e.g., Compound 1) degrades (e.g., to form M1). In some embodiments, such as after subcutaneous (bolus) injection, more than 80% of the parent compound (e.g., Compound 1) degrades (e.g., to form M1).

In some embodiments, a composition described herein is subcutaneously administered to the individual and less than 30% of the compound of Formula I degrades (e.g., subcutaneously). In some embodiments, a composition described herein is subcutaneously administered to the individual by subcutaneous (bolus) injection and less than 30% of the compound of Formula I degrades (e.g., subcutaneously). In some embodiments, a composition described herein is subcutaneously administered to the individual by subcutaneous infusion and less than 30% of the compound of Formula I degrades (e.g., subcutaneously). In some embodiments, the compound of Formula I degrades subcutaneously to form M1.

In some embodiments, the composition is subcutaneously infused into the individual and less M1 is formed relative to administration of an otherwise identical composition administered by subcutaneous (bolus) injection.

In some embodiments, the composition is subcutaneously infused into the individual and less M1 is formed systemically relative to administration of an otherwise identical composition administered by subcutaneous (bolus) injection.

In some embodiments, the composition is subcutaneously infused into the individual and less M1 is formed locally (injection/infusion site) relative to administration of an otherwise identical composition administered by subcutaneous (bolus) injection.

In some embodiments, subcutaneously infusing the composition into the individual improves tolerability. In some embodiments, subcutaneously infusing the composition into the individual improves tolerability relative to subcutaneous (bolus) injection, such as based on a reduction of M1 overproduction, such as subcutaneously.

In some embodiments, subcutaneously infusing the composition into the individual reduces undesired systemic events, such as undesired vasoconstriction that results in ischemia. In some embodiments, subcutaneously infusing the composition into the individual reduces undesired administration site events, such as local site vasoconstriction that result in administration site ischemia. In some embodiments, subcutaneously infusing the composition into the individual reduces undesired systemic events and undesired administration site events.

Provided in some embodiments herein is a method of reducing (incidence of) local vasoconstriction in an individual in need thereof. In some embodiments, the method is a method for reducing the incidence of local vasoconstriction in an individual in need thereof. In some embodiments, the method is a method of reducing (incidence of) ischemia in an individual in need thereof. In some embodiments, the method is a method of reducing (incidence of) injection site ischemia in an individual in need thereof. In some embodiments, the method comprises subcutaneously infusing into the individual in need thereof a composition comprising a compound described herein, such as a compound having a structure represented by Formula I, or a pharmaceutically acceptable salt thereof.

In some instances, a compound described herein (e.g., Compound 1) is used to treat complications of ESLD.

Provided in some embodiments herein is a method of modulating mean arterial pressure (MAP in an individual (e.g., in need thereof), the method comprising subcutaneously infusing into the individual (e.g., in need thereof) a composition comprising an (effective) amount of a compound described herein, such as a mixed vasopressin receptor 1A (V1AR) agonist-antagonist described herein. In some embodiments, the compound has a structure represented by Formula I. In some embodiments, the compound is Compound 1.

In some embodiments, the method further comprises affixing a subcutaneous infusion device to the skin (e.g., skin surface) of the individual. In some embodiments, the subcutaneous infusion device comprises a chamber body and a hollow tube body. In some embodiments, the composition is configured within the chamber body. In some embodiments, the hollow tube body comprises a first opening and a second opening. In some embodiments, the first opening is in fluid contact with the chamber body. In some embodiments, the second opening is configured subcutaneously within the individual after affixing the subcutaneous infusion device to the skin. In some embodiments, the hollow tube is a needle, such as having any gauge suitable for subcutaneous administration (e.g., subcutaneous infusion).

In some embodiments, the subcutaneous infusion device further comprises a pump configured to subcutaneously infuse the composition into the individual at a constant rate. In some embodiments, the subcutaneous infusion device further comprises a pump configured to subcutaneously infuse the composition into the individual at a varying rate. In some embodiments, the rate is a flow rate. In some embodiments, the continuous infusion is at a flow that is insufficient to provide a stream of the composition. In some embodiments, the continuous infusion is at a flow that provides a continuous drip to the individual. In some embodiments, the composition is subcutaneously infused into the individual at a rate of about 0.001 milliliters per hour (mL/hr) or more. In some embodiments, the composition is subcutaneously infused into the individual at a rate of about 1 mL/hr or less. In some embodiments, the composition is subcutaneously infused into the individual at a rate of about 0.005 mL/hr to about 1 mL/hr for an administration period. In some embodiments, the composition is subcutaneously infused into the individual at a rate of about 0.01 mL/hr to about 1 mL/hr for an administration period. In some embodiments, the composition is subcutaneously infused into the individual at a rate of about 0.04 mL/hr to about 1 mL/hr for an administration period. In some embodiments, the administration period is for at least about one hour. In some embodiments, the administration period is for at least about one day. In some embodiments, the administration period is for at least about one week. In some embodiments, the administration period is for at least about one month. In some embodiments, the administration period is about one month or more. In some embodiments, the administration period is about two months or more. In some embodiments, the administration period is about three months or more. In some embodiments, the administration period is about four months or more. In some embodiments, the administration period is about five months or more. In some embodiments, the administration period is about six months or more. In some embodiments, the administration period is about nine months or more. In some embodiments, the administration period is about twelve months or more.

In some embodiments, a composition described herein is subcutaneously infused into the individual for at least one hour. In some embodiments, the composition is subcutaneously infused into the individual for at least one day. In some embodiments, the composition is subcutaneously infused into the individual for at least one week. In some embodiments, the composition is subcutaneously infused into the individual for at least one month.

In some embodiments, a composition described herein is subcutaneously infused into the individual continuously for at least one hour. In some embodiments, the composition is subcutaneously infused into the individual continuously for at least one day. In some embodiments, the composition is subcutaneously infused into the individual continuously for at least one week. In some embodiments, the composition is subcutaneously infused into the individual continuously for at least one month.

In some embodiments, a compound described herein is administered to the individual (e.g., continuously) in an amount of about 0.001 milligram (mg) to about 100 mg. In some embodiments, the compound is administered to the individual (e.g., continuously) in an amount of about 0.01 mg to about 50 mg. In some embodiments, the compound is administered to the individual (e.g., continuously) in an amount of about 0.01 mg to about 20 mg. In some embodiments, the compound is administered to the individual (e.g., continuously) in an amount of about 0.01 mg to about 10 mg. In some embodiments, the compound is administered to the individual (e.g., continuously) in an amount of about 0.1 mg to about 1 mg. In some embodiments, the compound is administered to the individual (e.g., continuously) in an amount of about 0.2 mg. In some embodiments, the composition is administered to the individual over a period of one or more days.

In some embodiments, a composition described herein comprises the compound in a concentration of about 0.001 milligrams per milliliters (mg/mL) or more. In some embodiments, the composition comprises the compound in a concentration of about 100 mg/mL or less. In some embodiments, the composition comprises the compound in a concentration of about 0.001 mg/mL to about 100 mg/mL. In some embodiments, the composition comprises the compound in a concentration of about 0.01 mg/mL to about 100 mg/mL. In some embodiments, the composition comprises the compound in a concentration of about 0.1 mg/mL to about 100 mg/mL. In some embodiments, the composition comprises the compound in a concentration of about 1 mg/mL to about 100 mg/mL. In some embodiments, the composition comprises the compound in a concentration of about 0.1 mg/mL to about 50 mg/mL. In some embodiments, the composition comprises the compound in a concentration of about 1 mg/mL to about 50 mg/mL. In some embodiments, the composition comprises the compound in a concentration of about 0.1 mg/mL to about 10 mg/mL. In some embodiments, the composition comprises the compound in a concentration of about 1 mg/mL to about 10 mg/mL.

In some embodiments, a compound described herein is administered to an individual described in need thereof at a dose of about 0.1 milligram (mg)/day. In some embodiments, a compound described herein is administered to an individual described in need thereof at a dose of about 100 mg/day. In some embodiments, a compound described herein is administered to an individual described in need thereof at a dose of about 0.1 mg/day to about 100 mg/day. In some embodiments, a compound described herein is administered to an individual described in need thereof at a dose of about 0.1 mg/day to about 50 mg/day. In some embodiments, a compound described herein is administered to an individual described in need thereof at a dose of about 1 mg/day to about 50 mg/day. In some embodiments, a compound described herein is administered to an individual described in need thereof at a dose of about 1 mg/day to about 10 mg/day. In some embodiments, the compound is administered to the individual continuously.

Provided in some embodiments herein is a method of modulating mean arterial pressure (MAP) in an individual, the method comprising subcutaneously administering to the individual an effective amount of a compound, the compound being a mixed vasopressin receptor 1A (V1AR) agonist-antagonist. In some embodiments, the individual is in need of an increase in MAP. In some embodiments, the individual has end-stage liver disease (ESLD) or a complication thereof.

In some embodiments, the MAP of the individual increases (e.g., compared to a baseline measurement before treatment) following subcutaneous administration of a compound described herein (e.g., Compound 1). In some embodiments, the MAP of the individual increases (e.g., compared to a baseline measurement before treatment) following subcutaneous administration of Compound 1.

In some embodiments, the MAP of the individual increases by about 1% or more (e.g., compared to a baseline measurement before treatment) after administering the compound, or the pharmaceutically acceptable salt thereof, described herein (e.g., Compound 1). In some embodiments, the MAP of the individual increases by about 5% or more (e.g., compared to a baseline measurement before treatment) after administering the compound, or the pharmaceutically acceptable salt thereof, described herein (e.g., Compound 1). In some embodiments, the MAP of the individual increases by about 10% or more (e.g., compared to a baseline measurement before treatment) after administering the compound, or the pharmaceutically acceptable salt thereof, described herein (e.g., Compound 1). In some embodiments, the MAP of the individual increases by about 15% or more (e.g., compared to a baseline measurement before treatment) after administering the compound, or the pharmaceutically acceptable salt thereof, described herein (e.g., Compound 1). In some embodiments, the MAP of the individual increases by about 20% or more (e.g., compared to a baseline measurement before treatment) after administering the compound, or the pharmaceutically acceptable salt thereof, described herein (e.g., Compound 1). In some embodiments, the MAP of the individual increases by about 1% to about 20% (e.g., compared to a baseline measurement before treatment) after administering the compound, or the pharmaceutically acceptable salt thereof, described herein (e.g., Compound 1). In some embodiments, the MAP of the individual increases by about 5% to about 20% (e.g., compared to a baseline measurement before treatment) after administering the compound, or the pharmaceutically acceptable salt thereof, described herein (e.g., Compound 1). In some embodiments, the MAP of the individual increases by about 10% to about 20% (e.g., compared to a baseline measurement before treatment) after administering the compound, or the pharmaceutically acceptable salt thereof, described herein (e.g., Compound 1). In some embodiments, the MAP of the individual increases by about 1% to about 10% (e.g., compared to a baseline measurement before treatment) after administering the compound, or the pharmaceutically acceptable salt thereof, described herein (e.g., Compound 1). In some embodiments, the increase in MAP occurs about one or more hours after the compound, or the pharmaceutically acceptable salt thereof, is administered to the individual. In some embodiments, the increase in MAP occurs about two or more hours after the compound, or the pharmaceutically acceptable salt thereof, is administered to the individual. In some embodiments, the increase in MAP occurs about three or more hours after the compound, or the pharmaceutically acceptable salt thereof, is administered to the individual. In some embodiments, the increase in MAP occurs about four or more hours after the compound, or the pharmaceutically acceptable salt thereof, is administered to the individual. In some embodiments, the increase in MAP occurs about five or more hours after the compound, or the pharmaceutically acceptable salt thereof, is administered to the individual. In some embodiments, the increase in MAP occurs about six or more hours after the compound, or the pharmaceutically acceptable salt thereof, is administered to the individual. In some embodiments, the increase in MAP occurs about twelve or more hours after the compound, or the pharmaceutically acceptable salt thereof, is administered to the individual. In some embodiments, the increase in MAP occurs about one to twelve hours after the compound, or the pharmaceutically acceptable salt thereof, is administered to the individual. In some embodiments, the increase in MAP occurs about one to six hours after the compound, or the pharmaceutically acceptable salt thereof, is administered to the individual. In some embodiments, the increase in MAP occurs about four to six hours after the compound, or the pharmaceutically acceptable salt thereof, is administered to the individual.

In some embodiments, the MAP of the individual dose-dependently increases after administering the compound, or the pharmaceutically acceptable salt thereof.

In some embodiments, a compound provided herein has a maximal therapeutic concentration. In some embodiments, the maximal therapeutic concentration comprises an increase in concentration that does not provide dose-dependent increases in MAP. In some embodiments, a compound provided herein has a non-linear dose dependency, such as above the maximal therapeutic concentration. In some embodiments, a dose dependent increase in MAP includes the maximal therapeutic concentration. In some embodiments, dose dependently includes situations where the effect increases with dose for at least a certain dose range. For example, higher doses may provide no or lower dose-dependent increases.

In some embodiments, a compound described herein (e.g., Compound 1) is a selective vasopressin V1a receptor partial agonist. In some embodiments, a compound described herein (e.g., Compound 1) is a mixed agonist-antagonist. In some embodiments, a compound described herein (e.g., Compound 1) is a selective for V1AR. In some embodiments, a compound described herein (e.g., Compound 1) is a selective for V1AR over vasopressin 2 (V2) receptor (V2R). In some instances, a compound described herein (e.g., Compound 1) has no functional V2R activity. In some instances, a compound described herein (e.g., Compound 1) has no functional V2R activity at therapeutic concentrations. In some embodiments, the therapeutic concentration is a concentration sufficient to modulate V1AR.

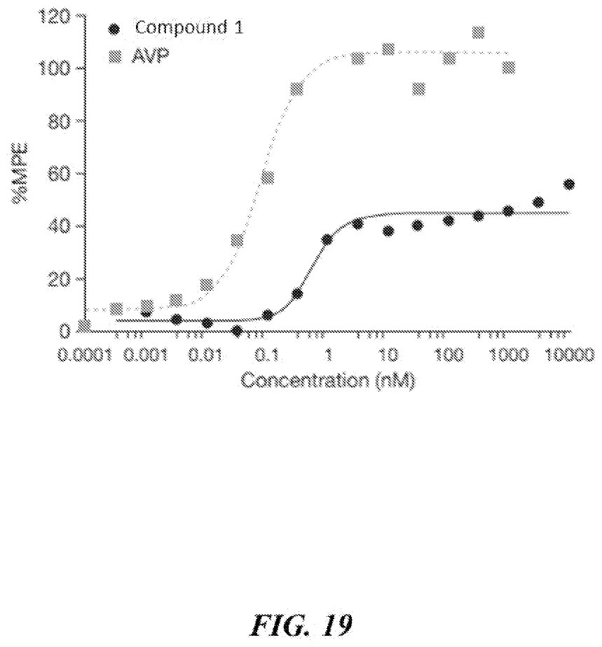

In some instances, the activity and selectivity of a compound described herein (e.g., a mixed agonist-antagonist, such as Compound 1) is demonstrated by Tables 14 and 15. In some instances, the activity and selectivity of a compound described herein (e.g., a mixed agonist-antagonist, such as Compound 1) is demonstrated by FIGS. 19-21.

In some embodiments, a compound described herein (e.g., Compound 1) is selective for V1AR over V2R by more than 10-fold. In some embodiments, a compound described herein (e.g., Compound 1) is selective for V1AR over V2R by more than 100-fold. In some embodiments, a compound described herein (e.g., Compound 1) is selective for V1AR over V2R by more than 1,000-fold. In some embodiments, a compound described herein (e.g., Compound 1) is selective for V1AR over V2R by more than 10,000-fold. In some embodiments, a compound described herein (e.g., Compound 1) is inactive at V2R.

In some embodiments, a compound described herein (e.g., Compound 1) comprises a first portion having agonist activity. In some embodiments, a compound described herein (e.g., Compound 1) comprises a second portion having antagonist activity. In some embodiments, a compound described herein (e.g., Compound 1) comprises a first portion having agonist activity and a second portion having antagonist activity.

In some embodiments, a compound provided herein has a ratio of agonist to antagonist activity of about 90:10 to about 10:90. In some embodiments, a compound provided herein has a ratio of agonist to antagonist activity of about 50:50.

In some embodiments, an agonist-antagonists refers to a compound having an agonist portion and an antagonist portion. In specific embodiments, the agonist portion and the antagonist portion are discrete.

In some embodiments, a V1AR agonist-antagonist has a wider therapeutic window than a V1AR agonist. In some embodiments, a V1AR agonist-antagonist described herein has a selective V1a agonist portion and a selective V1a antagonist portion. In some instances, either the selective V1a agonist portion or the selective V1a antagonist portion binds to V1AR, such that both the selective V1a agonist portion and the selective V1a antagonist portion do not bind to the V1AR simultaneously. In some instances, the V1a antagonist portion competes with the selective V1a agonist portion for binding to V1AR. In some instances, V1AR agonism provides a (desired) vasoconstrictive effect. In some instances, V1AR antagonism prevents maximal activation of the V1a pathway.

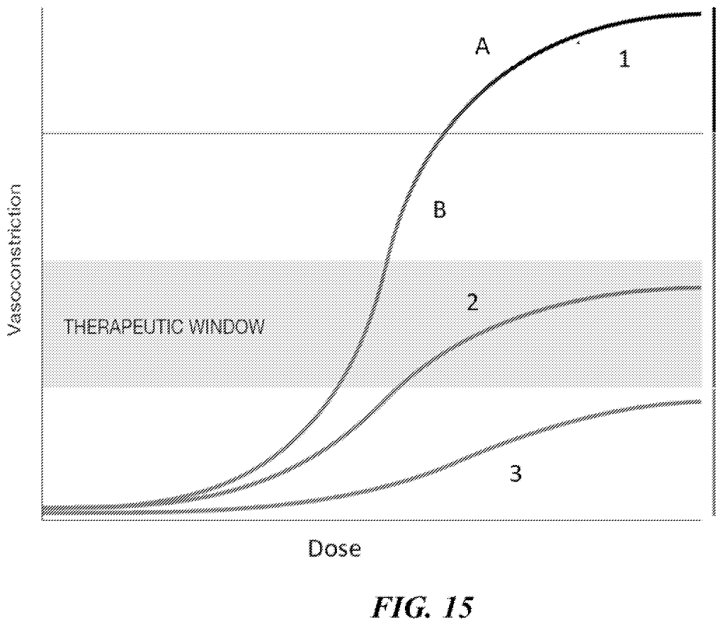

In some instances, FIG. 15, curve 1 illustrates a concentration-response curve for a compound (e.g., a full, nonselective (V2, V1a) agonist, such as terlipressin) that provides a lethal level of vasoconstriction (e.g., at relatively high doses, depicted as portion A in FIG. 15) and/or serious adverse events (e.g., at doses above therapeutic levels and at doses below lethal levels of vasoconstriction, depicted as portion B in FIG. 15). In some embodiments, a compound described herein (e.g., a full, nonselective (V2, V1a) agonist, such as terlipressin) has a concentration-response curve illustrated in FIG. 15, line 1. In some instances, FIG. 15, line 1 illustrates that a compound described herein (e.g., a full, nonselective (V2, V1a) agonist, such as terlipressin) has a relatively narrow therapeutic window. In some instances, FIG. 15, line 1 illustrates that at relatively high doses, a compound described herein (e.g., a full, nonselective (V2, V1a) agonist, such as terlipressin) provides a level of vasoconstriction that is lethal (depicted as portion A in FIG. 15) and/or associated with serious adverse events (depicted as portion B in FIG. 15), such as elevated lactate and/or vasoconstriction with ischemia.

In some embodiments, FIG. 15, curve 2 illustrates a concentration-response curve for a compound (e.g., a mixed V1a agonist-antagonist, such as Compound 1) that has a safe and efficacious profile. In some embodiments, a compound described herein (e.g., a mixed V1a agonist-antagonist, such as Compound 1) has a concentration-response curve illustrated in FIG. 15, line 2. In some instances, FIG. 15, line 2 illustrates that even at high doses, a compound described herein (e.g., a mixed V1a agonist-antagonist, such as Compound 1) has a relatively large therapeutic window. In some instances, FIG. 15, line 2 illustrates that even at high doses, a compound described herein (e.g., a mixed V1a agonist-antagonist, such as Compound 1) is safe and efficacious.

In some instances, FIG. 15, curve 3 illustrates a concentration-response curve for a compound that does not reach therapeutic levels. In some instances, FIG. 15, curve 3 illustrates a concentration-response curve for a full, nonselective (V2, V1a) agonist or a partial V1a agonist, such as a compound that has relatively low activity for V1AR.

In some instances, FIGS. 15-21 demonstrate that a compound described herein (e.g., a mixed V1a agonist-antagonist, such as Compound 1) can be safely used (over a large dose range) to treat ESLD or symptoms and/or complications thereof. In some instances, FIG. 15 demonstrates that even at excessively high concentrations, a compound described herein (e.g., a mixed V1a agonist-antagonist, such as Compound 1) provides a maximal effect (e.g., a change (increase) in MAP) in an individual (e.g., after subcutaneous administration). In some instances, FIG. 15-21 demonstrate that a compound described herein (e.g., a mixed V1a agonist-antagonist, such as Compound 1) provides a robust effect (e.g., change (increase) in MAP) in an individual (e.g., after subcutaneous administration). In some instances, FIGS. 15-21 demonstrate that a compound described herein (e.g., a mixed V1a agonist-antagonist, such as Compound 1) reaches, maintains, and does not go above a safe and efficacious therapeutic effect (e.g., change (increase) in MAP), such as after subcutaneous administration. In some instances, FIGS. 15-21 demonstrate that a compound described herein (e.g., a mixed V1a agonist-antagonist, such as Compound 1) maintains (a safe level of) therapeutic efficacy (e.g., after subcutaneous administration) over a prolonged period of time, such as for at least 10 minutes, 20 minutes, 30 minutes, 40 minutes, 50 minutes, 60 minutes, 70 minutes, 80 minutes, 90 minutes, or 100 minutes or more. In some instances, FIGS. 15-21 demonstrate that a compound described herein (e.g., a full, nonselective (V2, V1a) agonist, such as terlipressin) quickly reaches toxic and potentially harmful concentrations, such as at relatively high doses. In some instances, FIGS. 15-21 demonstrate that a compound described herein (e.g., a full, nonselective (V2, V1a) agonist, such as terlipressin) the effect of full, nonselective (V2, V1a) agonists diminishes rapidly, such as quickly falling below therapeutic levels after a relatively short period of time (e.g., after about 80 mins or more).

In some embodiments, a compound described herein (e.g., Compound 1) modulates (e.g., increases) mean arterial pressure (MAP). In some embodiments, a compound described herein (e.g., Compound 1) is administered subcutaneously and modulates (e.g., increases) MAP. In some embodiments, a compound described herein (e.g., Compound 1) is administered subcutaneously and modulates (e.g., increases) MAP without (significant) injection site reactions (e.g., local vasoconstriction), such as at a subcutaneous injection site. In some embodiments, a compound described herein (e.g., Compound 1) is suitable for subcutaneous administration for the treatment of one or more complication(s) of ESLD, such as HRS-AKI.

In some embodiments, the modulation of mean arterial pressure (MAP) in the individual comprises raising MAP by at least 5% over baseline (e.g., such as a level prior to administration of a compound described herein (e.g., Compound 1)). In some embodiments, the modulation of MAP in the individual comprises raising MAP by at least 10% over baseline (e.g., such as a level prior to administration of a compound described herein (e.g., Compound 1)). In some embodiments, the modulation of MAP in the individual comprises raising MAP by at least 15% over baseline (e.g., such as a level prior to administration of a compound described herein (e.g., Compound 1)). In some embodiments, baseline is a level prior to administration of a compound described herein (e.g., Compound 1). In some instances, baseline is a level compared to a control, such as a placebo.

In some embodiments, the modulation of mean arterial pressure (MAP) in the individual comprises raising MAP by at most 5% over baseline (e.g., such as a level prior to administration of a compound described herein (e.g., Compound 1)). In some embodiments, the modulation of MAP in the individual comprises raising MAP by at most 10% over baseline (e.g., such as a level prior to administration of a compound described herein (e.g., Compound 1)). In some embodiments, the modulation of MAP in the individual comprises raising MAP by at most 15% over baseline (e.g., such as a level prior to administration of a compound described herein (e.g., Compound 1)). In some embodiments, baseline is a level prior to administration of a compound described herein (e.g., Compound 1). In some instances, baseline is a level compared to a control, such as a placebo.

In some embodiments, the modulation of MAP in the individual comprises raising MAP by at least 5 mmHg over baseline (e.g., such as a level prior to administration of a compound described herein (e.g., Compound 1)). In some embodiments, the modulation of MAP in the individual comprises raising MAP by 1 mmHg or more over baseline (e.g., such as a level prior to administration of a compound described herein (e.g., Compound 1)). In some embodiments, the modulation of MAP in the individual comprises raising MAP by 5 mmHg or more over baseline (e.g., such as a level prior to administration of a compound described herein (e.g., Compound 1)). In some embodiments, the modulation of MAP in the individual comprises raising MAP by 10 mmHg or more over baseline (e.g., such as a level prior to administration of a compound described herein (e.g., Compound 1)). In some embodiments, the modulation of MAP in the individual comprises raising MAP by about 20 mmHg or more over baseline (e.g., such as a level prior to administration of a compound described herein (e.g., Compound 1)). In some embodiments, baseline is a level prior to administration of a compound described herein (e.g., Compound 1).

Provided in some embodiments herein is a method of modulating mean arterial pressure (MAP) in an individual (e.g., as described herein above), the method comprising subcutaneously administering to the individual an effective amount of a compound having a structure represented by Formula I: D1-L-D2. In some embodiments, D1 is a vasopressin receptor 1A (V1AR) agonist. In some embodiments, D2 is a V1AR antagonist. In some embodiments, L is a linker. In some embodiments, the compound is administered to the individual as a pharmaceutically acceptable salt.

In some embodiments, a compound described herein (e.g., Compound 1) has a structure represented by Formula I: D1-L-D2. In some embodiments, D1 is a vasopressin receptor 1A (V1AR) agonist. In some embodiments, D2 is a V1AR antagonist. In some embodiments, L is a linker. In some embodiments, the compound is administered to the individual as a pharmaceutically acceptable salt.

In some embodiments, D1 is selective for V1AR. In some embodiments, D1 is selective for V1AR over V2R. In some embodiments, D1 is selective for V1AR over V2R by more than 10-fold. In some embodiments, D1 is selective for V1AR over V2R by more than 100-fold. In some embodiments, D1 is selective for V1AR over V2R by more than 1,000-fold. In some embodiments, D1 is selective for V1AR over V2R by more than 10,000-fold. In some embodiments, D1 is inactive at V2R.

In some embodiments, D1 comprises a peptide. In some embodiments, D1 is a peptide. In some embodiments, D1 comprises a cyclic peptide. In some embodiments, D1 is a cyclic peptide. In some embodiments, D1 comprises a cyclic nonapeptide. In some embodiments, D1 is a cyclic nonapeptide.

In some embodiments, D1 has or comprises the following structure:

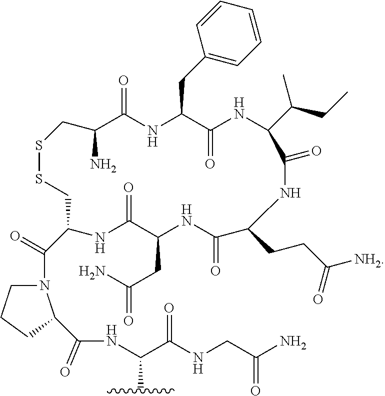 [SMILES: *[C@H](NC(=O)[C@@H]1CCCN1C(=O)[C@@H]1CSSC[C@H](N)C(=O)N[C@@H](Cc2ccccc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

In some embodiments, D1 has or comprises the following structure:

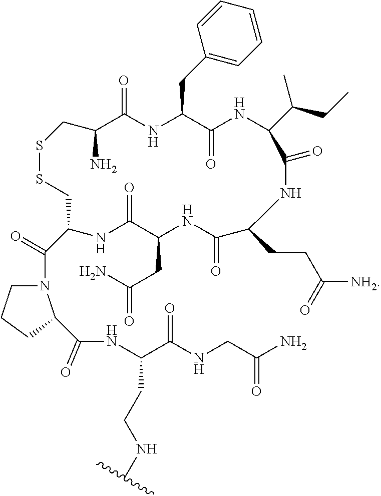 [SMILES: *NCC[C@H](NC(=O)[C@@H]1CCCN1C(=O)[C@@H]1CSSC[C@H](N)C(=O)N[C@@H](Cc2ccccc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

In some instances, D2 is selective for V1AR. In some embodiments, D2 is selective for V1AR over V2R. In some instances, D2 is selective for V1AR over V2R by more than 10-fold. In some instances, D2 is selective for V1AR over V2R by more than 100-fold. In some instances, D2 is selective for V1AR over V2R by more than 1,000-fold. In some instances, D2 is selective for V1AR over V2R by more than 10,000-fold. In some instances, D2 is inactive at V2R.

In some embodiments, at least one of D1 and D2 is selective for V1AR. In some embodiments, at least one of D1 and D2 is selective for V1AR over V2R.

In some embodiments, D2 comprises a peptide. In some embodiments, D2 is a peptide. In some embodiments, D2 comprises a linear peptide. In some embodiments, D2 is a linear peptide. In some embodiments, is a linear polypeptide comprising about seven or more amino acid residues. In some embodiments, D2 comprises seven to twelve amino acid residues.

In some embodiments, D2 has or comprises the following structure:

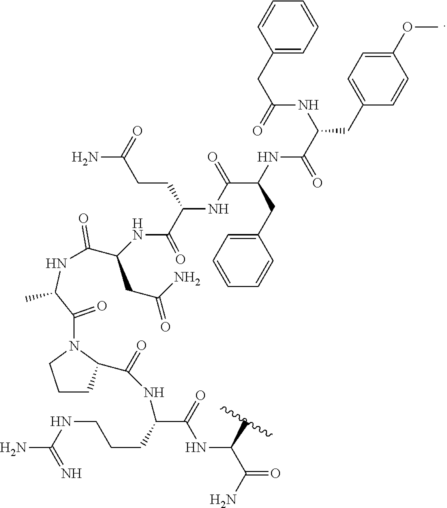 [SMILES: *[C@H](NC(=O)[C@H](CCCNC(=N)N)NC(=O)[C@@H]1CCCN1C(=O)[C@H](C)NC(=O)[C@H](CC(N)=O)NC(=O)[C@H](CCC(N)=O)NC(=O)[C@H](Cc1ccccc1)NC(=O)[C@@H](Cc1ccc(OC)cc1)NC(=O)Cc1ccccc1)C(N)=O]

In some embodiments, D2 has or comprises the following structure:

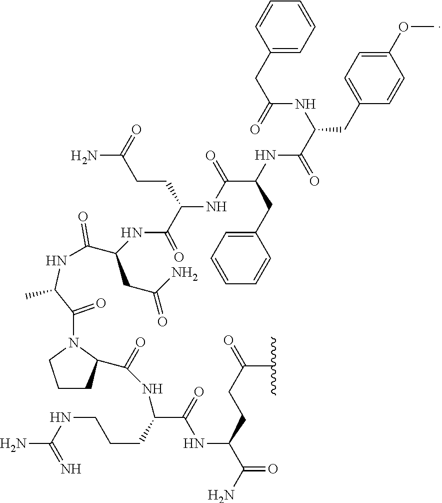 [SMILES: *C(=O)CC[C@H](NC(=O)[C@H](CCCNC(=N)N)NC(=O)[C@H]1CCCN1C(=O)[C@H](C)NC(=O)[C@H](CC(N)=O)NC(=O)[C@H](CCC(N)=O)NC(=O)[C@H](Cc1ccccc1)NC(=O)[C@@H](Cc1ccc(OC)cc1)NC(=O)Cc1ccccc1)C(N)=O]

In some embodiments, L is a non-hydrolyzable linker.

In some embodiments, L comprises a peptide bond. In some embodiments, L comprises one or more amino acid residue. In some embodiments, L is one or more amino acid residue. In some embodiments, L comprises one or more modified amino acid residue. In some embodiments, L is one or more modified amino acid residue.

In some embodiments, L comprises one or more linker group, each linker group being independently selected from the group consisting of a substituted or unsubstituted alkyl and substituted or unsubstituted heteroalkyl. In some embodiments, L is a bond, substituted or unsubstituted alkyl, or substituted or unsubstituted heteroalkyl. In some embodiments, L is or comprises substituted or unsubstituted heteroalkyl. In some embodiments, L is heteroalkyl substituted with one or more substituent, each substituent being independently selected form the group consisting of oxo, amino, and substituted heteroalkyl. In some embodiments, L is alkylamine substituted with one or more substituent, each substituent being independently selected form the group consisting of oxo, amino, and substituted heteroalkyl. In some embodiments, L is alkylamine substituted with oxo.

In some embodiments, L has or comprises the following structure:

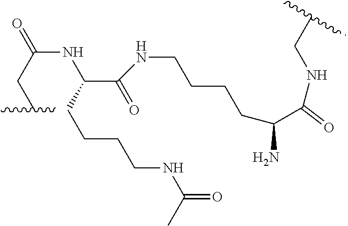 [SMILES: *CNC(=O)[C@@H](N)CCCCNC(=O)[C@H](CCCCNC(C)=O)NC(=O)C*]

In some embodiments, L has or comprises the following structure:

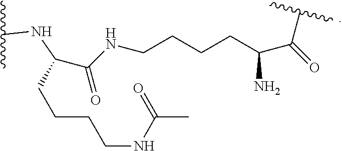 [SMILES: *N[C@@H](CCCCNC(C)=O)C(=O)NCCCC[C@H](N)C(*)=O]

In some embodiments, the compound described herein is Compound 1, or a pharmaceutically acceptable salt thereof.

In some instances, Compound 1 is Glycinamide, L-cysteinyl-L-phenylalanyl-L-isoleucyl-L-glutaminyl-L-asparaginyl-L-cysteinyl-L-prolyl-N4-(phenylacetyl-O-methyl-D-tyrosyl-L-phenylalanyl-L-glutaminyl-L-asparaginyl-L-alanyl-L-prolyl-L-arginyl-L-isoglutamyl-N5-acetyl-L-lysyl-L-ε-lysyl)-L-2,4-diaminobutyryl-, cyclic (1→6)-disulfide.

In some instances, Compound 1 has an empirical molecular formula of C₁₁₀H₁₆₁N₃₁O₂₇S₂.

In some instances, Compound 1 has an average molecular mass of 2413.78 u.

In some instances, a mixed V1A agonist-antagonist provided herein (e.g., Compound 1) is a white to off-white powder.

In some instances, a mixed V1A agonist-antagonist provided herein (e.g., Compound 1) has a solubility in water of at least 10 mg/mL.

In some embodiments, Compound 1 has a structure represented by Formula (I-A):

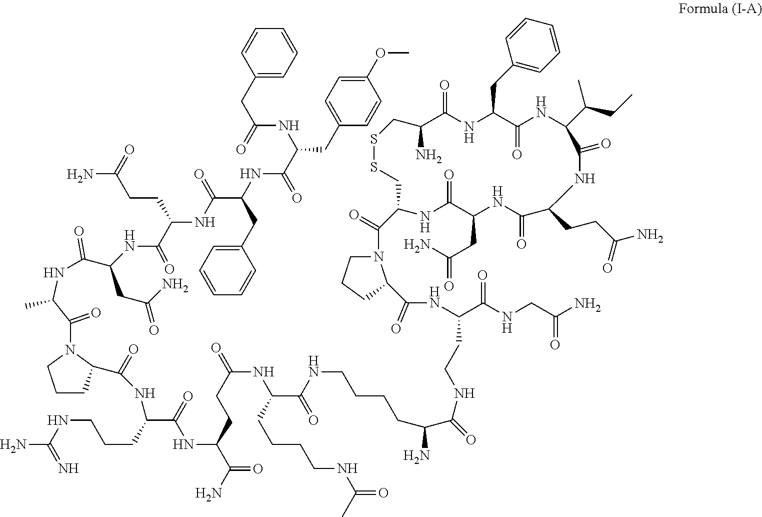 [SMILES: CC[C@H](C)[C@@H]1NC(=O)[C@H](Cc2ccccc2)NC(=O)[C@@H](N)CSSC[C@@H](C(=O)N2CCC[C@H]2C(=O)N[C@@H](CCNC(=O)[C@@H](N)CCCCNC(=O)[C@H](CCCCNC(C)=O)NC(=O)CC[C@H](NC(=O)[C@H](CCCNC(=N)N)NC(=O)[C@@H]2CCCN2C(=O)[C@H](C)NC(=O)[C@H](CC(N)=O)NC(=O)[C@H](CCC(N)=O)NC(=O)[C@H](Cc2ccccc2)NC(=O)[C@@H](Cc2ccc(OC)cc2)NC(=O)Cc2ccccc2)C(N)=O)C(=O)NCC(N)=O)NC(=O)[C@H](CC(N)=O)NC(=O)[C@H](CCC(N)=O)NC1=O]

or a pharmaceutically acceptable salt thereof.

In some instances, a mixed V1A agonist-antagonist provided herein (e.g., Compound 1) is a 20-mer monocyclic, branched peptide, such as, containing natural and unnatural amino acids, such as, from non-animal origin. In some instances, a mixed V1A agonist-antagonist provided herein (e.g., Compound 1) has an S—S bridge between the Cys¹ and Cys⁶ residues. In some instances, the branch is linked through the position 8 side chain amino function.

In some instances, provided herein is a compound having a structure represented by Formula (I-B):

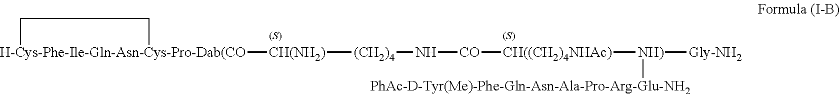

or a pharmaceutically acceptable salt thereof,

wherein:
Dab is 2,4-diamino butyric acid, D-Tyr(Me) is O-methyl-D-tyrosine and
PhAc is phenylacetic acid (e.g., wherein L-2,4-diamino butyric acid, N-ε-acetyl-L-lysine, L-isoglutamine and O-methyl-D-tyrosine are unnatural, and the N-terminal moiety is substituted with phenylacetic acid).

In some instances, a mixed V1A agonist-antagonist provided herein (e.g., Compound 1) is provided as a pharmaceutically acceptable salt. In some instances, a mixed V1A agonist-antagonist provided herein (e.g., Compound 1) is provided as an acetate salt. In some instances, a mixed V1A agonist-antagonist provided herein (e.g., Compound 1) is administered in the form described in Example 1.

In some embodiments, a compound described herein is any compound described in any of U.S. Pat. No. 9,644,000 or U.S. Pat. No. 9,388,214, each of which is incorporated herein by reference, in their entirety, in particular for the compounds provided therein.

In some instances, Compound 1 has an empirical molecular formula of C₁₁₀H₁₆₁N₃₁O₂₇S₂(AcOH)z, where z is any integer (e.g., from 1-100).

Provided in some embodiments herein is a method of modulating mean arterial pressure (MAP) in an individual (e.g., as described herein), the method comprising subcutaneously administering to the individual an effective amount of Compound 1, or a pharmaceutically acceptable salt thereof.

Provided in some embodiments herein is a pharmaceutical composition comprising an amount of a compound, or a pharmaceutically acceptable salt thereof, described herein (e.g., Compound 1). In some embodiments, the amount of the compound is an effective amount of the compound. In some embodiments, the compound is a mixed vasopressin receptor 1A (V1AR) agonist-antagonist. In some embodiments, the composition is formulated for subcutaneous administration. In some embodiments, the compound has a structure represented by Formula I. In some embodiments, the compound is Compound 1.

Provided in some embodiments herein is a subcutaneous formulation comprising a compound described herein, such as a compound having a structure represented by Formula I, or a pharmaceutically acceptable salt thereof.

In some embodiments, less than 50% of the compound, such as the compound of Formula I, degrades. In some embodiments, less than 50% of the compound, such as the compound of Formula I, degrades to form M1. In some embodiments, less than 50% of the compound, such as the compound of Formula I, degrades subcutaneously. In some embodiments, less than 50% of the compound, such as the compound of Formula I, degrades to form M1 subcutaneously.

In some embodiments, less than 30% of the compound, such as the compound of Formula I, degrades. In some embodiments, less than 30% of the compound, such as the compound of Formula I, degrades to form M1. In some embodiments, less than 30% of the compound, such as the compound of Formula I, degrades subcutaneously. In some embodiments, less than 30% of the compound, such as the compound of Formula I, degrades to form M1 subcutaneously.

In some embodiments, a composition described herein further comprises a liquid vehicle or solvent (e.g., water or an aqueous vehicle).

Provided in some embodiments herein is a subcutaneous formulation having a concentration of a compound described herein, such a compound having a structure represented by Formula I, of about 0.001 milligrams (mg)/milliliters (mL) or more. In some embodiments, the subcutaneous formulation has a concentration of a compound described herein, such a compound having a structure represented by Formula I, of about 100 mg/mL or less. In some embodiments, the subcutaneous formulation has a concentration of a compound described herein, such a compound having a structure represented by Formula I, of about 0.001 mg/mL 100 mg/mL. In some embodiments, the subcutaneous formulation comprises the compound in a concentration of about 0.01 mg/mL to about 100 mg/mL. In some embodiments, the subcutaneous formulation comprises the compound in a concentration of about 0.1 mg/mL to about 100 mg/mL. In some embodiments, the subcutaneous formulation comprises the compound in a concentration of about 1 mg/mL to about 100 mg/mL. In some embodiments, the subcutaneous formulation comprises the compound in a concentration of about 0.1 mg/mL to about 50 mg/mL. In some embodiments, the subcutaneous formulation comprises the compound in a concentration of about 1 mg/mL to about 50 mg/mL. In some embodiments, the subcutaneous formulation comprises the compound in a concentration of about 0.1 mg/mL to about 10 mg/mL. In some embodiments, the subcutaneous formulation comprises the compound in a concentration of about 1 mg/mL to about 10 mg/mL.

In some embodiments, the compound is formulated as described in the Examples herein. In some embodiments, the compound is formulated as an aqueous solution. In some embodiments, the compound is formulated as an aqueous solution having a concentration of the compound of at least about 0.01 mg/mL. In some embodiments, the compound is formulated as an aqueous solution having a concentration of the compound of at least about 0.1 mg/mL. In some embodiments, the compound is formulated as an aqueous solution having a concentration of the compound of at least about 1 mg/mL. In some embodiments, the compound is formulated as an aqueous solution having a concentration of the compound of at least about 10 mg/mL. In some embodiments, the compound is formulated as an aqueous solution having a concentration of the compound of at most about 50 mg/mL. In some embodiments, the compound is formulated as an aqueous solution having a concentration of the compound of at most about 10 mg/mL. In some embodiments, the compound is formulated as an aqueous solution having a concentration of the compound of at most about 1 mg/mL. In some embodiments, the compound is formulated as an aqueous solution having a concentration of the compound of at most about 0.1 mg/mL. In some embodiments, the compound is formulated as an aqueous solution having a concentration of the compound of at most about 0.01 mg/mL. In some embodiments, the compound is formulated as an aqueous solution having a concentration of the compound of about 0.01 mg/mL to about 50 mg/mL. In some embodiments, the compound is formulated as an aqueous solution having a concentration of the compound of about 0.01 mg/mL to about 10 mg/mL. In some embodiments, the compound is formulated as an aqueous solution having a concentration of the compound of about 1 mg/mL to about 10 mg/mL. In some embodiments, the composition is formulated with acetate buffer.

In some embodiments, the composition is formulated at a pH of 4.5. In some embodiments, the composition is formulated with mannitol.

In some embodiments, additives (e.g., preservatives) were added to a (e.g., subcutaneous) composition having a pH of about 3-6 (e.g., 4.5) and comprising at least about 1 millimolar (mM) acetate buffer (e.g., about 5 mM sodium acetate to about 150 mM sodium acetate) and about 10 milligram per milliliters (mg/mL) to about 100 mg/mL mannitol (e.g., about 43.6 mg/mL mannitol). In some instances, the additive is a preservative. In some instances, a preservative is added to a (e.g., subcutaneous) composition having a pH of about 4.5 and comprising about 10 mM sodium acetate and about 43.6 mg/mL mannitol.

In some embodiments, provided herein is a composition comprising Compound 1. In some embodiments, the composition is suitable for subcutaneous administration. In some embodiments, the formulation is suitable for subcutaneous (bolus) injection. In some embodiments, the subcutaneous (bolus) injection is provided to the individual as a single dose (e.g., all at once). In some embodiments, the formulation is suitable for subcutaneous infusion.

In some embodiments, a composition described herein comprises an additive. In some embodiments, a composition described herein is a subcutaneous composition and comprises an additive. In some embodiments, the additive is selected from the group consisting of a preservative, a solubilizing agent (e.g., a cyclodextrin), a buffering agent, and a chelating agent (e.g., zinc acetate or ethylenediaminetetraacetic acid (EDTA)). In some embodiments, the additive is a preservative.

Provided in some embodiments herein is a subcutaneous formulation comprising a compound having a structure represented by Formula I and a preservative.

In some embodiments, a composition described herein further comprises a preservative. In some embodiments, the preservative is any suitable preservative, such as meta (m)-cresol, phenol, chlorobutanol, or benzyl alcohol. In some embodiments, the preservative is present in the composition in an amount of about 0.1 mg/mL or more. In some embodiments, the preservative is present in the composition in an amount of about 50 mg/mL or less. In some embodiments, the preservative is present in the composition in an amount of about 0.1 mg/mL to about 50 mg/mL. In some embodiments, the preservative is present in the composition in an amount of about 1 mg/mL to about 20 mg/mL.

In some instances, a composition comprising a preservative described herein showed no physical interaction (e.g., aggregation) with any screened preservatives (e.g., between the preservative and Compound 1), even at relatively high concentrations of the preservative.

Provided in some embodiments herein is a subcutaneous formulation comprising a compound having a structure represented by Formula I and a solubilizing agent.

In some embodiments, a composition described herein further comprises a solubilizing agent. In some embodiments, the preservative is any suitable solubilizing agent, such as a cyclodextrin (e.g., sulfobutylether-β-cyclodextrin (SBECD)). In some embodiments, the solubilizing agent is present in the composition in an amount of about 0.1 mg/mL or more. In some embodiments, the solubilizing agent is present in the composition in an amount of about 500 mg/mL or less. In some embodiments, the solubilizing agent is present in the composition in an amount of about 250 mg/mL or less. In some embodiments, the solubilizing agent is present in the composition in an amount of about 100 mg/mL or less. In some embodiments, the solubilizing agent is present in the composition in an amount of about 0.1 mg/mL to about 250 mg/mL. In some embodiments, the solubilizing agent is present in the composition in an amount of about 0.1 mg/mL to about 100 mg/mL. In some embodiments, the solubilizing agent is present in the composition in an amount of about 1 mg/mL to about 250 mg/mL. In some embodiments, the solubilizing agent is present in the composition in an amount of about 1 mg/mL to about 100 mg/mL. In some embodiments, the solubilizing agent is present in the composition in an amount of about 60 mg/mL to about 80 mg/mL.

In some embodiments, the composition further comprises a buffering agent. In some embodiments, the buffering agent has a pKa of about 3.0 to about 6.0, such as at 25° C. In some embodiments, the buffering agent is selected from the group consisting of acetate buffer, succinate buffer, phosphate buffer, and citrate buffer. In some embodiments, the composition further comprises acetate buffer. In some embodiments, the acetate buffer is a combination of acetate and acetic acid. In some embodiments, the composition further comprises succinate buffer. In some embodiments, the succinate buffer is a combination of succinate and succinic acid. In some embodiments, the composition further comprises citrate buffer. In some embodiments, the citrate buffer is a combination of citrate and citric acid.

Provided in some embodiments herein is a subcutaneous formulation comprising a compound having a structure represented by Formula I and a buffering agent at a concentration of about 1 millimolar (mM) to about 1 M.

In some embodiments, a composition described herein comprises a buffering agent in a concentration of about 1 millimolar (mM) or more. In some embodiments, a composition described herein comprises a buffering agent in a concentration of about 1 molar (M) or less. In some embodiments, a composition described herein comprises a buffering agent in a concentration of about 1 mM to about 1 M. In some embodiments, a composition described herein comprises a buffering agent in a concentration of about 1 mM to about 500 mM. In some embodiments, a composition described herein comprises a buffering agent in a concentration of about 1 mM to about 250 mM. In some embodiments, a composition described herein comprises a buffering agent in a concentration of about 50 mM to about 250 mM. In some embodiments, a composition described herein comprises a buffering agent in a concentration of about 50 mM to about 150 mM. In some embodiments, a composition described herein comprises a buffering agent in a concentration of about 100 mM. In some embodiments, a composition described herein comprises a buffering agent in a concentration of about 1 mM to about 50 mM. In some embodiments, a composition described herein comprises a buffering agent in a concentration of about 5 mM to about 25 mM. In some embodiments, a composition described herein comprises a buffering agent in a concentration of about 10 mM.

In some embodiments, a composition described herein has a pH sufficient to inhibit (e.g., deactivate or inactivate) a protease (e.g., trypsin), such as in subcutaneous layer of an individual subcutaneously administered the formulation.

In some embodiments, the pH of a composition described herein does not (substantially) change when administered subcutaneously to an individual. In some embodiments, the pH of a composition described herein does not (substantially) change when administered subcutaneously to an individual by subcutaneous (bolus) injection. In some embodiments, the pH of a composition described herein does not (substantially) change when administered subcutaneously to an individual subcutaneous infusion).

In some embodiments, a composition described herein has a pH of at least about 3. In some embodiments, a composition described herein has a pH of about 9 or less, 8 or less, 7 or less, 6 or less, 5 or less, or 4 or less. In some embodiments, a composition described herein has a pH of about 4 to about 8. In some embodiments, a composition described herein has a pH of about 4 to about 6. In some embodiments, a composition described herein has a pH of about 4.5 to about 5. In some embodiments, a composition described herein has a pH of about 4. In some embodiments, a composition described herein has a pH of about 4.5.

In some embodiments, a composition described herein has an ionic strength of about 1 mM or more. In some embodiments, a composition described herein has an ionic strength of about 1 M or less. In some embodiments, a composition described herein has an ionic strength of about 1 mM to about 500 mM. In some embodiments, a composition described herein has an ionic strength of about 5 mM to about 200 mM. In some embodiments, a composition described herein has an ionic strength of about 5 mM to about 10 mM to about 100 mM.

In some embodiments, a compound described herein, such as a compound having a structure represented by Formula I, is (e.g., substantially) less susceptible to (e.g., protease) degradation. In some embodiments, a compound described herein, such as a compound having a structure represented by Formula I, is (e.g., substantially) less susceptible to (e.g., protease) degradation in the subcutaneous layer of an individual subcutaneously administered a composition comprising the compound.

In some embodiments, a compound described herein, such as a compound having a structure represented by Formula I, is stable in a composition described herein. In some embodiments, less than 50% of a compound described herein, such as a compound having a structure represented by Formula I, degrades in a vial. In some embodiments, less than 50% of a compound described herein, such as a compound having a structure represented by Formula I, degrades in a vial over a period of at least about one week (e.g., about one week or more, two week or more, three weeks or more, or four weeks or more). In some embodiments, less than 50% of a compound described herein, such as a compound having a structure represented by Formula I, degrades subcutaneously. In some embodiments, less than 50% of a compound described herein, such as a compound having a structure represented by Formula I, degrades subcutaneously over a period of at least about one hour (e.g., about one hour or more, six hours or more, twelve hours or more, or twenty-four hours or more).

In some embodiments, a composition described herein, such as having an acidic pH, a relatively high buffer (e.g., acetate buffer) concentration, and/or a relatively high concentration of a compound described herein (e.g., Compound 1) is well-tolerated after subcutaneous (e.g., injection or infusion) administration (see Examples 3 and 4).

In some embodiments, a composition described herein comprises a pH and/or buffer (e.g., acetate buffer) concentration that is sufficient to inhibit, reduce, or eliminate formation of undesirable metabolites of a compound described herein (e.g., Compound 1), such as full (V1a) agonists like M1. In some instances, compositions comprising relatively high buffer concentrations (e.g., 50 mM or more of the buffer) prolong the amount of time a local environment (e.g., at or near the injection site) maintains a certain pH (e.g., a pH of about 3 or more) that is sufficient to inhibit, reduce or eliminate formation of undesirable metabolites of a compound described herein (e.g., Compound 1), such as full (V1a) agonists like M1. In some instances, compositions having an acidic pH (e.g., pH of 1 to 6) produce undesirable effects when (subcutaneously) injected, such effects including injection site burning, stinging, pain, or the like. Injecting a composition (subcutaneously) that has a relatively acidic pH or a buffer concentration that prolongs the amount of time a local environment (e.g., at or near the injection site) maintains a certain (e.g., acidic) pH would be expected to prolong such undesirable effects. In contrast, compositions described herein that have a relatively acidic pH (e.g., a pH of 4 or 4.5) and/or a buffer concentration that prolongs the amount of time a local environment (e.g., at or near the injection site) maintains a certain (e.g., acidic) pH do not produce (noticeable) undesirable effects when administered subcutaneously (e.g., by injection or infusion). In some instances, higher buffer concentrations inhibit, reduce, or eliminate formation of M1 to provide sufficient amount of time for absorption of a compound described herein, such as due to an extended time that the pH of the local environment is acidic (e.g., 4.5) after administration. In some embodiments, the buffer concentration of a composition provided herein that prolongs the amount of time a local environment (e.g., at or near the injection site) maintains a certain (e.g., acidic) is about 50 mM or more. In some embodiments, the pH of a composition provided herein that sufficiently inhibits, reduces, or eliminates formation of undesirable metabolites of a compound described herein (e.g., Compound 1), such as full (V1a) agonists like M1, when administered subcutaneously (e.g., by injection or infusion) is acidic. In some embodiments, the pH of a composition provided herein that sufficiently inhibits, reduces, or eliminates formation of undesirable metabolites of a compound described herein (e.g., Compound 1), such as full (V1a) agonists like M1, when administered subcutaneously (e.g., by injection or infusion) is about 3 or more. In some embodiments, the pH of a composition provided herein that sufficiently inhibits, reduces, or eliminates formation of undesirable metabolites of a compound described herein (e.g., Compound 1), such as full (V1a) agonists like M1, when administered subcutaneously (e.g., by injection or infusion) is about 4 or 4.5.

In some embodiments, a composition described herein (e.g., a composition suitable for intravenous or subcutaneous administration) comprises acetate buffer in a concentration of about 10 mM or more. In some embodiments, a composition described herein comprises acetate buffer in a concentration of about 50 mM or more.

In some embodiments, a composition described herein (e.g., a composition suitable for intravenous or subcutaneous administration) has a pH of about 3 or more.

In some embodiments, a composition described herein (e.g., a composition suitable for intravenous or subcutaneous administration) comprises acetate buffer in a concentration of 10 mM and has a pH of 4.5. In some embodiments, the composition is suitable for intravenous administration. In some embodiments, the composition does not comprise a preservative. In some embodiments, the composition is suitable for subcutaneous administration. In some embodiments, the composition is suitable for subcutaneous infusion.

In some embodiments, a composition described herein (e.g., a composition suitable for subcutaneous administration) comprises acetate buffer in a concentration of 100 mM (or more) and has a pH of about 4 or 4.5. In some embodiments, the composition is suitable for subcutaneous administration. In some embodiments, the composition is suitable for subcutaneous (bolus) injection. In some embodiments, the composition is suitable for subcutaneous infusion.

In some embodiments, a composition described herein (e.g., a composition suitable for intravenous or subcutaneous administration) comprises acetate buffer in a concentration of about 10 mM or more. In some embodiments, a composition described herein comprises acetate buffer in a concentration of about 50 mM or more.

In some embodiments, a composition described herein comprises a concentration of a compound described herein (e.g., Compound 1) that is sufficient to inhibit, reduce, or eliminate formation of undesirable metabolites of the compound, such as full (V1a) agonists like M1. In some instances, compositions comprising relatively high drug concentrations (e.g., 0.1 milligrams/milliliter (mg/mL) or more) saturate a local environment (e.g., at or near the injection site) with drug such that more of the drug absorbs before substantial formation of undesirable metabolites of the drug, such as full (V1a) agonists. In some embodiments, compositions described herein that have a relatively high concentration of a compound provided herein sufficiently inhibits, reduces, or eliminates formation of undesirable metabolites of a compound described herein (e.g., Compound 1), such as full (V1a) agonists like M1, when administered subcutaneously (e.g., by injection or infusion). In some embodiments, the drug concentration (e.g., the concentration of a compound described herein) in the composition is about 0.1 mg/mL or more.

In some embodiments, a composition provided herein has a concentration of a compound described herein that is about 0.1 mg/mL or more. In some embodiments, a composition provided herein has a concentration of a compound described herein that is about 1 mg/mL or more. In some embodiments, a composition provided herein has a concentration of a compound described herein that is about 100 mg/mL or less. In some embodiments, a composition provided herein has a concentration of a compound described herein that is about 0.1 mg/mL to about 100 mg/mL. In some embodiments, a composition provided herein has a concentration of a compound described herein that is about 0.1 mg/mL to about 10 mg/mL. In some embodiments, the composition is suitable for subcutaneous administration. In some embodiments, the composition is suitable for subcutaneous (bolus) injection. In some embodiments, the composition is suitable for subcutaneous infusion. In some embodiments, a composition provided herein has a concentration of a compound described herein that is about 0.1 mg/mL or less. In some embodiments, the composition is suitable for intravenous administration.

In some embodiments, a composition described herein is suitable for subcutaneous infusion. In some embodiments, a composition suitable for subcutaneous infusion is administered to an individual in a way that reduces (injection site) injury or trauma. In some embodiments, a composition suitable for subcutaneous infusion is administered to an individual over a certain (prolonged) period of time. In some embodiments, a composition suitable for subcutaneous infusion is administered to an individual at a certain (relatively slow) rate, such as a drip. In some embodiments, the composition is administered at a rate that is sufficient to provide a drip (e.g., not a stream) of the composition. In some embodiments, the composition is administered at a rate that is about 0.1 milliliters per hour (mL/hr) or less, such as over a prolonged period of time (e.g., a period of about 24 hours or more).

Provided in some embodiments herein is a pharmaceutical composition comprising an amount of a compound having a structure represented by Formula I. In some embodiments, the composition is formulated for subcutaneous administration. In some embodiments, the amount of the compound is an effective amount of the compound.

Provided in some embodiments herein is a pharmaceutical composition comprising an amount of Compound 1, or a pharmaceutically acceptable salt thereof, the composition being formulated for subcutaneous administration. In some embodiments, the amount of the compound is an effective amount of the compound.

In some embodiments, a composition described herein is suitable for systemic delivery of an active agent described herein, such as a compound having a structure represented by Formula I. In some embodiments, a composition described herein is suitable for administration of an active agent described herein, such as a compound having a structure represented by Formula I, in an out-patient setting, such as at-home. In some embodiments, a composition described herein is suitable for systemic delivery of Compound 1. In some embodiments, a composition described herein is suitable for administration of Compound 1 in an out-patient setting, such as at-home.

Provided in some embodiments herein is a system for modulating mean arterial pressure, the system comprising a composition comprising a compound of Formula I, or a pharmaceutically acceptable salt thereof and a device configured to provide subcutaneous infusion of the composition to an individual when the device is positioned on the skin of the individual.

In some embodiments, the system comprises an adhesive body for affixing the (subcutaneous infusion) device to the surface of the skin of the individual. In some embodiments, the system comprises an adhesive body for reversibly affixing the (subcutaneous infusion) device to the surface of the skin of the individual.

In some embodiments, the system comprises a chamber body and a hollow tube body. In some embodiments, the composition is configured within the chamber body. In some embodiments, wherein the hollow tube body comprises a first opening and a second opening. In some embodiments, the first opening is in fluid contact with the chamber body. In some embodiments, the second opening is configured subcutaneously within the individual after affixing the subcutaneous infusion device to the skin of the individual. In some embodiments, the hollow tube is a needle, such as having any gauge suitable for subcutaneous administration (e.g., subcutaneous infusion).

In some embodiments, the system does not comprise an adhesive body. In some embodiments, the device is not attached to the surface of the skin of the individual. In some embodiments, the chamber body is attached to an injection port. In some embodiments, the system does not comprise an adhesive body and the chamber body is attached to an injection port.

In some embodiments, the (subcutaneous infusion) device further comprises a pump configured to subcutaneously infuse the composition into the individual at a constant or varying rate (e.g., as described herein). In some embodiments, the system is configured to provide the composition to the individual over a period of about 24 hours or more. In some embodiments, the system is configured to continuously provide the composition to the individual over a period of about 24 hours or more.

In some embodiments, the device is configured to receive a vial and/or a cartridge of the composition.

In some embodiments, the device is a subcutaneous infusion device. In some embodiments, the device is a pump.

In some instances, provided herein is a pharmaceutical composition comprising an effective amount of a first compound, or a pharmaceutically acceptable salt thereof, and an effective amount of a second compound, or a pharmaceutically acceptable salt thereof, wherein the first compound is a vasoconstrictor, and wherein the second compound sufficiently blocks a (e.g., local) vasoconstrictive effect of the first compound, such as providing sufficient uptake of the first compound to the circulatory system and/or an internal organ (e.g., a kidney) of an individual (e.g., in need thereof). In some embodiments, the pharmaceutical composition is injectable. In some instances, the pharmaceutical composition is suitable for intravitreal administration. In some instances, the pharmaceutical composition is suitable for subcutaneous administration. In some instances, the first compound is a vasopressin receptor 1A (V1AR) agonist. In some instances, the first compound is a selective V1AR agonist. In some instances, the second compound is a V1AR antagonist. In some instances, the second compound is a selective V1AR antagonist. In some instances, the second compound is a vasodilator.

In some embodiments, a compound described herein (e.g., Compound 1) is safe (e.g., in humans) (e.g., see Example 1). In some embodiments, a compound described herein (e.g., Compound 1) is well tolerated (e.g., in humans) (e.g., see Example 1). In some embodiments, a compound described herein (e.g., Compound 1) is safe and well tolerated (e.g., in humans) (e.g., see Example 1). In some embodiments, a compound described herein (e.g., Compound 1) has a pharmacodynamic profile that has a submaximal partial agonism consistent with a mixed agonist-antagonist of the V1a receptor.

In some instances, a compound described herein (e.g., Compound 1) is tested in a phase 1, double-blinded, placebo-controlled, within dose-group randomized trial to investigate the safety, tolerability, and pharmacokinetic and pharmacodynamic (PD) profiles of the compound administered to healthy adults aged 18 to 45 years (see Example 1). In some instances, the trial comprised 2 treatment periods: Period 1 (6-h intravenous [IV] infusion of the compound over a dose range of 0.1 to 0.9 mg or placebo) and Period 2 (once-daily subcutaneous [SC] injection of the compound 0.1, 0.3 mg, or placebo for 5 consecutive days). In some instances, Period 1 included 32 men and women. In some instances, 8 men and 5 women continued into Period 2. In some instances, such as after IV administration, exposure, as measured by AUC and Cₘₐₓ was roughly dose proportional over the dose range studied. In some instances, such as after SC administration, Tₘₐₓ was 0.3 h, exposure was more than proportional, and bioavailability was 18% without any apparent accumulation after repeated administration. In some instances, the terminal half-life (t_{1/2}) of a compound described herein (e.g., Compound 1) was about 1.5 h and 1.0 h after IV and SC administration, respectively, such as indicating that absorption is not rate-limiting for elimination after SC administration. In some instances, diastolic and, to a lesser extent, systolic blood pressure (BP) increased for subjects treated with a compound described herein (e.g., Compound 1) in all dose groups, while pulse rate decreased. In some instances, overall changes in mean arterial pressure (MAP) after IV and SC administration were similar to changes in diastolic BP. In some instances, absolute changes in cardiac output, by echocardiography, appeared to be dose dependent, with mean reductions of 3%-12% after 0.9 mg IV dose, and individual reductions ≤20-25% across any dose. In some instances, adverse events (AEs) included abdominal pain and diarrhea, with laboratory tests negative for and no reported cases of mesenteric ischemia. In some instances, AEs were related to treatment, generally mild or moderate in severity and attributable to the expected pharmacologic effect.

In some embodiments, diastolic blood pressure of an individual increases (e.g., compared to a baseline measurement before treatment) after administration of a compound, or the pharmaceutically acceptable salt thereof, described herein (e.g., Compound 1). In some embodiments, diastolic blood pressure of the individual increases (e.g., compared to a baseline measurement before treatment) after subcutaneous administration of a compound, or the pharmaceutically acceptable salt thereof, described herein (e.g., Compound 1).

In some embodiments, systolic blood pressure of an individual increases (e.g., compared to a baseline measurement before treatment) after administration of a compound, or the pharmaceutically acceptable salt thereof, described herein (e.g., Compound 1). In some embodiments, systolic blood pressure of the individual increases (e.g., compared to a baseline measurement before treatment) after subcutaneous administration of a compound, or the pharmaceutically acceptable salt thereof, described herein (e.g., Compound 1).

In some embodiments, diastolic and/or systolic blood pressure of an individual dose-dependently increases (e.g., compared to a baseline measurement before treatment) after administration of a compound, or the pharmaceutically acceptable salt thereof, described herein (e.g., Compound 1). In some embodiments, diastolic and/or systolic blood pressure of the individual dose dependently increases (e.g., compared to a baseline measurement before treatment) after subcutaneous administration of a compound, or the pharmaceutically acceptable salt thereof, described herein (e.g., Compound 1).

In some embodiments, a compound provided herein has a maximal therapeutic concentration. In some embodiments, the maximal therapeutic concentration comprises an increase in concentration that does not provide dose-dependent increases in blood pressure, such as diastolic blood pressure, systolic blood procedure, and/or MAP. In some embodiments, a compound provided herein has a non-linear dose dependency, such as above the maximal therapeutic concentration. In some embodiments, a dose dependent increase in blood pressure, such as diastolic blood pressure, systolic blood procedure, and/or MAP, includes the maximal therapeutic concentration. In some embodiments, dose dependently includes situations where the effect increases with dose for at least a certain dose range. For example, higher doses may provide no or lower dose-dependent increases.

In some embodiments, MAP is calculated from measurements of systolic blood pressure and diastolic blood pressure. In some embodiments, MAP is calculated when taking into account the pressure during a single cardiac cycle. In some embodiments, MAP is ⅓ (systolic blood pressure−diastolic blood pressure)+diastolic blood pressure.

In some embodiments, the pulse rate of the individual decreases (e.g., compared to a baseline measurement before treatment) after administration of a compound, or the pharmaceutically acceptable salt thereof, described herein (e.g., Compound 1). In some embodiments, the peripheral blood flow of the individual decreases (e.g., compared to a baseline measurement before treatment) after administration of a compound, or the pharmaceutically acceptable salt thereof, described herein (e.g., Compound 1). In some embodiments, the pulse rate and peripheral blood flow of the individual decreases (e.g., compared to a baseline measurement before treatment) after administration of a compound, or the pharmaceutically acceptable salt thereof, described herein (e.g., Compound 1).

In some embodiments, administration of a compound, or the pharmaceutically acceptable salt thereof, described herein (e.g., Compound 1) to an individual (e.g., described herein) improves systemic hemodynamics in the individual. In some embodiments, subcutaneous administration of a compound, or the pharmaceutically acceptable salt thereof, described herein (e.g., Compound 1) to an individual (e.g., described herein) improves systemic hemodynamics in the individual.

In some embodiments, administration of a compound, or the pharmaceutically acceptable salt thereof, described herein (e.g., Compound 1) to an individual (e.g., described herein) reduces fluid retention in the individual. In some embodiments, administration of a compound, or the pharmaceutically acceptable salt thereof, described herein (e.g., Compound 1) to an individual (e.g., described herein) reduces fluid overload in the individual. In some embodiments, administration of a compound, or the pharmaceutically acceptable salt thereof, described herein (e.g., Compound 1) to an individual (e.g., described herein) reduces fluid retention and overload in the individual. In some embodiments, subcutaneous administration of a compound, or the pharmaceutically acceptable salt thereof, described herein (e.g., Compound 1) to an individual (e.g., described herein) reduces fluid retention in the individual. In some embodiments, subcutaneous administration of a compound, or the pharmaceutically acceptable salt thereof, described herein (e.g., Compound 1) to an individual (e.g., described herein) reduces fluid overload in the individual. In some embodiments, subcutaneous administration of a compound, or the pharmaceutically acceptable salt thereof, described herein (e.g., Compound 1) to an individual (e.g., described herein) reduces fluid retention and overload in the individual.

In some embodiments, (e.g., subcutaneous) administration of a compound, or the pharmaceutically acceptable salt thereof, described herein (e.g., Compound 1) to an individual (e.g., described herein) reduces serum creatinine (sCr) (value) in the individual (e.g., compared to a baseline measurement before treatment). In some embodiments, a compound, or the pharmaceutically acceptable salt thereof, described herein (e.g., Compound 1) is (e.g., subcutaneously) administered to an individual (e.g., described herein) at least until the individual has a sCr value of 1.5 milligrams (mg)/deciliters (dL) or less. In some embodiments, a compound, or the pharmaceutically acceptable salt thereof, described herein (e.g., Compound 1) is (e.g., subcutaneously) administered to an individual (e.g., described herein) at least until the sCr value of the individual returns to normal (e.g., baseline).

In some instance, intravenous (i.v.) administration of a compound described herein (e.g., Compound 1) provides a terminal half-life of about 1.5 hours with clearance and distribution volumes of about 13 L/h and about 15-20 L, respectively. In some instances, the tₘₐₓ after subcutaneous (s.c.) administration of a compound described herein (e.g., Compound 1) is about 0.3 hours with a terminal half-life of about 1 hour (e.g., without any accumulation after repeated administrations). In some instances, the bioavailability after subcutaneous (s.c.) administration of a compound described herein (e.g., Compound 1) is about 18%.

In some instances, a compound described herein (e.g., Compound 1) has a proportional increase in exposure, by means of AUC and Cₘₐₓ, following i.v. administration. In some instances, a compound described herein (e.g., Compound 1) has more than approximate proportionality following s.c. administration. In some instances, a compound described herein (e.g., Compound 1) has a higher exposure in women than in men, such as after both i.v. infusion and subcutaneous injection.

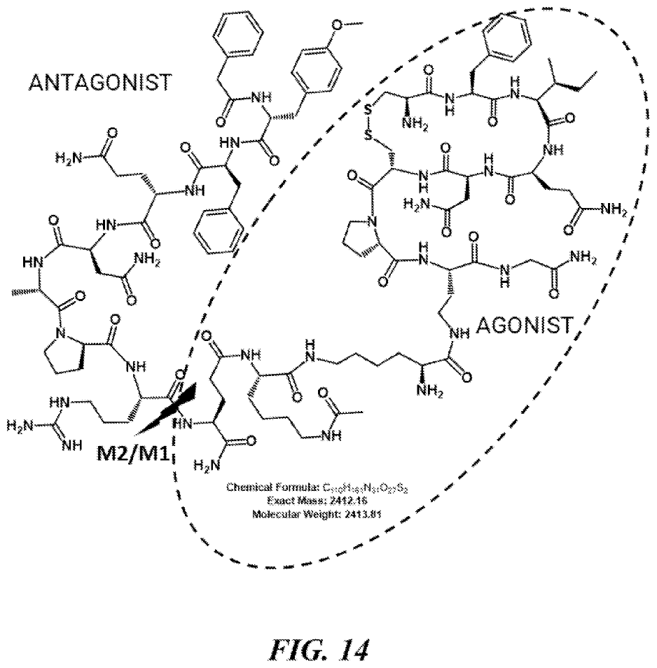

In some instances, a compound described herein (e.g., Compound 1) metabolizes to metabolite M1 (see FIG. 14). M1 has a structure that is the free form of the structure circled in FIG. 14. In some embodiments, M1 is a full (VIA) agonist. In some embodiments, M1 has a molecular weight of 1405.68 g/mol. In some embodiments, M1 has a chemical formula of C₆₀H₉₈N₁₉O₁₆S₂⁺. M1 is (substantially) less active (for V1a) than Compound 1. Specifically, M1 has about one-tenth of the activity for V1a as Compound 1.

In some instances, such as in single subjects after i.v. administration, M1 is found in the plasma of an individual administered Compound 1. In some instances, such as in all investigated subjects at comparable concentrations to Compound 1 after s.c. administration, M1 is found. In some instances, a compound described herein (e.g., Compound 1) is metabolized (e.g., to M1) during the transport to the circulation after s.c. injection. As discussed hereinabove, full agonists described herein are known to be toxic and lead to (serious) adverse events when administered subcutaneously. As such, formation of a full agonist (e.g., M1), such as when a compound described herein is administered by subcutaneous (bolus) injection, is undesirable, such as when being used for the purposes described herein.

In some instances, a dose-independent increase in diastolic, and to a lesser extent, systolic blood pressure is more pronounced after s.c. injection compared with i.v. infusion of a compound described herein (e.g., Compound 1) (e.g., accompanied by a reflex decrease in pulse rate). In some instances, the peripheral blood flow decreases after intravenous and subcutaneous administration of a compound described herein (e.g., Compound 1).

In some instances, an individual receiving a dose of a compound described herein (e.g., Compound 1) had an adverse event (AE) (e.g., related to a treatment). In some instances, an AE occurs at the beginning of a s.c. treatment period. In some instances, there are more AEs after s.c. administration compared with i.v. administration. In some instances, there are more AEs in women compared with men. In some instances, the AEs are of mild or moderate intensity. In some instances, the AEs are of severe intensity. In some instances, an AE of severe intensity is elevated troponin I of mild intensity.

In some instances, a compound described herein (e.g., Compound 1) induces a reversible increase in diastolic. In some instances, a compound described herein (e.g., Compound 1) induces a reversible increase in systolic. In some instances, a compound described herein (e.g., Compound 1) induces a reversible increase in MAP. In some instances, a compound described herein (e.g., Compound 1) induces a reversible increase in diastolic and MAP. In some instances, a compound described herein (e.g., Compound 1) induced a decrease in heart rate and cardiac output. In some instance, evaluation of ECG, clinical chemistry, hematology, hemostasis, and urinalysis parameters did not provide any safety concerns of a compound described herein (e.g., Compound 1).

In some instance, such as when administered as a single i.v. infusion, a compound described herein (e.g., Compound 1) is safe and well tolerated (e.g., in both men and women), such as at a dose of about 0.9 mg or less. In some instances, the maximum tolerated s.c. dose (MTD) is about 0.1 mg of a compound described herein (e.g., Compound 1).

In some instances, the pharmacokinetic parameters for a compound described herein (e.g., Compound 1) (e.g., in plasma for the 0.1 to 0.9 mg i.v. dose groups) provides dose proportionality. In some instances, the pharmacokinetic parameters for a compound described herein (e.g., Compound 1) (e.g., in plasma for the 0.1 to 0.9 mg i.v. dose groups) provides that the dose independent PK parameters are comparable between dose groups (see Example 1). In some instances, men clear a compound described herein (e.g., Compound 1) at a higher rate than women. In some instances, men administered a compound described herein (e.g., Compound 1) have a lower Cₘₐₓ of the compound than women. In some instances, men administered a compound described herein (e.g., Compound 1) have a lower AUC of the compound than women. In some instances, men administered a compound described herein (e.g., Compound 1) have a lower Cₘₐₓ and AUC of the compound than women.

In some instances, such as after s.c. administration of a compound described herein (e.g., Compound 1), the tₘₐₓ is consistent between the first and the fifth dose. In some instances, such as after s.c. administration of a compound described herein (e.g., Compound 1), the t_{1/2} is consistent between the first and the fifth dose. In some instances, such as after s.c. administration of a compound described herein (e.g., Compound 1), the tₘₐₓ and t_{1/2} are consistent between the first and the fifth dose. In some instances, such as after s.c. administration of a compound described herein (e.g., Compound 1), the AUC is variable between the first and the fifth dose. In some instances, such as after s.c. administration of a compound described herein (e.g., Compound 1), the Cₘₐₓ is variable between the first and the fifth dose. In some instances, such as after s.c. administration of a compound described herein (e.g., Compound 1), the AUC and Cₘₐₓ are variable between the first and the fifth dose. In some instance, the dose proportionality for AUC and Cₘₐₓ is proportional (e.g., for Cₘₐₓ at the fifth administration only).

In some instances, the terminal half-life of a compound described herein (e.g., Compound 1) is about 1.5 hours after intravenous administration. In some instances, the terminal half-life of a compound described herein (e.g., Compound 1) is about 1 hour after subcutaneous administration. In some instances, the terminal half-life of a compound described herein (e.g., Compound 1) is comparable in men and women (e.g., suggesting that absorption is not rate limiting for the elimination after s.c. administration, such as also being supported by the short time to Cₘₐₓ after subcutaneous administration, about 0.3 hours, similar in both men and women). In some instances, the bioavailability of a compound described herein (e.g., Compound 1) is about 18%. In some instances, the formation of metabolites after s.c. administration, such as resulting in comparable concentrations of metabolites and the compound, provides a bioavailability of about 18%. In some instances, the fraction of the unchanged, excreted dose in the urine is about <10% after i.v. administration. In some instances, the fraction of the unchanged, excreted dose of a compound described herein (e.g., Compound 1) in the urine is about <5% after s.c. administration. In some instances, the fraction of the unchanged, excreted dose of a compound described herein (e.g., Compound 1) in the urine is constant throughout the dose range (e.g., of the study provided in Example 1).

The presence of the metabolite M1 (a full agonist) in plasma after subcutaneous administration (but only at very low concentrations (<1% of Compound 1) after intravenous administration) demonstrates that Compound 1 is metabolized somewhere on the path between the subcutaneous tissue and the systemic circulation. In some instances, the half-life of M1 is longer than for Compound 1 after i.v. and s.c. administration. In some instances, the half-life of Compound 1 is shorter after s.c. compared with i.v. administration (e.g., about 2 hours and 4 hours, respectively). In some instances, a compound described herein (e.g., Compound 1) metabolizes in the kidneys.

In some instances, the pharmacodynamic effects of a compound described herein (e.g., Compound 1) on blood pressure and heart rate is as expected of a vasopressin V1a specific agonist. In some instances, administration of a compound described herein (e.g., Compound 1) to an individual provides an increase in the diastolic blood pressure of the individual. In some instances, administration of a compound described herein (e.g., Compound 1) to an individual provides an increase in the systolic blood pressure of the individual. In some instances, administration of a compound described herein (e.g., Compound 1) to an individual provides an increase in the MAP of the individual. In some instances, administration of a compound described herein (e.g., Compound 1) to an individual provides a decrease in the pulse of the individual. In some instances, administration of a compound described herein (e.g., Compound 1) to an individual provides a decrease in the peripheral blood flow of the individual. In some instances, the effects on blood pressure and heart rate are dose independent (e.g., the changes that are seen in the lowest dose group were about the same as seen in the higher dose groups). In some instances, s.c. injections provided more pronounced PD effects compared with the same doses given i.v., (e.g., despite an about 50% lower Cₘₐₓ following s.c. administration). In some instances, the active metabolite M1 (e.g., a full V1a agonist) in plasma after s.c. administration contributes (significantly) to the total (e.g., local and/or systemic) V1a vasopressor activity, such as in individuals that have a (serious) adverse event after a compound described herein is administered by subcutaneous (bolus) injection. In some instances, increased M1 formation, such as after subcutaneous (bolus) injection, provides substantially decreases (systemic) delivery of Compound 1, such as resulting in undesirable effects (e.g., due to less delivery of active agent (e.g., M1 or a mixed V1a agonist-antagonist described herein) and/or increased amounts of vasoconstriction, which can increase risk of developing ischemia, cyanosis, pain, inflammation, and/or necrosis).

In some instances, the safety evaluation of a compound described herein (e.g., Compound 1) provided a profile that is improved compared to other (e.g., nonselective) a vasopressin receptor agonists described herein. In some instances, the absolute changes in mean cardiac output are dose dependent. In some instances, the relative changes in mean cardiac output are comparable between doses (e.g., and well separated from the placebo). In some instances, individual decreases in cardiac output are about 20-25% (e.g., as observed in all active treatment groups, including the placebo group, after both i.v. and s.c. administrations). In some instances, the decrease in cardiac output is secondary to a decreased heart rate. In some instances, an ECG evaluation did not indicate any influence of a compound described herein (e.g., Compound 1). In some instances, clinical laboratory parameters did not show any signs of heart or mesenteric ischemia (e.g., or negative effects on the liver or kidney).

In some instances, adverse events in individuals (e.g., intravenously or subcutaneously) receiving a partial agonist described herein (e.g., Compound 1) were comparable to the pharmacological vasopressor activity of compounds described herein (e.g., Compound 1 or vasopressin). For example, subcutaneous administration of a compound described herein (e.g., Compound 1) provided (substantially) more AEs than intravenous administration (see Example 1). Moreover, the frequency of AEs after subcutaneous administration of a compound described herein (e.g., Compound 1) decreases over 5 days of treatment. For example, in some instances, s.c. administration provided about 0.5-fold more AEs per administration on Day 1 than the i.v. administration (e.g., although only 3 subjects received 0.3 mg s.c. compared to 35 subjects receiving 0.45 mg or higher doses i.v.). Additionally, s.c. administration of a partial agonist described herein (e.g., Compound 1) provided about 3-fold more AEs per administration (see Example 1) thank i.v. administration of the same compound. Given that (1) M1 (a full (V1a) agonist) forms after subcutaneous administration of Compound 1, (2) full vasopressin agonists are known to be toxic and lead to adverse events, (3) Compound 1 and M1 are present at about equimolar concentrations after subcutaneous administration of M1, and (4) the exposure to Compound 1 (by means of Cₘₐₓ and AUC) after s.c. administration was lower than after i.v. infusion, the presence of M1 (not Compound 1) in plasma after s.c. administration contributes to the total V1a vasopressor activity and the (e.g., exaggerated) pharmacological effects and/or AE profile after s.c. administration of Compound 1.

Moreover, given that the presence of a vasopressin receptor full agonist, M1, contributed to an increased number of vasoconstriction related AEs compared with much higher concentrations of Compound 1, further demonstrates (in combination with the over a wide concentration range similar pharmacodynamic and adverse effects of Compound 1) that Compound 1 is a partial agonist and/or mixed agonist-antagonist. Further, observations from the study in Example 1 also demonstrate that a mixed agonist-antagonist mechanism of action caps the (local vasoconstrictive) effect(s) in the individual (and limit such effects to a level below the maximally possible). As further support of this explanation, studies with terlipressin, a V1A receptor full agonist, have demonstrated blood pressure increases greater than those provided in Example 1 (e.g., further supporting that the maximal pharmacodynamic effects of Compound 1 are ‘capped’ and that Compound 1 is a V1A receptor partial agonist and/or mixed agonist-antagonist).

In some instances, the pharmacological effects of a compound described herein (e.g., Compound 1) are attributed to vasopressin V1a-receptor agonism. In some instances, the absence of any effect on diuresis and hemostasis demonstrated a high degree of specificity (e.g., providing the desired pharmacological profile) to the V1a-receptor.

In some instances, the terminal half-life of a compound described herein (e.g., Compound 1) is about 1.5 hours (after i.v. administration).

In some instances, the total clearance of a compound described herein (e.g., Compound 1) is about 13 L/h (after i.v. administration).

In some instances, the terminal half-life of a compound described herein (e.g., Compound 1) is about 1 hour (after s.c. administration). In some instances, the tₘₐₓ of a compound described herein (e.g., Compound 1) is about 0.3 hours (after s.c. administration). In some instances, the bioavailability of a compound described herein (e.g., Compound 1) is about 18% (without any apparent accumulation of a compound described herein (e.g., Compound 1), such as after repeated subcutaneous administration).

In some instances, the increase in exposure of a compound described herein (e.g., Compound 1) (e.g., by means of AUC and Cₘₐₓ) is approximately proportional for AUC and Cₘₐₓ after intravenous administration of the compound described herein (e.g., Compound 1).

In some instance, the exposure of a compound described herein (e.g., Compound 1) (by means of AUC and Cₘₐₓ) is higher in women than in men after intravenous infusion of the compound described herein (e.g., Compound 1).

In some instances, administration of a compound described herein (e.g., Compound 1) provided an increase in the diastolic blood pressure of the individual receiving the compound. In some instances, administration of a compound described herein (e.g., Compound 1) provided an increase in the systolic blood pressure of the individual receiving the compound. In some instances, administration of a compound described herein (e.g., Compound 1) provided an increase in the MAP of the individual receiving the compound. In some instances, administration of a compound described herein (e.g., Compound 1) provided a reflex decrease in the pulse rate (e.g., in an apparent dose independent manner) of the individual receiving the compound.

In some instances, the peripheral blood flow decreased after intravenous and subcutaneous administration of a compound described herein (e.g., Compound 1).

In some instances, such as during subcutaneous administration of a compound described herein (e.g., Compound 1), the active metabolite (a full (V1a) agonist) M1 is generated, such as at comparable concentrations to Compound 1.

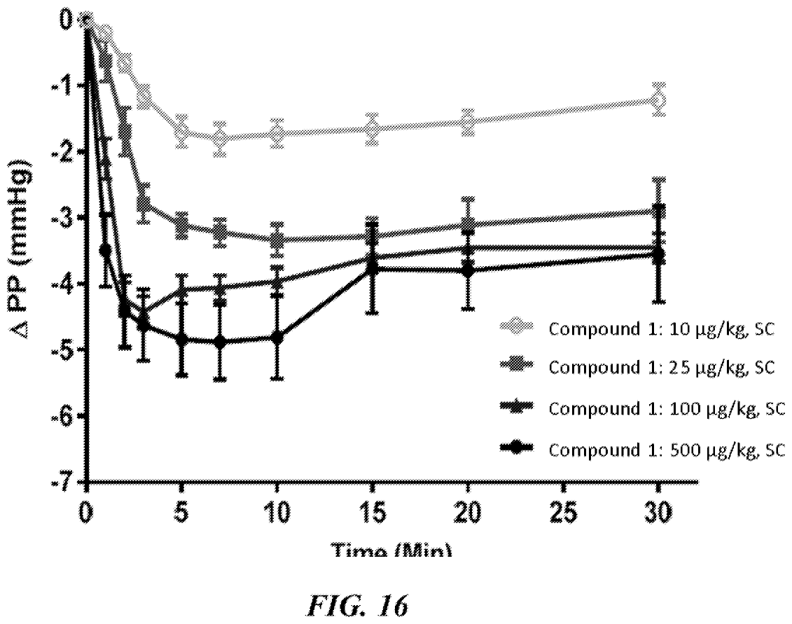

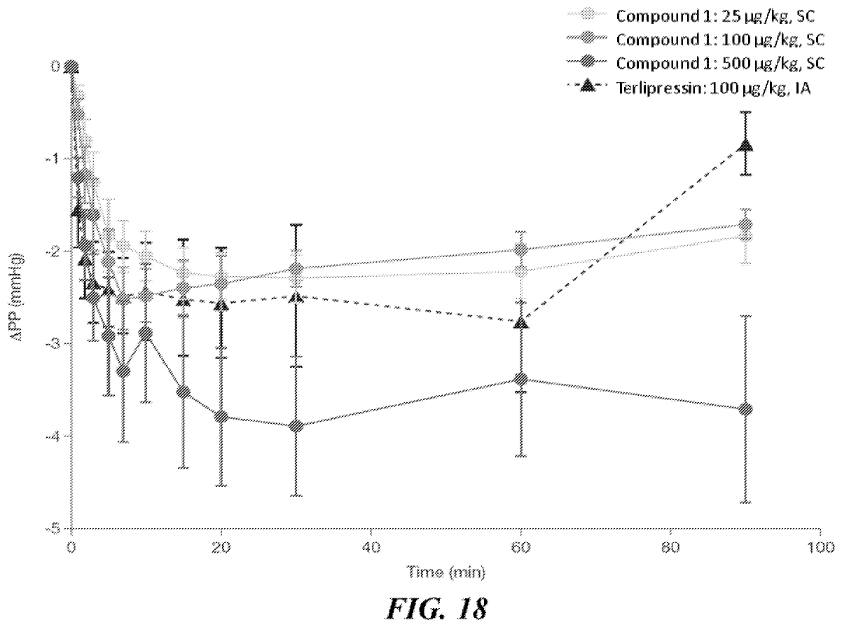

In some embodiments, such as described in the studies provided in Example 6], a compound described herein (e.g., a mixed V1AR agonist-antagonist, such as Compound 1) decreases portal pressure (PP) (after subcutaneous administration) without excessive vasoconstriction, such as over a wide dose range (e.g., 10 μg/kg to 500 μg/kg of Compound 1), see FIGS. 16 and 18. In some embodiments, such as described in the studies provided in Example 6, a compound described herein (e.g., a mixed V1AR agonist-antagonist, such as Compound 1) decreases PP (after subcutaneous administration), such as over a wide dose range (e.g., 10 μg/kg to 500 μg/kg of Compound 1), see FIGS. 16 and 18.

In some embodiments, a method provided herein further comprises evaluating a biological sample (e.g., of an individual). In some embodiments, the method further comprises evaluating a biological sample of an individual for a biomarker, such as a biomarker described in Example 6. In some embodiments, the biomarker (e.g., the amount or level of the biomarker) is compared to a control or a standard (e.g., amount or level of the biomarker).

In some embodiments, a biomarker described herein, such as a biomarker described in Example 6, is evaluated at a first time point and a second time point. In some embodiments, the second time point is used to determine responsiveness or efficacy, such as of a compound described herein (e.g., a mixed V1AR agonist-antagonist, such as Compound 1). In some embodiments, the second time point is an endpoint. In some instances, the endpoint is used to determine an amount or level of a biomarker sufficient to achieve the desired responsiveness or efficacy of a compound described herein (e.g., a mixed V1AR agonist-antagonist, such as Compound 1). In some embodiments, a method provided herein further comprises administering a compound described herein (e.g., a mixed V1AR agonist-antagonist, such as Compound 1) to an individual at least until the level or amount of a biomarker (of the individual) reaches the endpoint. In some embodiments, the method comprises continuing to administer the compound after the level or amount of a biomarker (of the individual) reaches the endpoint.

In some embodiments, a level or amount of a biomarker described herein, such as a biomarker described in Example 6, is higher (e.g., at least 5% higher, at least 15% higher, at least 25% higher, at least 35% higher, at least 45% higher, at least 55% higher, at least 65% higher, at least 75% higher, at least 85% higher, at least 95% higher, or more) at the second timepoint compared to the first timepoint.

In some embodiments, a level or amount of a biomarker described herein, such as a biomarker described in Example 6, is lower (e.g., at most 95% lower, at most 85% lower, at most 75% lower, at most 65% lower, at most 55% lower, at most 45% lower, at most 35% lower, at most 25% lower, at most 15% lower, at most 5% lower, or less) at the second timepoint compared to the first timepoint.

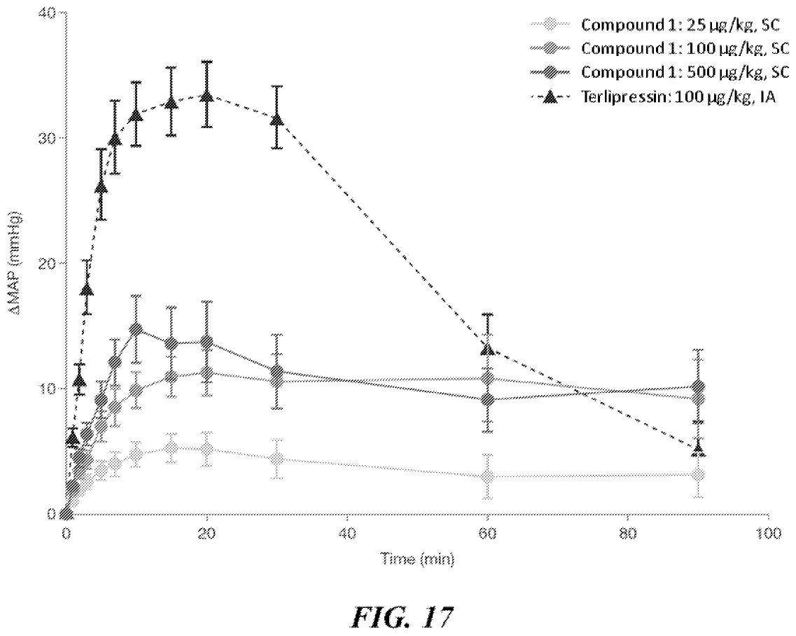

In some embodiments, such as described in the studies provided in Example 6, a compound described herein (e.g., a mixed V1AR agonist-antagonist, such as Compound 1) increases mean arterial pressure (MAP) (after subcutaneous administration), such as over a wide dose range (e.g., 10 μg/kg to 500 μg/kg of Compound 1), see FIG. 17. In some embodiments, such as described in the studies provided in Example 6, the increase in MAP reaches a peak plateau or a therapeutic ceiling (e.g., an increase in MAP of about 10 to about 15 mmHg), see FIG. 17. In some embodiments, such as described in the studies provided in Example 6, the increase in MAP reaches a peak plateau or a therapeutic ceiling (e.g., an increase in MAP of about 10 to about 15 mmHg) alongside a decrease in PP (of about 2 to about 14 mmHg), see FIG. 17. In some instances, even after a 5-fold increase in dose (e.g., 100 μg/kg or 500 μg/kg of Compound 1), a compound described herein does not produce a meaningful further change in an effect, such as an increase in MA, see FIG. 17.

In other instances, administration of a full V1a receptor agonist, such as terlipressin, provide a markedly higher increases in MAP (and beyond a treatment window of 10-15 mmHg), such as despite reductions in PP being similar to those for a compound described herein (e.g., a mixed V1AR agonist-antagonist, such as Compound 1), see FIG. 17.

Provided in some embodiments herein are methods of treating complications of end-stage liver disease, such as, HRS-AKI, in an individual (e.g., in need thereof), the method comprising administering a therapeutically effective amount of a compound described herein (e.g., Compound 1), or a pharmaceutically acceptable salt (e.g., an acetate salt), to the individual.

In some embodiments, an individual described herein has cirrhotic portal hypertension. In some embodiments, an individual described herein end-stage liver disease (ESLD). In some embodiments, an individual described herein has HRS-AKI. In some embodiments, an individual described herein has developed HRS-AKI as a complication of ESLD.

In some embodiments, a compound described herein (e.g., Compound 1) provides a substantially improved therapeutic index (e.g., arising from a lower maximal vasoconstrictive effect and lower risk for tissue hypoxia), such as, when compared to full V1A receptor agonists. In some embodiments, a compound described herein (e.g., Compound 1) provides about half of the maximal vasoconstriction produced by full agonists, such as, without any concomitant signs of ischemia. In some embodiments, a compound described herein (e.g., Compound 1) is a (clinically) efficacious vasoconstrictor (e.g., having a favorable benefit/risk profile, such as, having low to no organ toxicities).

Provided in some embodiments herein is a method of treating hepatorenal syndrome with acute kidney injury (HRS-AKI) in an individual (e.g., in need thereof), the method comprising administering a therapeutically effective amount of a compound described herein (e.g., Compound 1), or a pharmaceutically acceptable salt (e.g., an acetate salt), to the individual.

Provided in some embodiments herein is a method of treating hepatorenal syndrome with acute kidney injury (HRS-AKI) in an individual, such as an individual who has developed HRS-AKI as a complication of cirrhosis with ascites, the method comprising administering a therapeutically effective amount of a compound described herein (e.g., Compound 1), or a pharmaceutically acceptable salt (e.g., an acetate salt), to the individual.

In some embodiments, the individual has liver disease (ESLD). In some embodiments, the individual has HRS-AKI as a complication of end-stage liver disease (ESLD). In some embodiments, the individual has developed HRS-AKI as a complication of end-stage liver disease (ESLD).

In some embodiments, the individual has (e.g. decompensated) cirrhosis. In some embodiments, the individual has decompensated cirrhosis. In some embodiments, the individual has decompensated cirrhosis with ascites.

In some embodiments, the therapeutically effective amount of a compound described herein (e.g., Compound 1), or the pharmaceutically acceptable salt (e.g., the acetate salt), is about 5 μg to about 55 μg. In some embodiments, the therapeutically effective amount of a compound described herein (e.g., Compound 1), or the pharmaceutically acceptable salt (e.g., the acetate salt), is about 8 μg to about 50 μg. In some embodiments, the therapeutically effective amount of a compound described herein (e.g., Compound 1), or the pharmaceutically acceptable salt (e.g., the acetate salt), is about 20 μg to about 35 μg. In some embodiments, the therapeutically effective amount of a compound described herein (e.g., Compound 1), or the pharmaceutically acceptable salt (e.g., the acetate salt), is about 25 μg to about 35 μg. In some embodiments, the therapeutically effective amount of a compound described herein (e.g., Compound 1), or the pharmaceutically acceptable salt (e.g., the acetate salt), is about 30 μg. In some embodiments, the therapeutically effective amount of a compound described herein (e.g., Compound 1), or the pharmaceutically acceptable salt (e.g., the acetate salt), is administered over a period of time, such as over several hours (e.g., continuously for up to 24-hr) for several days (e.g., up to 10 days).

In some embodiments, a compound described herein (e.g., Compound 1), or the pharmaceutically acceptable salt (e.g., the acetate salt), is administered to the individual intravenously. In some embodiments, Compound 1, or the pharmaceutically acceptable salt (e.g., the acetate salt), is administered to the individual by intravenous infusion.

In some embodiments, a compound described herein (e.g., Compound 1), or the pharmaceutically acceptable salt (e.g., the acetate salt), is administered to the individual subcutaneously. In some embodiments, Compound 1, or the pharmaceutically acceptable salt (e.g., the acetate salt), is administered to the individual by subcutaneous injection.

In some embodiments, the therapeutically effective amount of a compound described herein (e.g., Compound 1), or the pharmaceutically acceptable salt (e.g., the acetate salt), is about 5 μg/hr or more. In some embodiments, the therapeutically effective amount of a compound described herein (e.g., Compound 1), or the pharmaceutically acceptable salt (e.g., the acetate salt), is about 55 μg/hr or less. In some embodiments, the therapeutically effective amount of a compound described herein (e.g., Compound 1), or the pharmaceutically acceptable salt (e.g., the acetate salt), is about 5 μg/hr to about 55 μg/hr. In some embodiments, the therapeutically effective amount of a compound described herein (e.g., Compound 1), or the pharmaceutically acceptable salt (e.g., the acetate salt), is about 8 μg/hr to about 50 μg/hr. In some embodiments, the therapeutically effective amount of a compound described herein (e.g., Compound 1), or the pharmaceutically acceptable salt (e.g., the acetate salt), is about 20 μg/hr to about 35 μg/hr. In some embodiments, the therapeutically effective amount of a compound described herein (e.g., Compound 1), or the pharmaceutically acceptable salt (e.g., the acetate salt), is about 25 μg/hr to about 35 μg/hr. In some embodiments, the therapeutically effective amount of a compound described herein (e.g., Compound 1), or the pharmaceutically acceptable salt (e.g., the acetate salt), is about 30 μg/hr.

In some embodiments, a compound described herein (e.g., Compound 1), or the pharmaceutically acceptable salt (e.g., the acetate salt), is administered to the individual on a first day and a second day. In some embodiments, a compound described herein (e.g., Compound 1), or the pharmaceutically acceptable salt (e.g., the acetate salt), is (e.g., subcutaneously) administered to the individual one or more days after the first day. In some embodiments, the individual receives an initial (e.g., intravenous infusion) dose of a compound described herein (e.g., Compound 1), or the pharmaceutically acceptable salt (e.g., the acetate salt), on the first day. In some embodiments, the individual receives an initial (e.g., intravenous infusion) dose of a compound described herein (e.g., Compound 1), or the pharmaceutically acceptable salt (e.g., the acetate salt), on the first day to acclimate the individual to vasoconstriction before receiving a first assigned (e.g., subcutaneous) treatment dose, such as on the second day.

In some embodiments, a compound provided herein is administered to an individual (e.g., in need thereof) in an amount of about 0.01 milligrams (mg) or more. In some embodiments, a compound provided herein is administered to an individual (e.g., in need thereof) in an amount of about 0.1 mg or more. In some embodiments, a compound provided herein is administered to an individual (e.g., in need thereof) in an amount of about 0.3 mg or more. In some embodiments, a compound provided herein is administered to an individual (e.g., in need thereof) in an amount of about 0.45 mg or more. In some embodiments, a compound provided herein is administered to an individual (e.g., in need thereof) in an amount of about 0.6 mg or more. In some embodiments, a compound provided herein is administered to an individual (e.g., in need thereof) in an amount of about 0.9 mg or more. In some embodiments, a compound provided herein is administered to an individual (e.g., in need thereof) in an amount of about 10 mg or more. In some embodiments, a compound provided herein is administered to an individual (e.g., in need thereof) in an amount of about 20 mg or more. In some embodiments, a compound provided herein is administered to an individual (e.g., in need thereof) in an amount of about 30 mg or more. In some embodiments, a compound provided herein is administered to an individual (e.g., in need thereof) in an amount of about 40 mg or more. In some embodiments, a compound provided herein is administered to an individual (e.g., in need thereof) in an amount of about 50 mg or more. In some embodiments, a compound provided herein is administered to an individual (e.g., in need thereof) in an amount of about 0.01 mg to about 50 mg. In some embodiments, a compound provided herein is administered to an individual (e.g., in need thereof) in an amount of about 0.01 mg to about 10 mg. In some embodiments, a compound provided herein is administered to an individual (e.g., in need thereof) in an amount of about 0.01 mg to about 1 mg.

In some embodiments, the initial (e.g., intravenous infusion) dose is about 0.01 milligrams (mg) or more. In some embodiments, the initial (e.g., intravenous infusion) dose is about 0.1 mg or more. In some embodiments, the initial (e.g., intravenous infusion) dose is about 0.3 mg or more. In some embodiments, the initial (e.g., intravenous infusion) dose is about 0.45 mg or more. In some embodiments, the initial (e.g., intravenous infusion) dose is about 0.6 mg or more. In some embodiments, the initial (e.g., intravenous infusion) dose is about 0.9 mg or more. In some embodiments, the initial (e.g., intravenous infusion) dose is about 10 mg or more. In some embodiments, the initial (e.g., intravenous infusion) dose is about 20 mg or more. In some embodiments, the initial (e.g., intravenous infusion) dose is about 30 mg or more. In some embodiments, the initial (e.g., intravenous infusion) dose is about 40 mg or more. In some embodiments, the initial (e.g., intravenous infusion) dose is about 50 mg or more. In some embodiments, the initial (e.g., intravenous infusion) dose is about 0.01 mg to about 50 mg. In some embodiments, the initial (e.g., intravenous infusion) dose is about 0.01 mg to about 10 mg. In some embodiments, the initial (e.g., intravenous infusion) dose is about 0.01 mg to about 1 mg.

In some embodiments, a dose (e.g., subcutaneously) administered subsequent to the initial dose is about 0.01 milligrams (mg) or more. In some embodiments, a dose (e.g., subcutaneously) administered subsequent to the initial dose is about 0.1 mg or more. In some embodiments, a dose (e.g., subcutaneously) administered subsequent to the initial dose is about 0.3 mg or more. In some embodiments, a dose (e.g., subcutaneously) administered subsequent to the initial dose is about 0.45 mg or more. In some embodiments, a dose (e.g., subcutaneously) administered subsequent to the initial dose is about 0.6 mg or more. In some embodiments, a dose (e.g., subcutaneously) administered subsequent to the initial dose is about 0.9 mg or more. In some embodiments, a dose (e.g., subcutaneously) administered subsequent to the initial dose is about 10 mg or more. In some embodiments, a dose (e.g., subcutaneously) administered subsequent to the initial dose is about 20 mg or more. In some embodiments, a dose (e.g., subcutaneously) administered subsequent to the initial dose is about 30 mg or more. In some embodiments, a dose (e.g., subcutaneously) administered subsequent to the initial dose is about 40 mg or more. In some embodiments, a dose (e.g., subcutaneously) administered subsequent to the initial dose is about 50 mg or more. In some embodiments, a dose (e.g., subcutaneously) administered subsequent to the initial dose is about 0.01 mg to about 50 mg. In some embodiments, a dose (e.g., subcutaneously) administered subsequent to the initial dose is about 0.01 mg to about 10 mg. In some embodiments, a dose (e.g., subcutaneously) administered subsequent to the initial dose is about 0.01 mg to about 1 mg.

In some embodiments, the initial (e.g., intravenous infusion) dose of a compound described herein (e.g., Compound 1), or the pharmaceutically acceptable salt (e.g., the acetate salt), is a low dose. In some embodiments, the initial (e.g., intravenous infusion) dose of a compound described herein (e.g., Compound 1), or the pharmaceutically acceptable salt (e.g., the acetate salt), is a dose of about 5 μg/hr to about 15 μg/hr. In some embodiments, the initial (e.g., intravenous infusion) dose of a compound described herein (e.g., Compound 1), or the pharmaceutically acceptable salt (e.g., the acetate salt), is a dose of about 8 μg/hr.

In some embodiments, a compound described herein (e.g., Compound 1), or the pharmaceutically acceptable salt (e.g., the acetate salt), is administered to the individual on the first day (e.g., by intravenous infusion) for a period of about 4 hr to about 8 hr. In some embodiments, a compound described herein (e.g., Compound 1), or the pharmaceutically acceptable salt (e.g., the acetate salt), is administered to the individual on the first day (e.g., by intravenous infusion) for a period of about 6 hr.

In some embodiments, a first dose of the compound, or the pharmaceutically acceptable salt thereof, is administered to the individual on the first day, and a second dose is administered to the individual of the second day. In some embodiments, the first dose and the second dose comprise the same amount of the compound or the pharmaceutically acceptable salt thereof.

In some embodiments, a compound, or a pharmaceutically acceptable salt thereof, described herein (e.g., Compound 1) is (e.g., subcutaneously) administered to an individual (e.g., described herein) in an amount of about 0.01 milligrams (mg)/day or more. In some embodiments, a compound, or a pharmaceutically acceptable salt thereof, described herein (e.g., Compound 1) is (e.g., subcutaneously) administered to an individual (e.g., described herein) in an amount of about 0.1 mg/day or more. In some embodiments, a compound, or a pharmaceutically acceptable salt thereof, described herein (e.g., Compound 1) is (e.g., subcutaneously) administered to an individual (e.g., described herein) in an amount of about 0.3 mg/day or more. In some embodiments, a compound, or a pharmaceutically acceptable salt thereof, described herein (e.g., Compound 1) is (e.g., subcutaneously) administered to an individual (e.g., described herein) in an amount of about 0.45 mg/day or more. In some embodiments, a compound, or a pharmaceutically acceptable salt thereof, described herein (e.g., Compound 1) is (e.g., subcutaneously) administered to an individual (e.g., described herein) in an amount of about 0.6 mg/day or more. In some embodiments, a compound, or a pharmaceutically acceptable salt thereof, described herein (e.g., Compound 1) is (e.g., subcutaneously) administered to an individual (e.g., described herein) in an amount of about 0.9 mg/day or more. In some embodiments, a compound, or a pharmaceutically acceptable salt thereof, described herein (e.g., Compound 1) is (e.g., subcutaneously) administered to an individual (e.g., described herein) in an amount of about 10 mg/day or more. In some embodiments, a compound, or a pharmaceutically acceptable salt thereof, described herein (e.g., Compound 1) is (e.g., subcutaneously) administered to an individual (e.g., described herein) in an amount of about 50 mg/day or more. In some embodiments, a compound, or a pharmaceutically acceptable salt thereof, described herein (e.g., Compound 1) is (e.g., subcutaneously) administered to an individual (e.g., described herein) in an amount of about 100 mg/day or more. In some embodiments, a compound, or a pharmaceutically acceptable salt thereof, described herein (e.g., Compound 1) is (e.g., subcutaneously) administered to an individual (e.g., described herein) in an amount of about 100 mg/day or less. In some embodiments, a compound, or a pharmaceutically acceptable salt thereof, described herein (e.g., Compound 1) is (e.g., subcutaneously) administered to an individual (e.g., described herein) in an amount of about 50 mg/day or less. In some embodiments, a compound, or a pharmaceutically acceptable salt thereof, described herein (e.g., Compound 1) is (e.g., subcutaneously) administered to an individual (e.g., described herein) in an amount of about 10 mg/day or less. In some embodiments, a compound, or a pharmaceutically acceptable salt thereof, described herein (e.g., Compound 1) is (e.g., subcutaneously) administered to an individual (e.g., described herein) in an amount of about 1 mg/day or less. In some embodiments, a compound, or a pharmaceutically acceptable salt thereof, described herein (e.g., Compound 1) is (e.g., subcutaneously) administered to an individual (e.g., described herein) in an amount of about 0.1 mg/day or less. In some embodiments, a compound, or a pharmaceutically acceptable salt thereof, described herein (e.g., Compound 1) is (e.g., subcutaneously) administered to an individual (e.g., described herein) in an amount of about 0.01 mg/day to about 100 mg/day. In some embodiments, a compound, or a pharmaceutically acceptable salt thereof, described herein (e.g., Compound 1) is (e.g., subcutaneously) administered to an individual (e.g., described herein) in an amount of about 0.01 mg/day to about 10 mg/day. In some embodiments, a compound, or a pharmaceutically acceptable salt thereof, described herein (e.g., Compound 1) is (e.g., subcutaneously) administered to an individual (e.g., described herein) in an amount of about 0.01 mg/day to about 1 mg/day.

In some embodiments, a compound described herein (e.g., Compound 1), or the pharmaceutically acceptable salt (e.g., the acetate salt), is (e.g., subcutaneously) administered to the individual on multiple days (e.g., for 5 days). In some embodiments, the method comprises (e.g., subcutaneously) administering a compound described herein (e.g., Compound 1), or the pharmaceutically acceptable salt (e.g., the acetate salt), to the individual on one or more days. In some embodiments, a compound described herein (e.g., Compound 1), or the pharmaceutically acceptable salt (e.g., the acetate salt), is (e.g., subcutaneously) administered to the individual on subsequent days. In some embodiments, the method comprises (e.g., subcutaneously) administering a compound described herein (e.g., Compound 1), or the pharmaceutically acceptable salt (e.g., the acetate salt), to the individual on consecutive and/or non-consecutive days. In some embodiments, the method comprises (e.g., subcutaneously) administering a compound described herein (e.g., Compound 1), or the pharmaceutically acceptable salt (e.g., the acetate salt), to the individual on one or more consecutive days. In some embodiments, the method comprises (e.g., subcutaneously) administering a compound described herein (e.g., Compound 1), or the pharmaceutically acceptable salt (e.g., the acetate salt), to the individual on one or more non-consecutive days. In some embodiments, the method comprises (e.g., subcutaneously) administering a compound described herein (e.g., Compound 1), or the pharmaceutically acceptable salt (e.g., the acetate salt), to the individual on one or more consecutive days after the first day. In some embodiments, the method comprises (e.g., subcutaneously) administering a compound described herein (e.g., Compound 1), or the pharmaceutically acceptable salt (e.g., the acetate salt), to the individual on one or more non-consecutive days after the first day.

In some embodiments, the method comprises (e.g., subcutaneously) administering a compound described herein (e.g., Compound 1), or the pharmaceutically acceptable salt (e.g., the acetate salt), to the individual for at least two days. In some embodiments, the method comprises (e.g., subcutaneously) administering a compound described herein (e.g., Compound 1), or the pharmaceutically acceptable salt (e.g., the acetate salt), to the individual for five or more days. In some embodiments, the method comprises (e.g., subcutaneously) administering a compound described herein (e.g., Compound 1), or the pharmaceutically acceptable salt (e.g., the acetate salt), to the individual for ten or more days.

In some embodiments, the method comprises (e.g., subcutaneously) administering a compound described herein (e.g., Compound 1), or the pharmaceutically acceptable salt (e.g., the acetate salt), to the individual for a week or more.

In some embodiments, the method comprises (e.g., subcutaneously) administering a compound described herein (e.g., Compound 1), or the pharmaceutically acceptable salt (e.g., the acetate salt), to the individual for a month or more.

In some embodiments, the method comprises (e.g., subcutaneously) administering a compound described herein (e.g., Compound 1), or the pharmaceutically acceptable salt (e.g., the acetate salt), to the individual for a year or more.

In some embodiments, the method comprises (e.g., subcutaneously) administering a compound described herein (e.g., Compound 1), or the pharmaceutically acceptable salt (e.g., the acetate salt), to the individual for up to 11 days, such as for up to 10 days after the first day. In some embodiments, the method comprises (e.g., subcutaneously) administering a compound described herein (e.g., Compound 1), or the pharmaceutically acceptable salt (e.g., the acetate salt), to the individual for 4 to 10 days. In some embodiments, the method comprises (e.g., subcutaneously) administering a compound described herein (e.g., Compound 1), or the pharmaceutically acceptable salt (e.g., the acetate salt), to the individual for 4 to 10 days after the first day. In some embodiments, the method comprises (e.g., subcutaneously) administering a compound described herein (e.g., Compound 1), or the pharmaceutically acceptable salt (e.g., the acetate salt), to the individual for up to 9 days. In some embodiments, the days are consecutive days. In some embodiments, the days are non-consecutive days. In some embodiments, the method comprises administering a compound described herein (e.g., Compound 1), or the pharmaceutically acceptable salt (e.g., the acetate salt), to the individual (e.g., via continuous intravenous infusion) over a 24-hr period.

In some embodiments, the method comprises administering a compound described herein (e.g., Compound 1), or the pharmaceutically acceptable salt (e.g., the acetate salt), to the individual via continuous intravenous infusion over a 24-hr period per day (for up to 9 days). In some embodiments, the method comprises subcutaneously administering a compound described herein (e.g., Compound 1), or the pharmaceutically acceptable salt (e.g., the acetate salt), to the individual (e.g., for up to 5 days). In some embodiments, a compound described herein (e.g., Compound 1), or the pharmaceutically acceptable salt (e.g., the acetate salt), is administered to the individual in an amount of up to about 2 milligrams (mg)/day. In some embodiments, a compound described herein (e.g., Compound 1), or the pharmaceutically acceptable salt (e.g., the acetate salt), is administered to the individual in an amount of about 0.2 mg/day to about 2 mg/day. In some embodiments, a compound described herein (e.g., Compound 1), or the pharmaceutically acceptable salt (e.g., the acetate salt), is administered to the individual in an amount of about 0.5 mg/day to about 1.5 mg/day. In some embodiments, a compound described herein (e.g., Compound 1), or the pharmaceutically acceptable salt (e.g., the acetate salt), is administered to the individual in an amount of about 1.2 mg/day.

In some embodiments, an individual (e.g., described herein) receives repeated subcutaneous injections of a compound, or the pharmaceutically acceptable salt thereof, described herein (e.g., Compound 1). In some embodiments, a compound, or the pharmaceutically acceptable salt thereof, described herein (e.g., Compound 1) is subcutaneously administered to an individual (e.g., described herein) once-daily. In some embodiments, a compound, or the pharmaceutically acceptable salt thereof, described herein (e.g., Compound 1) is subcutaneously administered to an individual (e.g., described herein) once-daily for two or more consecutive days. In some embodiments, a compound, or the pharmaceutically acceptable salt thereof, described herein (e.g., Compound 1) is subcutaneously administered to an individual (e.g., described herein) once-daily for three or more consecutive days. In some embodiments, a compound, or the pharmaceutically acceptable salt thereof, described herein (e.g., Compound 1) is subcutaneously administered to an individual (e.g., described herein) once-daily for four or more consecutive days. In some embodiments, a compound, or the pharmaceutically acceptable salt thereof, described herein (e.g., Compound 1) is subcutaneously administered to an individual (e.g., described herein) once-daily for five or more consecutive days.

In some embodiments, In some embodiments, a compound, or the pharmaceutically acceptable salt thereof, described herein (e.g., Compound 1) is administered to an individual (e.g., described herein) via subcutaneous bolus injection.

In some embodiments, In some embodiments, a compound, or the pharmaceutically acceptable salt thereof, described herein (e.g., Compound 1) is administered to an individual (e.g., described herein) via subcutaneous infusion. In some embodiments, a compound, or the pharmaceutically acceptable salt thereof, described herein (e.g., Compound 1) is administered to an individual (e.g., described herein) via continuous subcutaneous infusion.

Unless stated otherwise, a composition for subcutaneous administration means a composition, that when administered subcutaneously, is suitable for systemic delivery of the active agent or API (e.g., Compound 1).

Unless stated otherwise, formulated means a composition comprising an excipient, such as a stabilizing agent or a diluent.

Unless stated otherwise, weights (e.g., doses) provided herein for a compound described herein (e.g., Compound 1) are calculated based-on the free base of the compound (e.g., not a pharmaceutically acceptable salt of the compound). In some instances, the acetate salt of a compound described herein (e.g., Compound 1) is administered to the individual receiving a treatment provided herein.

In some instances, a compound described herein (e.g., a mixed V1AR agonist-antagonist, such as Compound 1) has an effect (e.g., increase or decrease) on the level of a biomarker described herein, such as a biomarker described in Examples 5 or 6. In some embodiments, a biomarker described herein is selected from the group consisting of mean arterial pressure (MAP), (plasma) aldosterone, (plasma) renin, ascites volume, body weight, urine volume, (net) fluid balance, urine sodium, urine potassium, portal pressure, cardiac output, systemic vascular resistance, BUN, BUN/CREA, PHOS, spleen weight, skin blood flow (SBF), blood lactate concentration, heart rate, systolic arterial pressure, diastolic arterial pressure, and blood pH. In some embodiments, a biomarker described herein is mean arterial pressure (MAP). In some embodiments, a biomarker described herein is aldosterone. In some embodiments, a biomarker described herein is renin. In some embodiments, a biomarker described herein is ascites volume. In some embodiments, a biomarker described herein is body weight. In some embodiments, a biomarker described herein is urine volume. In some embodiments, a biomarker described herein is net fluid balance. In some embodiments, a biomarker described herein is urine sodium. In some embodiments, a biomarker described herein is urine potassium. In some embodiments, a biomarker described herein is spleen weight. In some embodiments, a biomarker described herein is systolic arterial pressure. In some embodiments, a biomarker described herein is diastolic arterial pressure. In some embodiments, a biomarker described herein is heart rate. In some embodiments, a biomarker described herein is portal pressure. In some embodiments, a biomarker described herein is skin blood flow (SBF). In some embodiments, a biomarker described herein is blood lactate concentration. In some embodiments, a biomarker described herein is heart rate. In some embodiments, a biomarker described herein is systolic arterial pressure. In some embodiments, a biomarker described herein is diastolic arterial pressure.

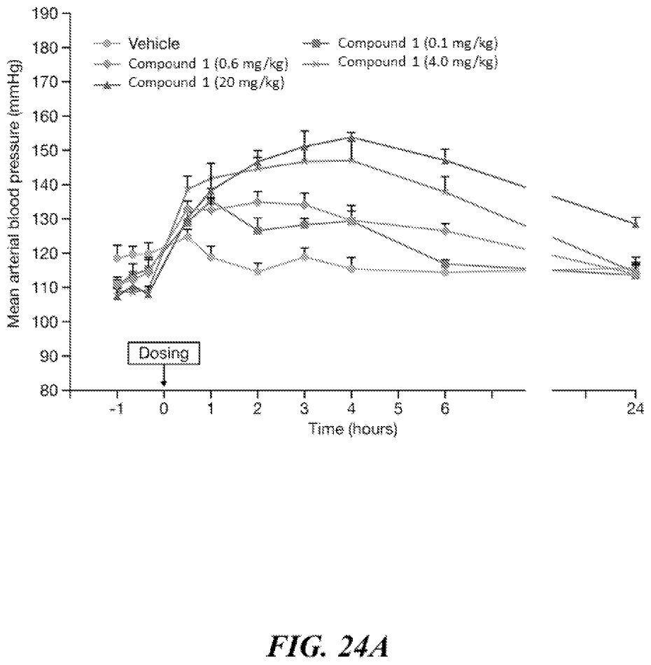

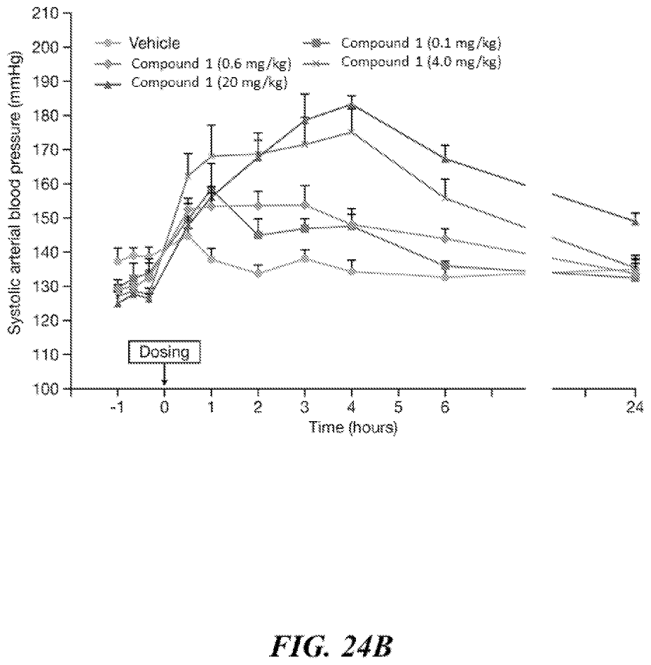

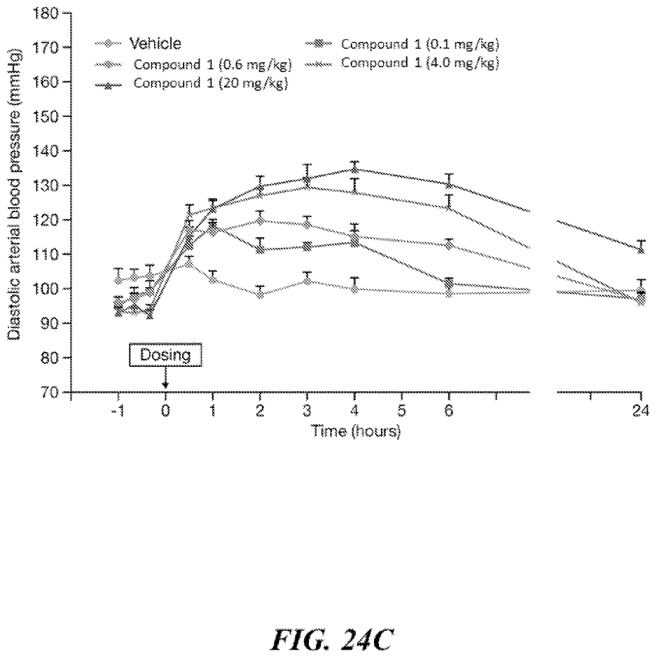

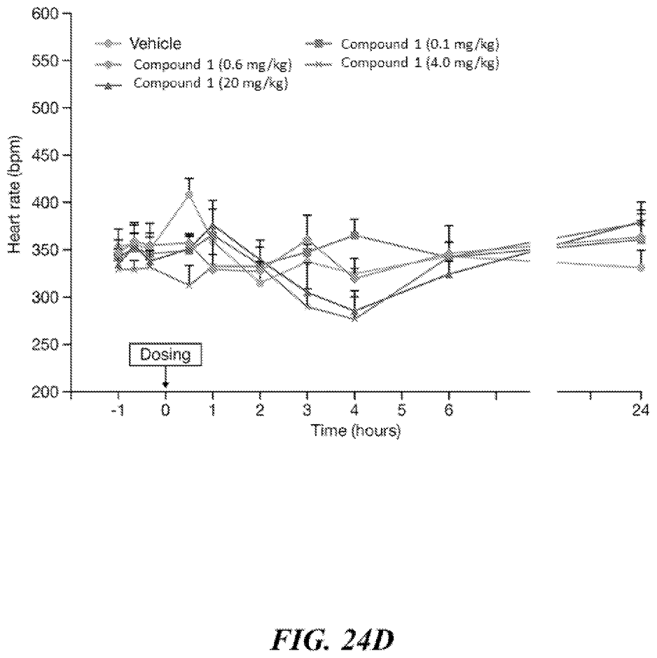

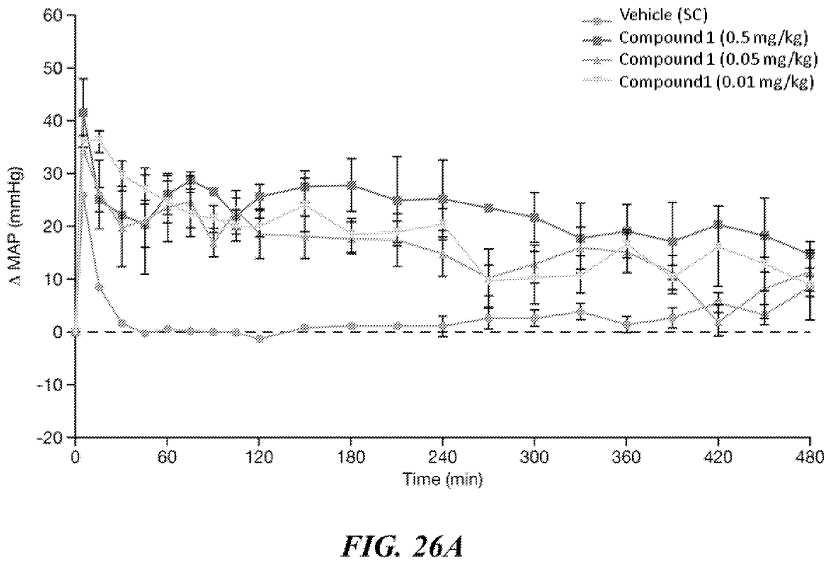

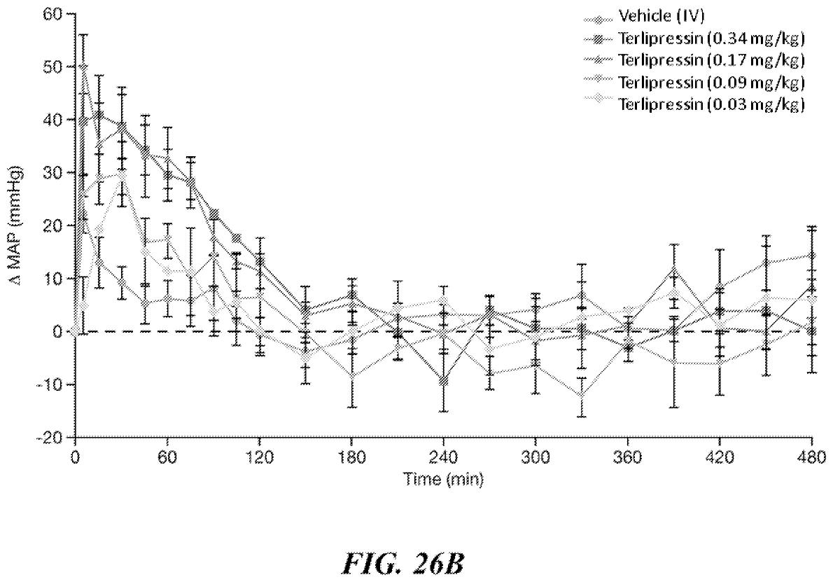

In some embodiments, a compound described herein (e.g., a mixed V1AR agonist-antagonist, such as Compound 1) increases mean arterial blood pressure, systolic arterial blood pressure, and diastolic arterial blood pressure in an individual (e.g., a mammal), such as after subcutaneous administration (e.g., see FIG. 24A-C). In some instances, the occurrence and amplitude of maximum increase in blood pressure is related to dose of a compound described herein (e.g., a mixed V1AR agonist-antagonist, such as Compound 1) in an individual (e.g., a mammal), such as after subcutaneous administration. In some embodiments, a compound described herein (e.g., a mixed V1AR agonist-antagonist, such as Compound 1) increases mean arterial blood pressure (MAP) by at least about 10 mmHg (e.g., at least 15 mmHg, at least 20 mmHg, at least 30 mmHg, at least 40 mmHg) in an individual (e.g., a mammal), such as after subcutaneous administration (e.g., see FIG. 26A). In some embodiments, a compound described herein (e.g., a mixed V1AR agonist-antagonist, such as Compound 1) increases mean arterial blood pressure (MAP) by at most about 50 mmHg (e.g., at most 40 mmHg, at most 30 mmHg, at most 20 mmHg, at most 10 mmHg) in an individual (e.g., a mammal), such as after subcutaneous administration. In some embodiments, a compound described herein (e.g., a mixed V1AR agonist-antagonist, such as Compound 1) increases mean arterial blood pressure (MAP) by about 10 mmHg to about 50 mmHg in an individual (e.g., a mammal), such as after subcutaneous administration. In some embodiments, a compound described herein (e.g., a mixed V1AR agonist-antagonist, such as Compound 1) increases mean arterial blood pressure (MAP) by about 20 mmHg in an individual (e.g., a mammal), such as after subcutaneous administration. In some embodiments, a compound described herein (e.g., a mixed V1AR agonist-antagonist, such as Compound 1) increases mean arterial blood pressure (MAP) by about 40 mmHg in an individual (e.g., a mammal), such as after subcutaneous administration. In some embodiments, a compound described herein (e.g., a mixed V1AR agonist-antagonist, such as Compound 1) increases mean arterial blood pressure (MAP) by about 25 mmHg in an individual (e.g., a mammal), such as after subcutaneous administration. In some embodiments, a compound described herein (e.g., a mixed V1AR agonist-antagonist, such as Compound 1) increases and sustains the increase in mean arterial blood pressure (MAP) for at least 75 minutes (e.g., at least 150 minutes, at least 300 minutes) in an individual (e.g., a mammal), such as after subcutaneous administration. In some instances, a compound described herein (e.g., a mixed V1AR agonist-antagonist, such as Compound 1) provides a change in mean arterial blood pressure (MAP) that is statistically different from vehicle in an individual (e.g., a mammal), such as after subcutaneous administration. In some embodiments, a compound described herein (e.g., a mixed V1AR agonist-antagonist, such as Compound 1) increases MAP and MAP does not return to baseline values in an individual (e.g., a mammal), such as after subcutaneous administration.

In some embodiments, a full antagonist, such as terlipressin, increases mean arterial pressure (MAP) in an individual (e.g., a mammal) (e.g., see FIG. 26B). In some embodiments, a full antagonist, such as terlipressin, increases mean arterial pressure (MAP) and the effect is only sustained for a short period of time, such as less than 90 minutes (e.g., no longer than 30, 75, or 90 minutes) after intravenous administration (e.g., see FIG. 26B). In some embodiments, a full antagonist, such as terlipressin, provides transient effects on MAP (e.g., see FIG. 26B). In some embodiments, a compound described herein (e.g., a mixed V1AR agonist-antagonist, such as Compound 1) provides an increase in MAP spanning a 50-fold dose range in an individual (e.g., a mammal), which in some instances, may represent the maximal effect on arterial pressure.

In some embodiments, a compound described herein (e.g., a mixed V1AR agonist-antagonist, such as Compound 1) decreases heart rate in an individual (e.g., a mammal), such as following subcutaneous administration, see FIG. 24D. In some embodiments, a compound described herein (e.g., a mixed V1AR agonist-antagonist, such as Compound 1) decreases heart rate by at least 2% (e.g., at least 5%, at least 10%, at least 15%) in an individual (e.g., a mammal), such as after subcutaneous administration (e.g., see FIG. 24D). In some embodiments, a compound described herein (e.g., a mixed V1AR agonist-antagonist, such as Compound 1) decreases heart rate by at most 20% (e.g., at most 15%, at most 10%, at most 5%) in an individual (e.g., a mammal), such as after subcutaneous administration (e.g., see FIG. 24D). In some embodiments, a compound described herein (e.g., a mixed V1AR agonist-antagonist, such as Compound 1) decreases heart rate by about 5% in an individual (e.g., a mammal), such as after subcutaneous administration (e.g., see FIG. 24D). In some instances, a compound described herein (e.g., a mixed V1AR agonist-antagonist, such as Compound 1) provides changes in MAP that are correlate with measurable decreases in heart rate in an individual (e.g., a mammal), such as after subcutaneous administration (e.g., see FIG. 24D, 26A). In some instances, a compound described herein (e.g., a mixed V1AR agonist-antagonist, such as Compound 1), changes in systolic blood pressure and diastolic blood pressure correlate with changes in MAP in an individual (e.g., a mammal), such as after subcutaneous administration (e.g., see FIG. 24A-C).

In some embodiments, a compound described herein (e.g., a mixed V1AR agonist-antagonist, such as Compound 1) has a terminal half life (t_{1/2term}) of at least 50 minutes (e.g., at least 70 minutes, at least 90 minutes, at least 110 minutes, at least 130 minutes) in an individual (e.g., a mammal), such as after subcutaneous administration. In some embodiments, a compound described herein (e.g., a mixed V1AR agonist-antagonist, such as Compound 1) has a terminal half life (t_{1/2term}) of at most 150 minutes (e.g., at most 130 minutes, at most 110 minutes, at most 90 minutes, at most 70 minutes) in an individual (e.g., a mammal), such as after subcutaneous injection. In some embodiments, a compound described herein (e.g., a mixed V1AR agonist-antagonist, such as Compound 1) has a terminal half life (t_{1/2term}) of about 50 to about 150 minutes in an individual (e.g., a mammal), such as after subcutaneous injection. In some embodiments, a compound described herein (e.g., a mixed V1AR agonist-antagonist, such as Compound 1) has a terminal half life (t_{1/2term}) of about 110 minutes in an individual (e.g., a mammal), such as after subcutaneous injection. In some embodiments, a compound described herein (e.g., a mixed V1AR agonist-antagonist, such as Compound 1) has an elimination half life (t_{1/2elim}) of at least 1 minute (e.g., at least 5 minutes, at least 10 minutes, at least 15 minutes, at least 20 minutes) in an individual (e.g., a mammal), such as after intravenous administration. In some embodiments, a compound described herein (e.g., a mixed V1AR agonist-antagonist, such as Compound 1) has an elimination half life (t_{1/2elim}) of at most 60 minutes (e.g., at most 40 minutes, at most 20 minutes, at most 10 minutes) in an individual (e.g., a mammal). In some embodiments, a compound described herein (e.g., a mixed V1AR agonist-antagonist, such as Compound 1) has an elimination half life (t_{1/2elim}) of about 1 minute to about 40 minutes in an individual (e.g., a mammal). In some embodiments, ca compound described herein (e.g., a mixed V1AR agonist-antagonist, such as Compound 1) has an elimination half life (t_{1/2elim}) of about 20 minutes in an individual (e.g., a mammal). In some embodiments, the terminal half life (t_{1/2term}) of the compound after subcutaneous administration is greater than the elimination half life (t_{1/2elim}).

In some embodiments, a compound described herein (e.g., a mixed V1AR agonist-antagonist, such as Compound 1) has a bioavailability of at least 40% (e.g., at least 45%, at least 50%, at least 55%, at least 60%, at least 65%, at least 70%) in an individual (e.g., a mammal), such as after subcutaneous administration. In some embodiments, a compound described herein (e.g., a mixed V1AR agonist-antagonist, such as Compound 1) has a bioavailability of at most 75% (e.g., at most 70%, at most 60%, at most 50%, at most 40%) in an individual (e.g., a mammal). In some embodiments, a compound described herein (e.g., a mixed V1AR agonist-antagonist, such as Compound 1) has a bioavailability of about 40% to about 70% in an individual (e.g., a mammal). In some embodiments, a compound described herein (e.g., a mixed V1AR agonist-antagonist, such as Compound 1) has a bioavailability of about 60% in an individual (e.g., a mammal).

In some embodiments, a compound described herein (e.g., a mixed V1AR agonist-antagonist, such as Compound 1) has a clearance rate in an individual (e.g., a mammal) similar to the glomerular filtration rate (e.g., 5-15 mL/min/kg), such as after subcutaneous administration. In some embodiments, a compound described herein (e.g., a mixed V1AR agonist-antagonist, such as Compound 1) has as filtration rate of at least 5 mL/min/kg (e.g., at least 7 mL/min/kg, at least 10 mL/min/kg, at least 13 mL/min/kg). In some embodiments, a compound described herein (e.g., a mixed V1AR agonist-antagonist, such as Compound 1) has a filtration rate of at most 25 mL/min/kg (e.g., at most 22 mL/min/kg, at most 18 mL/min/kg, at most 15 mL/min/kg). In some embodiments, a compound described herein (e.g., a mixed V1AR agonist-antagonist, such as Compound 1) has a filtration rate of about 20 mL/min/kg. In some embodiments, a compound described herein (e.g., a mixed V1AR agonist-antagonist, such as Compound 1) has a filtration rate of about 10 mL/min/kg.

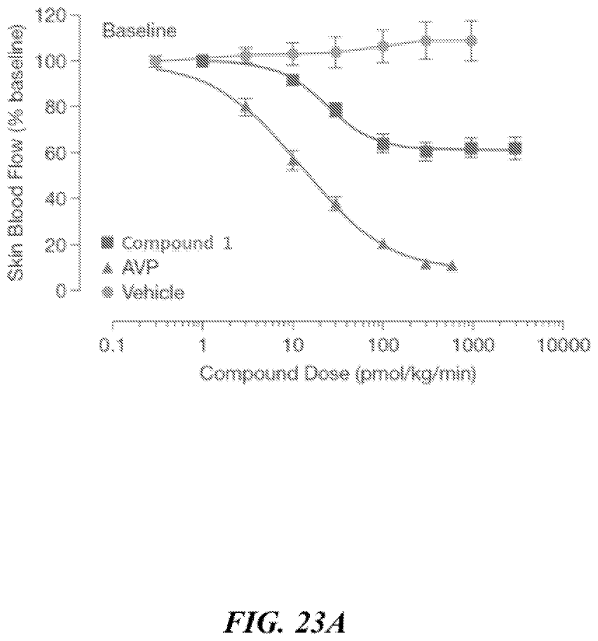

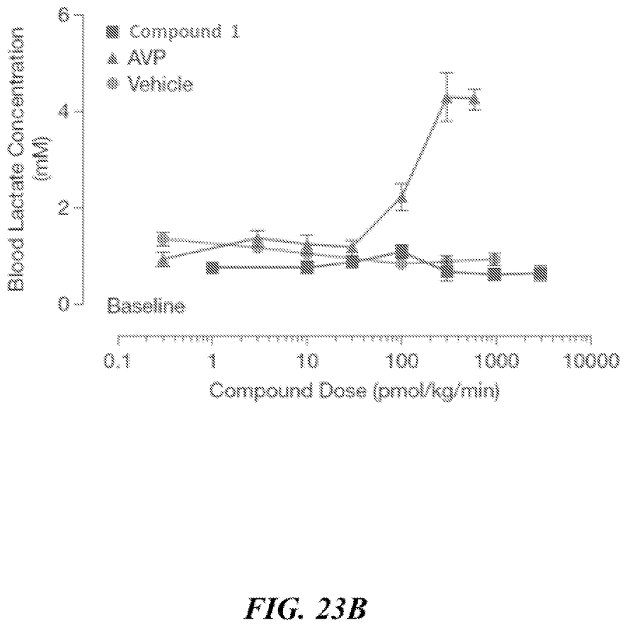

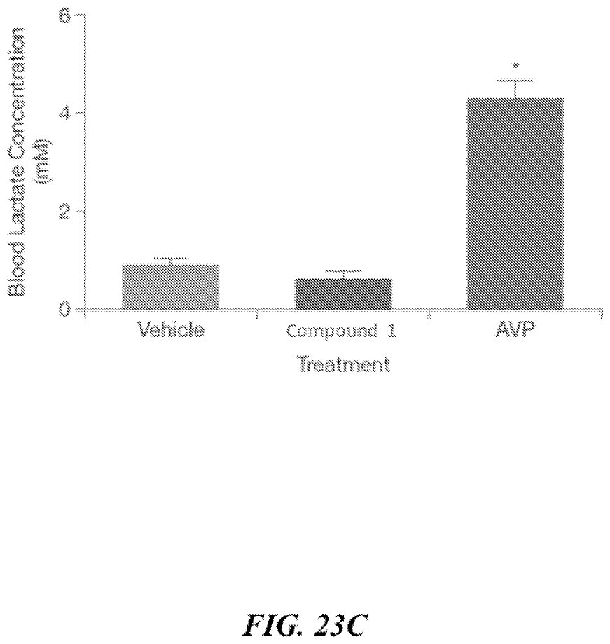

In some embodiments, a compound described herein (e.g., a mixed V1AR agonist-antagonist, such as Compound 1) decreases skin blood flow in an individual (e.g., a mammal), such as after intravenous administration (e.g., see FIG. 23A). In some embodiments, a compound described herein (e.g., a mixed V1AR agonist-antagonist, such as Compound 1) decreases skin blood flow in an individual (e.g., a mammal) by at most 60% (e.g., at most 55%, at most 50%, at most 45%, at most 40%). In some embodiments, a compound described herein (e.g., a mixed V1AR agonist-antagonist, such as Compound 1) decreases skin blood flow by at least 10% (e.g., at least 20%, at least 30%, at least 40%, at least 50%) in an individual (e.g., a mammal). In some embodiments, a compound described herein (e.g., a mixed V1AR agonist-antagonist, such as Compound 1) decreases skin blood flow by about 20% to about 60% in an individual (e.g., a mammal). In some embodiments, a compound described herein (e.g., a mixed V1AR agonist-antagonist, such as Compound 1) decreases skin blood flow by about 40% in an individual (e.g., a mammal). In some embodiments, a compound described herein (e.g., a mixed V1AR agonist-antagonist, such as Compound 1) decreases skin blood flow in an individual (e.g., a mammal) less than vasopressin decreases skin blood flow in an individual (e.g., a mammal), such as after intravenous administration, e.g., see FIG. 23A. In some instances, a compound described herein (e.g., a mixed V1AR agonist-antagonist, such as Compound 1) decreases skin blood flow by about 40% in an individual (e.g., a mammal) after intravenous administration and vasopressin decreases skin blood flow by about 90% in an individual (e.g., a mammal) after intravenous administration, demonstrating that a mixed V1AR agonist-antagonist described herein, such as Compound 1, is more suitable for intravenous administration than a full, nonselective (V2, V1a) agonist (e.g., vasopressin or terlipressin), such as by reducing (significant) injection site reactions (e.g., local vasoconstriction) that prevent a drug from being suitable for systemic delivery. In some instances, the lesser decrease in skin blood flow by a compound provided herein (e.g., a mixed V1AR agonist-antagonist, such as Compound 1) in comparison to vasopressin demonstrates a less local vasoconstriction in comparison to vasopressin.

In some instances, increased serum lactate levels are a clinical marker for anaerobic metabolism and tissue hypoxia and are used as a surrogate marker for the development of vasoconstriction and tissue ischemia. In some embodiments, a compound described herein (e.g., a mixed V1AR agonist-antagonist, such as Compound 1) does not (significantly) increase blood lactate concentration in an individual, such as after intravenous administration (e.g., see FIG. 23B). In some embodiments, a compound described herein (e.g., a mixed V1AR agonist-antagonist, such as Compound 1) does not (significantly) increase blood lactate levels while similar doses of vasopressin markedly increase blood lactate levels in an individual, such as after intravenous administration (e.g., see FIG. 23B-C). In some instances, vasopressin increases blood lactate levels by at least a 2-fold (e.g., a 3-fold, 4-fold) in an individual, such as after intravenous administration (e.g., see FIG. 23B).

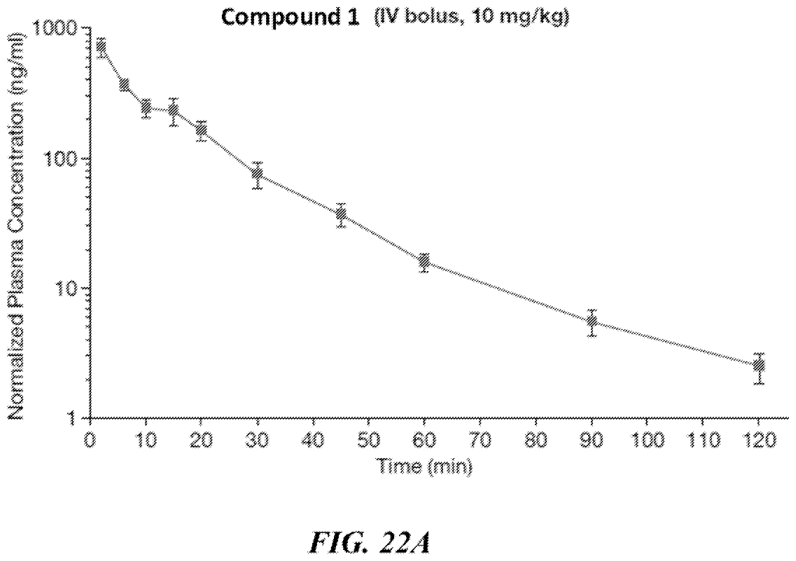

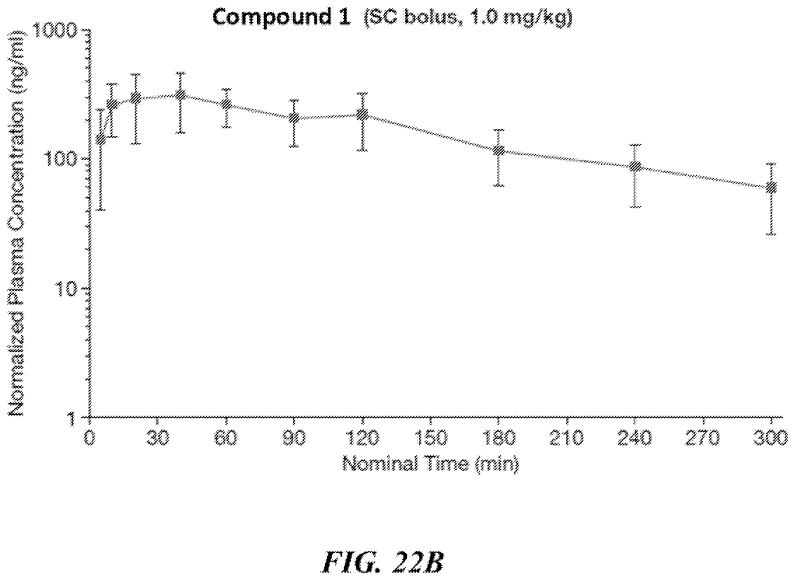

In some embodiments, a compound described herein (e.g., a mixed V1AR agonist-antagonist, such as Compound 1) decreases plasma concentration (e.g., linearly) (see e.g., FIG. 22A-B, 25A-B).

In some embodiments, a compound described herein (e.g., a mixed V1AR agonist-antagonist, such as Compound 1) increases blood pH in an individual, such as after intravenous administration. In some embodiments, a compound described herein (e.g., a mixed V1AR agonist-antagonist, such as Compound 1) decreases blood pH in an individual, such as after intravenous administration.

In some instances, a compound described herein (e.g., a mixed V1AR agonist-antagonist, such as Compound 1) provides an initial apparent volume of the central compartment (V_{c}) in an individual (e.g., a mammal) of at least 20 mL/kg (e.g., at least 40 mg/kg, at least 60 mg/kg, at least 80 mg/kg, at least 100 mg/kg), such as after intravenous administration. In some instances, a compound described herein (e.g., a mixed V1AR agonist-antagonist, such as Compound 1) provides an initial apparent volume of the central compartment (V_{c}) in an individual (e.g., a mammal) of at most 150 mg/kg (e.g., at most 130 mg/kg, at most 110 mg/kg, at most 90 mg/kg), such as after intravenous administration.

In some instances, a compound described herein (e.g., a mixed V1AR agonist-antagonist, such as Compound 1) provides a volume of distribution at steady state (Vₛₛ) in an individual (e.g., a mammal) of at least 120 mL/kg (e.g., at least 130 mL/kg, at least 140 mL/kg, at least 150 mL/kg), such as after intravenous administration. In some instances, a compound described herein (e.g., a mixed V1AR agonist-antagonist, such as Compound 1) provides a volume of distribution at steady state (Vₛₛ) in an individual (e.g., a mammal) of at most 200 mL/kg (e.g., at most 180 mL/kg, at most 160 mL/kg, at most 140 mL/kg), such as after intravenous administration.

In some instances, a compound described herein (e.g., a mixed V1AR agonist-antagonist, such as Compound 1) provides a maximum plasma concentration (Cₘₐₓ) per unit dose in an individual (e.g., a mammal) of at least 200 ng/mL per mg/kg (e.g., at least 300 ng/mL per mg/kg, at least 500 ng/mL per mg/kg, at least 600 ng/mL per mg/kg, at least 700 ng/mL per mg/kg), such as after subcutaneous administration. In some instances, a compound described herein (e.g., a mixed V1AR agonist-antagonist, such as Compound 1) provides a maximum plasma concentration (Cₘₐₓ) per unit dose in an individual (e.g., a mammal) of at most 1000 ng/mL per mg/kg (e.g., at most 900 ng/mL per mg/kg, at most 700 ng/mL per mg/kg, at most 500 ng/mL per mg/kg, at most 400 ng/mL per mg/kg), such as after subcutaneous administration. In some instances, a compound described herein (e.g., a mixed V1AR agonist-antagonist, such as Compound 1) provides a time of maximum plasma concentration (Tₘₐₓ) in an individual (e.g., a mammal) of at least 5 minutes (e.g., at least 10 minutes, at least 20 minutes, at least 30 minutes), such as after subcutaneous administration In some instances, a compound described herein (e.g., a mixed V1AR agonist-antagonist, such as Compound 1) provides a time of maximum plasma concentration (Tₘₐₓ) in an individual (e.g., a mammal) of at most 60 minutes (e.g., at most 50 minutes, at most 40 minutes, at most 30 minutes, at most 20 minutes), such as after subcutaneous administration.

In some instances, a compound described herein (e.g., a mixed V1AR agonist-antagonist, such as Compound 1) provides an area under the curve (AUC) per unit dose in an individual (e.g., a mammal) of at least 40,000 min-ng/mL per mg/kg (e.g., at least 45,000 min-ng/mL per mg/kg, at least 50,000 min-ng/mL per mg/kg, at least 55,000 min-ng/mL per mg/kg, at least 60,000 min-ng/mL per mg/kg), such as after subcutaneous administration. In some instances, a compound described herein (e.g., a mixed V1AR agonist-antagonist, such as Compound 1) provides an area under the curve (AUC) per unit dose in an individual (e.g., a mammal) of at most 70,000 min-ng/mL per mg/kg (e.g., 65,000 min-ng/mL per mg/kg, 60,000 min-ng/mL per mg/kg, 55,000 min-ng/mL per mg/kg, 50,000 min-ng/mL per mg/kg), such as after subcutaneous administration.

In some instances, a compound described herein (e.g., a mixed V1AR agonist-antagonist, such as Compound 1) provides an apparent total body clearance (CL/F) in an individual (e.g., a mammal) of at least 10 mL/min/kg (e.g., at least 12 mL/min/kg, at least 15 mL/min/kg, at least 18 mL/min/kg), such as after subcutaneous administration. In some instances, a compound described herein (e.g., a mixed V1AR agonist-antagonist, such as Compound 1) provides an apparent total body clearance (CL/F) in an individual (e.g., a mammal) of at most 30 mL/min/kg (e.g., at most 28 mL/min/kg, at most 25 mL/min/kg, at most 22 mL/min/kg, at most 20 mL/min/kg), such as after subcutaneous administration.

In some instances, an individual receiving a treatment described herein achieves one or more outcome measurement described herein, such as described in the Examples, while and/or subsequent to receiving treatment. In some instances, an individual receiving a treatment described herein meets one or more inclusion criteria provided in the Examples. In some instances, an individual receiving a treatment described herein meets each inclusion criteria provided in the Examples. In some instances, an individual receiving a treatment described herein fails to meet one or more exclusion criteria provided in the Examples. In some instances, an individual receiving a treatment described herein fails to meet each exclusion criteria provided in the Examples. In some embodiments, an individual receiving a treatment described herein is administered a compound described herein (e.g., Compound 1) until one or more primary and/or secondary outcome measurements, such as a primary and/or secondary outcome measurement provided in the Examples, is met. In some instances, an individual is administered a compound described herein (e.g., Compound 1) until the individual has a sCr value of 1.5 milligrams (mg)/deciliters (dL) or less on (e.g., 2 or more) consecutive days. In some embodiments, a compound, or the pharmaceutically acceptable salt thereof, described herein (e.g., Compound 1) is (e.g., subcutaneously) administered to an individual (e.g., described herein) at least until the sCr value of the individual returns to normal (e.g., baseline).

Unless stated otherwise, a measurement described herein (such as a measurement in sCr, MAP, etc.) can be measured immediately before, hours before, days before, or weeks before a compound described herein (e.g., Compound 1), or a pharmaceutically acceptable salt (e.g., an acetate salt), is administered to an individual receiving a treatment described herein.

In some embodiments, an individual receiving a treatment described herein has a reduction in sCr. In some embodiments, an individual receiving a treatment described herein has a substantial reduction in sCr. In some embodiments, an individual receiving a treatment described herein has a significant reduction in sCr. In some embodiments, an individual receiving a treatment described herein has a reduction in sCr value of about 10% or more, such as compared to a baseline measurement before treatment. In some embodiments, an individual receiving a treatment described herein has a reduction in sCr value of about 50% or less, such as compared to a baseline measurement before treatment. In some embodiments, an individual receiving a treatment described herein has a reduction in sCr value of about 10% to about 50%, such as compared to a baseline measurement before treatment. In some embodiments, an individual receiving a treatment described herein has a reduction in sCr value of about 20% to about 50%, such as compared to a baseline measurement before treatment. In some embodiments, an individual receiving a treatment described herein has a reduction in sCr value of about 30% to about 50%, such as compared to a baseline measurement before treatment. In some embodiments, an individual receiving a treatment described herein has a reduction in sCr value of about 40% to about 50%, such as compared to a baseline measurement before treatment. In some instances, the reduction in sCr value after treatment with a compound described herein (e.g., Compound 1), or the pharmaceutically acceptable salt (e.g., the acetate salt), is significantly more compared to the reduction in sCr value after treatment with other treatment options.

In some embodiments an individual receiving a treatment described herein is administered a compound described herein (e.g., Compound 1), or the pharmaceutically acceptable salt (e.g., the acetate salt), to the individual until the individual has a sCr value of 1.5 milligrams (mg)/deciliters (dL) or less on consecutive days. In some embodiments an individual receiving a treatment described herein is administered a compound described herein (e.g., Compound 1), or the pharmaceutically acceptable salt (e.g., the acetate salt), to the individual until the individual has a sCr value of 1.5 mg/dL or less on 2 or more consecutive days.

In some embodiments, the mean arterial pressure (MAP) of an individual receiving a treatment described herein increases. In some embodiments, the MAP of an individual receiving a treatment described herein increases compared to a baseline measurement before treatment. In some embodiments, the MAP of an individual receiving a treatment described herein increases (e.g., compared to a baseline measurement before treatment) by about 15 mmHg or less.

In some embodiments, the mean arterial pressure (MAP) of an individual receiving a treatment described herein decreases. In some embodiments, the MAP of an individual receiving a treatment described herein decreases compared to a baseline measurement before treatment. In some embodiments, the MAP of an individual receiving a treatment described herein decreases (e.g., compared to a baseline measurement before treatment) by about 15 mmHg or less.

In some embodiments, the MAP of an individual receiving a treatment described herein remains largely (e.g., significantly) unchanged (e.g., compared to a baseline measurement before treatment). In some embodiments, the MAP of an individual receiving a treatment described herein remains largely (e.g., significantly) unchanged compared to a baseline measurement before treatment.

Provided in some instances herein is a method of modulating mean arterial pressure (MAP) in an individual (e.g., in need thereof), the method comprising: (a) administering (e.g., intravenously or subcutaneously) an effective amount of a first compound, or a pharmaceutically acceptable salt thereof, the first compound being a vasoconstrictor (e.g., a vasopressin receptor 1A (V1AR) agonist (e.g., a selective V1AR agonist)); and (b) administering (e.g., intravenously or subcutaneously) a second compound (e.g., a V1AR antagonist (e.g., a selective V1AR antagonist) or a vasodilator), or a pharmaceutically acceptable salt thereof, wherein the second compound sufficiently blocks a (local) vasoconstrictive effect of the first compound (on the individual), thereby providing sufficient uptake of the first compound to the circulatory system and/or an internal organ (e.g., a kidney) of the individual. In some instances, such as subsequent to administration, the first compound constricts blood vessel(s) near or at the site of administration, thereby preventing sufficient uptake of the first compound to the internal organ (e.g., the kidney) of the individual. In some instances, the first compound and the second compound are concurrently administered to the individual. In some instances, administration of the second compound reduces the local vasoconstrictive effect of the first compound but not the vasoconstrictive effect of the first compound on the internal organ (e.g., kidney) of the individual. In some instances, administration of the second compound reduces the local vasoconstrictive effect of the first compound such that a therapeutically effective amount of the first compound is delivered to the internal organ (e.g., kidney) of the individual. In some instances, the individual has HRS-AKI.

In some embodiments, an individual receiving a treatment described herein has a Model for End-Stage Liver Disease (MELD) score of up to 35 before treatment, such as at the time of randomization, such as days before treatment.

In some embodiments, an individual receiving a treatment described herein has proteinuria of up to about 500 mg/dL before treatment, such as at the time of randomization, such as days before treatment.

In some embodiments, an individual receiving a treatment described herein has an oxygen flow of at least about 90% on 2 liter (L) or less before treatment, such as at the time of randomization, such as days before treatment.

In some embodiments, an individual receiving a treatment described herein has a pulse oximeter reading of at least about 90% on 2 liter (L) before treatment, such as at the time of randomization, such as days before treatment.

In some embodiments, an individual receiving a treatment described herein has a systolic blood pressure of up to 140 mmHg. In some embodiments, an individual receiving a treatment described herein has a diastolic blood pressure of up to 100 mmHg. In some embodiments, an individual receiving a treatment described herein has a systolic blood pressure of 140 mmHg or less and a diastolic blood pressure of 100 mmHg or less. In some instance the systolic blood pressure and/or diastolic blood pressure of the individual are measured before treatment, such as at the time of randomization, such as weeks before treatment.

In some embodiments, an individual receiving a treatment described herein is receiving albumin. In some embodiments, an individual receiving a treatment described herein is receiving albumin and has had appropriate diuretic withdrawal before treatment. In some embodiments, an individual receiving a treatment described herein is receiving albumin and has had appropriate diuretic withdrawal days before treatment. In some embodiments, an individual receiving a treatment described herein is receiving albumin and has had appropriate diuretic withdrawal at least 48 hrs before treatment.

In some embodiments, an individual receiving a treatment described herein has a lack in sustained renal function improvement. In some embodiments, an individual receiving a treatment described herein has a lack in sustained renal function improvement after diuretic withdrawal. In some embodiments, an individual receiving a treatment described herein has a lack in sustained renal function improvement after plasma volume expansion with albumin. In some embodiments, an individual receiving a treatment described herein has a lack in sustained renal function improvement after diuretic withdrawal and plasma volume expansion with albumin.

In some instances, an individual receiving a treatment described herein is in need of the treatment described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Examples

### Example 1: Tolerability, Pharmacokinetic (PK), and Pharmacodynamic (PD) Profiles for Mixed V1AR Agonist-Antagonists in Healthy Humans and Rats after Intravenous (IV) and Subcutaneous (SC) Administration

In summary, systemic exposure of a compound described herein (e.g., a mixed V1AR agonist-antagonist, such as Compound 1) was measured after a composition comprising the compound was administered to healthy rats and humans by IV infusion and SC (bolus) injection. Additionally, systemic effects, such as modulation of mean arterial pressure (MAP), were measured after the composition was administered to healthy rats and humans by IV infusion and SC (bolus) injection.

While the systemic effects observed after IV infusion and SC (bolus) injection were comparable, more adverse events were measured in individuals receiving SC (bolus) injection of the composition. Generally, the compound was well-tolerated by individuals receiving the composition by IV infusion.

A substantial amount of a metabolite (M1—a full (V1a) agonist) was measured in healthy humans and rats after the composition was administered by subcutaneous (bolus) injection. Specifically, in both healthy humans and rats, about 80-90% of the metabolite M1 was measured after subcutaneous (bolus) injection of the composition. In contrast, little to no M1 was measured after intravenous infusion of the composition. As discussed herein, full vasopressin receptor agonists described herein (e.g., terlipressin) are known to cause (serious) adverse events when administered subcutaneously. As such, the substantial metabolic conversion of the compound to a full agonist (M1) after SC (bolus) injection provides an explanation for the differences between the tolerability profile of the composition in healthy individuals after IV infusion and (SC) bolus injection. Additional studies described below demonstrate ways to reduce full agonist (M1) formation after SC administration of a composition comprising a mixed V1AR agonist-antagonist described herein, such as Compound 1.

### Trial Design Summary

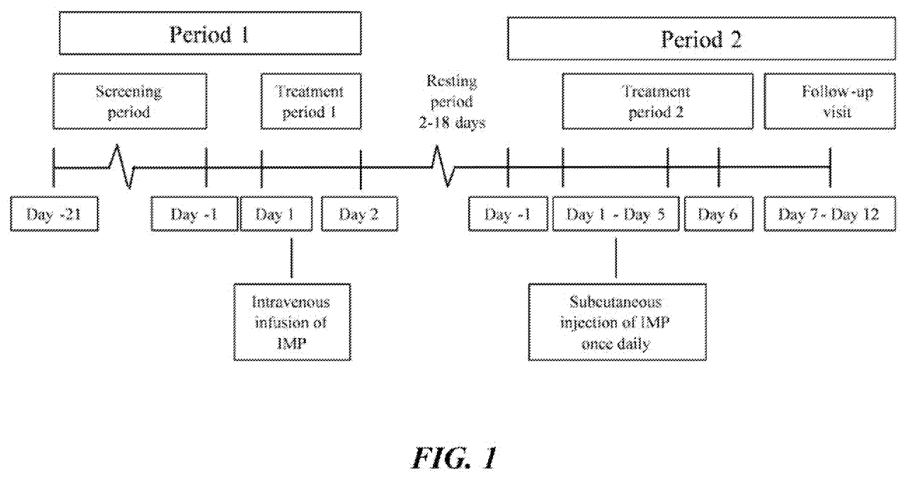

A double-blind, placebo controlled, within dose-group randomized trial investigating the safety, tolerability, pharmacokinetics, and pharmacodynamics of Compound 1 administered as an intravenous infusion and multiple subcutaneous injections in healthy men and women. The trial comprised two treatment periods, treatment Period 1 and treatment Period 2 (see FIG. 1).

The study was conducted in two periods where the treatment in Period 1 was administered as an intravenous infusion, and the treatment in Period 2 was administered as subcutaneous injections. The trial comprised 5 ascending dose panels, each dose panel comprising 8 healthy subjects (6 active, 2 placebo). A new cohort of subjects was used for each dose panel.

Treatment period 1: The subjects received a 6-hour intravenous infusion of Compound 1 or placebo. At each dose level, the first two subjects to be infused were randomized to receive either Compound 1 or placebo, one of each, with a minimum observation period of 24 hours. If no safety concerns arose, as judged by the Investigator, the remaining subjects in that dose group were randomized and treated according to the randomization and protocol in a staggered dosing manner. The dose escalation included 0.1, 0.3, 0.45, 0.6, and 0.9 milligrams (mg) of Compound 1.

Treatment period 2: After a resting period of 2-18 days each subject received a once daily subcutaneous dose for 5 days of the same treatment as in Period 1. The dose was selected to prevent the exposure to exceed the intravenous exposure. The dose escalation was stopped after the 0.3 mg dose panel due to the risk of exceeding the maximum tolerated dose with higher doses. For each subject the treatment periods were 2 plus 6 days, and the total duration of the study from the screening visit to the follow-up visit after treatment did not exceed 8 weeks. A SRC evaluated the safety and tolerability of the compound after the completion of all subjects in the dose group.

All subjects were allocated to active treatment or placebo in a double-blind setting, i.e. the active or placebo treatment was not revealed to the subjects or the personnel involved in the study. The randomization and blinding were performed to reduce any bias concerning reporting of e.g. AEs.

A single intravenous infusion over 6 hours and a once daily subcutaneous dose for 5 days were selected as routes of administration and duration of treatment (e.g., to investigate the full tentative dose range of Compound 1). The continuation of the same subjects from intravenous to subcutaneous administration was selected to get an as reliable as possible estimate of the bioavailability (F) in the small groups outlined.

The first two subjects to be infused at each dose level was one receiving active Compound 1, and one receiving placebo with a minimum observation period of 24 hours in order to decrease the risks related to exposure to a substance in early clinical development. The sequential dose-escalation design with safety assessments of the preceding dose level was chosen in consideration of the safety of the study subjects.

The originally planned escalating doses were 0.1, 0.3, 1, 3, 6, and 10 mg, respectively. In a preceding study, administration of 0.3 mg as a 6 hour intravenous infusion resulted in Cₘₐₓ concentrations of up to about 6 ng/mL. Based on data from the preceding study, the subcutaneous (s.c.) doses planned (0.1, 0.3, 1, 3, 6, and 10 mg) were estimated to reach, but not exceed, the Cₘₐₓ levels obtained with the corresponding intravenous infusions. Administration of 0.3 mg as a s.c. injection resulted in Cₘₐₓ concentration of up to about 2.5 ng/mL. The doses and/or infusion rates could be adjusted based on recommendations from the SRC.

Injection site reactions were assessed by the Investigator in treatment Period 2 only, immediately after, 0.5, 4, and 24 hours after subcutaneous administration of the study drug on Days 1 to 5. The injection site reactions to be assessed were erythema, pain, pruritus, oedema, bruising, and pallor, each to be assessed as none, mild, moderate, or severe. Injection site reactions other than those described above or persisting longer than 24 hours were classified as adverse events (AEs).

In Period 1 blood samples for safety laboratory evaluations of clinical chemistry parameters were collected at screening, on Day −1, pre-dose and 1, 2, 4, 6, 8, 12, and 24 hours after start of intravenous administration of Compound 1. Blood samples for hematology, and hemostasis parameters were collected at screening, on Day −1, and 6, 12, and 24 hours after start of intravenous administration of Compound 1.

For the subcutaneous administration in Period 2, samples for clinical chemistry parameters were collected on Day −1, pre-dose, and at 2, and 6 hours after the administration of Compound 1, and at follow-up. Blood samples for hematology and hemostasis parameters were collected on Day −1, and at 2, and 4 hours after the administration on Day 1, pre-dose, and 3 hours after the administration on Days 2-4, pre-dose, and at 2, and 6 hours after the administration on Day 5 and at follow-up. The actual sampling times were recorded.

Urine samples for safety laboratory evaluation of urinalysis parameters were in Period 1 collected at screening, on Day −1, pre-dose, and at the collecting periods 0-4 hours, 4-8 hours, 8-12 hours, and 12-24 hours after the start of the intravenous administration of Compound 1. For the subcutaneous administration in Period 2, urinalysis samples were collected on Day −1, pre-dose, 2 and 6 hours after the administration of Compound 1 on Days 1 to 5, and at follow-up. Urinalysis was performed locally by means of a dip-stick test. In case any result of the dipstick was abnormal, a new urine test was performed. In case any result of the dipstick was abnormal and clinically significant, a new urine test was to be performed. If the abnormal result was confirmed, further examination could be initiated at the discretion of the Investigator. Clinically significant abnormal findings were reported as AEs. The metabolite pattern of Compound 1 in plasma and urine were measured.

A similar study in healthy rats was used to evaluate the differences between metabolite formation of Compound 1 after IV bolus and SC (bolus) injection.

### Eligibility

Individuals of the study meet the inclusion criteria provided in Table 1.

TABLE 1
Inclusion criteria

Signed written Informed Consent
Healthy men and women, 18-45 years of age
A body mass index (BMI) of 18.5-32.0 kg/m² (both inclusive)
Negative serology for human immunodeficiency virus (HIV)-1, HIV-2, hepatitis B (surface
antigen), hepatitis C antibody
Healthy according to medical history, physical examination, 12-lead ECG, blood pressure,
and laboratory profile of blood and urine
Systolic and diastolic blood pressure below the upper limit of the normal range i.e. <140/90
Negative pregnancy test at Screening and on Day -1 in both periods
Agreed to use an adequate method of contraception during the study until the follow-up visit,
if not abstinent. Adequate methods of contraception included condom, diaphragm, coil
(intrauterine device), surgical sterilization, vasectomy, oral contraceptive pill, depot
progesterone injections
Negative urine drug screen and breath alcohol test at Screening and on Day -1 in both periods
Non-smoker or light smoker (average of <7 cigarettes per week) or had stopped smoking or
become light smoker >6 months prior to study start with no history of long-term, heavy
smoking (>10 pack-years)

Individuals having any one of the exclusion criteria provided in Table 2 were excluded from the study.

TABLE 2
Exclusion criteria

Presence or a history of clinically significant diseases of the renal, hepatic, gastrointestinal,
cardiovascular, musculoskeletal, or gynecological systems, or presence or history of
clinically significant psychiatric, immunological, endocrine or metabolic diseases
Malignant disease within the last 5 years except for adequately managed basal cell carcinoma
and squamous cell carcinoma of the skin
Women being pregnant or breastfeeding
Presence or history of severe allergy or anaphylactic reactions
Hypersensitivity towards any component of Compound 1 formulation
Any clinically relevant abnormal findings in medical history, physical examination, vital
signs, 12-lead ECG, clinical chemistry, hematology, hemostasis, urinalysis at screening or
Day -1, which in the opinion of the investigator(s), could put the subject at risk because of
his/her participation in the study or provide difficulties in interpreting the study data
Intake of prescribed medication, over-the-counter medication, or herbal medicines within 2
weeks or 5 half-lives of the drug, whichever was longer prior to the dose of Compound 1.
Paracetamol, cromoglycate and oral contraceptives according to label were allowed
Intake of an IMP within the last 12 weeks preceding screening or longer if judged by the
investigator to possibly influence the outcome of the current study
Blood donation or major blood loss (≥500 mL) within the last 12 weeks preceding the first
day of dosing
History within the last two years, or current abuse of alcohol or drugs
Previous participation in this study
Mental incapacity or language barrier precluding adequate understanding or co-operation
Considered by the investigator to be unsuitable to participate in the study for any other reason

Dose escalation was to be terminated if one or more of the pre-defined criteria provided in Table 3, as confirmed by a second measurement, were met in at least two subjects on active treatment in a dose panel.

TABLE 3
Exclusion criteria

Systolic blood pressure increased by 50% or more from the baseline value
Systolic blood pressure 180 mmHg or higher
Diastolic blood pressure 105 mmHg or higher
Mean arterial pressure (MAP) 130 mmHg or higher
Cardiac output reduced by 25% or more as estimated by echocardiography
Signs or symptoms of hypertensive crises

### Subjects

85 subjects were screened in this trial, 64 of whom were randomized and dosed. In Period 1, intravenous (i.v.) administration, there were 5 dose groups, 0.1, 0.3, 0.45, 0.6, and 0.9 mg, and in Period 2, subcutaneous (s.c.) administration, there were 2 dose groups, 0.1 and 0.3 mg. The 0.1 mg and 0.3 dose groups (intravenous infusion followed by subcutaneous administration) initially comprised 8 subjects, 6 of whom received active treatment and 2 of whom received placebo. All subjects in the 0.1 mg dose group received both i.v. and s.c. administration, while in the 0.3 mg dose group only 3 of the 6 subjects being infused continued to s.c. administration. The 0.45, 0.6, and 0.9 mg dose groups (intravenous administration) each comprised 16 subjects, 12 of whom received active treatment and 4 of whom received placebo. All subjects on active treatment in the 0.1, 0.45, 0.6, and 0.9 mg dose groups completed the trial, while 1 subject on active treatment in the 0.3 mg dose group discontinued the trial due to an AE after the 1^{st} subcutaneous administration (1 event of elevated Troponin I levels). Since it was decided to terminate s.c. administration after 3 subjects in the 0.3 mg dose group, the remaining 3 subjects are defined as completed. All subjects receiving placebo completed the trial.

### Treatments

In Period 1, subjects received a single 6-hour intravenous infusion of either Compound 1 or placebo at a constant rate. The intravenous dose to the first dose panel was the lowest dose giving a clear signal of pharmacological activity in the preceding trial. The planned and actual doses of Compound 1 during intravenous infusion are provided in Table 4. The administration was performed by using a piston-driven syringe auto-infusion pump. The administered volume was recorded for each subject.

TABLE 4

| | Planned dose | Actual dose |
|---|---|---|
| Group | (milligrams (mg)) | (milligrams (mg)) |
| Group 1 | 0.1 | 0.1 |
| Group 2 | 0.3 | 0.3 |
| Group 3 | 1* | 0.45 |
| Group 4 | 3* | 0.6 |
| Group 5 | 6* | 0.9 |
| Group 6 | 10* | — |

*Dose escalation was changed after the 0.3 mg dose panel for both female and male subjects to an escalation in smaller increments i.e. 0.45, 0.6, and 0.9 mg due to an anticipated risk of exceeding the maximum tolerated dose at higher doses.

In Period 2, subjects received a once-daily subcutaneous injection for 5 days of the same treatment as in Period 1 at different locations on the abdomen (Table 5). The subcutaneous doses were selected based on data from a previous trial.

TABLE 5

| | Planned dose | Actual dose |
|---|---|---|
| Group | (milligrams (mg)) | (milligrams (mg)) |
| Group 1 | 0.1 | 0.1 |
| Group 2 | 0.3 | 0.3 |
| Group 3 | 1* | — |
| Group 4 | 3* | — |
| Group 5 | 6* | — |
| Group 6 | 10* | — |

*Dose escalation was stopped after the 0.3 mg dose panel for both female and male subjects due to the possibility to exceed the maximum tolerated dose with higher doses.

### Formulation

In some instances, Compound 1 is formulated as an aseptically manufactured aqueous solution of Compound 1, 1 mg/mL to about 10 mg/mL, in 10 mM acetate buffer pH 4.5 with mannitol (e.g., for isotonicity). In some instances, the formulation comprising Compound 1 is filled into glass vials (1.5 mL withdrawable volume) and sealed with rubber stoppers and plastic caps. In some instances, the formulation comprising Compound 1 is diluted to the appropriate concentrations with 0.9% sodium chloride injection prior to administration.

In some instances, Compound 1 is formulated in an investigational medicinal product (IMP) as illustrated in Table 6. In some instances, Compound 1 was diluted with 5% Dextrose to the desired concentrations, and placebo was diluted to the same extent for each dose group.

TABLE 6

| | |
|---|---|
| IMP | Placebo |

| Compound 1 | 10 | mg/mL | — |
|---|---|---|---|
| Sodium acetate buffer | 10 mM, pH 4.5 | | 10 mM, pH 4.5 |
| Mannitol | 44.3 | mg/mL | 47.2 mg/mL |

### Objectives and Endpoints

In some instances, an objective of the study was to characterize the differences between intravenous and subcutaneous dosing. In a previous single-blind study, differences in adverse events (AEs) and pharmacodynamic cardiovascular changes were observed after subcutaneous compared with intravenous administration despite administration of the same dose and similar exposure.

In some instances, an objective of the study was to obtain general data for the safety, tolerability, pharmacokinetics, and pharmacodynamics of Compound 1 in healthy subjects.

In some instances, an objective of the study was to determine safety and tolerability of single doses of Compound 1 administered as a continuous intravenous infusion.

In some instances, an objective of the study was to determine safety and tolerability of multiple doses of Compound 1 administered as daily subcutaneous injections.

In some instances, an objective of the study was to determine single-dose intravenous and multiple-dose subcutaneous pharmacokinetics of Compound 1.

In some instances, an objective of the study was to investigate metabolite pattern of Compound 1 in plasma and urine.

In some instances, an objective of the study was to investigate the relationship between pharmacokinetics and pharmacodynamics of Compound 1 in healthy subjects.

In some instances, endpoints of the study were: vital signs (e.g., supine blood pressure, pulse, and body temperature), electrocardiogram (ECG) (e.g., intervals, rhythm, and morphology), cardiac function (e.g., cardiac output using echocardiography), peripheral blood flow/tissue perfusion (e.g., by skin color), venous blood gases (e.g., lactate), urinary output, clinical chemistry, hematology, hemostasis, and urinalysis, adverse events (AEs) (e.g., type, frequency, and intensity), pharmacokinetics (e.g., AUC, AUCₜ, AUC_{τ}, % Extrap AUC, Cₘₐₓ, tₘₐₓ, CL, V_{z}, t_{1/2}, MRT, Vₛₛ, F, Ae, and CLR), and metabolite pattern in plasma and urine.

### Results

Generally, Compound 1 was systemically delivered and produced systemic effects when administered to healthy individuals by IV infusion and SC (bolus) injection. However, while little to no formation of M1 (e.g., about 0-15%) occurred after Compound 1 was administered to healthy humans by intravenous infusion (FIG. 44, panel A), the concentration of Compound 1 and M1 were about equimolar in healthy humans after Compound 1 was administered by subcutaneous (bolus) injection (FIG. 44, panel B). Specifically, both human and rat data indicate that about 80-90% of Compound 1 was converted to M1 (e.g., in the subcutaneous space) after SC (bolus) injection. These results demonstrate that substantial amounts of M1 (a full (V1) agonist) form after SC (bolus) injection but not IV infusion, providing an explanation of why healthy humans administered Compound 1 via SC (bolus) injection experienced more adverse events than healthy humans who received Compound 1 by IV infusion.

### Tolerability and Summary of Adverse Events (AEs):

94 treatment emergent adverse events (TEAEs) occurred in 35 of the 48 subjects on active treatment, and 8 TEAEs occurred in 5 of the 16 subjects on placebo after the intravenous infusion in Period 1. 87 TEAEs occurred in the 9 subjects on active treatment, and 4 TEAEs occurred in 3 of the 4 subjects on placebo after the subcutaneous administration in Period 2. In Period 1, 87 of the TEAEs reported by 34 subjects on active treatment, and 6 of the TEAEs reported by 3 subjects on placebo were regarded as adverse drug reactions (ADRs) (e.g., assessed as reasonably possibly related to treatment). One AE reported by 1 subject in the 0.1 mg dose group was judged as severe. No serious AEs occurred, and neither did any AE lead to death or discontinuation of the trial. In Period 2, all 87 of the TEAEs reported by 9 subjects on active treatment, and all 4 TEAEs reported by 3 subjects on placebo were regarded as ADRs. 3 AEs reported by 3 subjects in the 0.1 mg dose group, and 2 AEs reported by 1 subject in the 0.3 mg group were judged as severe, and 1 AE reported by 1 subject in the 0.3 mg group was judged as serious and led to discontinuation of the trial for this subject. No AE led to death. The majority of TEAEs in females as well as males, and during intravenous as well as subcutaneous administration, were of mild or moderate intensity. Six events were reported as being of severe intensity; one event of bradycardia reported after intravenous infusion of 0.1 mg, 3 events of abdominal pain reported after subcutaneous administration of 0.1 mg, and 1 event of abdominal pain and 1 event of back pain, both reported by the same subject after subcutaneous administration of 0.3 mg. One event of elevated troponin I levels reported after subcutaneous administration of 0.3 mg were reported as serious.

### Pharmacokinetics:

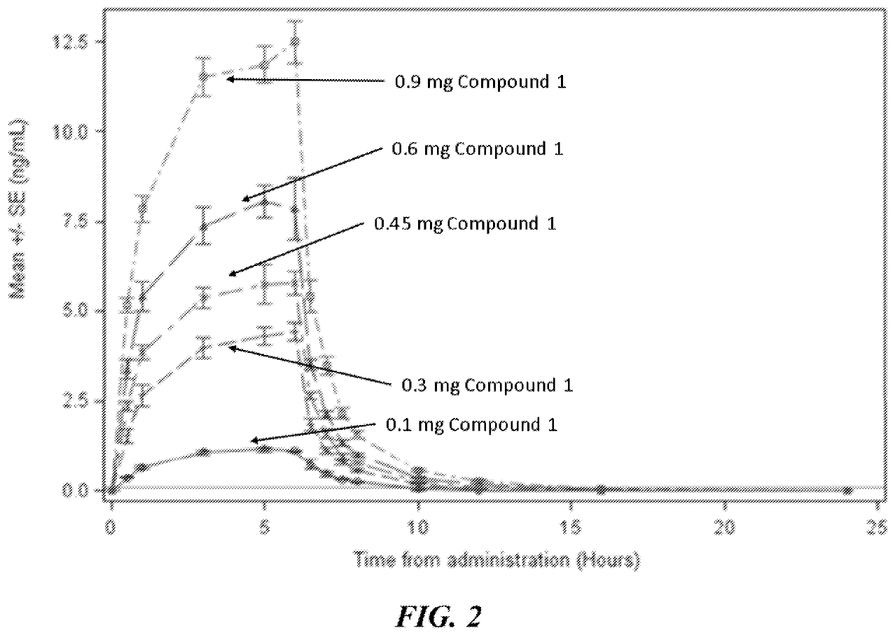

i.v.: The median time for reaching maximal serum concentration after the 6-hour intravenous infusion was between 5 and 6 hours in all dose groups (see FIG. 2). AUC and Cₘₐₓ increased with increasing doses, and analysis of dose proportionality of AUC and Cₘₐₓ indicated proportionality for both parameters over the dose range 0.1-0.9 mg. The harmonic mean terminal half-life was slightly longer in the 0.6 and 0.9 mg dose groups, about 1.5 and 1.7 hours, compared with the other dose groups, which had similar mean t₁₂ of about 1.2-1.3 hours. The other dose independent pharmacokinetic parameters were comparable between the five doses.

The pharmacokinetic results for Compound 1 i.v. infusion is shown in Table 7.

TABLE 7

| | | | | |
|---|---|---|---|---|
| 0.1 mg | 0.3 mg | 0.45 mg | 0.6 mg | 0.9 mg |
| (N = 5) | (N = 6) | (N = 12) | (N = 11) | (N = 12) |

AUC (h*ng/mL)

| | | | | | | | | | | |
|---|---|---|---|---|---|---|---|---|---|---|
| Mean (SD) | 7.01 | (1.00) | 24.9 | (3.62) | 34.5 | (5.05) | 47.5 | (9.60) | 72.5 | (10.7) |
| Geometric mean (CV %) | 6.96 | (14) | 24.6 | (14) | 34.1 | (15) | 46.5 | (21) | 71.8 | (15) |
| Min; Max | 6.16; | 8.73 | 20.4; | 30.3 | 25.7; | 43.0 | 29.1; | 66.8 | 55.8; | 95.3 |

Cₘₐₓ (ng/mL)

| | | | | | | | | | | |
|---|---|---|---|---|---|---|---|---|---|---|
| Mean (SD) | 1.19 | (0.12) | 4.49 | (0.66) | 6.26 | (0.90) | 8.45 | (1.52) | 12.8 | (2.02) |
| Geometric mean (CV %) | 1.18 | (10) | 4.45 | (15) | 6.20 | (15) | 8.32 | (18) | 12.7 | (16) |
| Min; Max | 1.09; | 1.39 | 3.55; | 5.30 | 4.76; | 7.83 | 5.66; | 11.7 | 9.26; | 16.5 |

tₘₐₓ (h)

| Mean (SD) | 5.4 | (0.5) | 5.2 | (1.2) | 5.0 | (1.0) | 5.4 | (0.9) | 5.3 | (1.2) |
|---|---|---|---|---|---|---|---|---|---|---|
| Median | 5.0 | | 5.5 | | 5.0 | | 6.0 | | 6.0 | |
| Min; Max | 5.0; | 6.0 | 3.0; | 6.0 | 3.0; | 6.0 | 3.0; | 6.0 | 3.0; | 6.0 |

T_{1/2} (h)

| Mean (SD) | 1.3 | (0.4) | 1.3 | (0.3) | 1.4 | (0.3) | 1.5 | (0.2) | 2.0 | (0.7) |
|---|---|---|---|---|---|---|---|---|---|---|
| Harmonic Mean | 1.2 | | 1.2 | | 1.3 | | 1.5 | | 1.7 | |
| Min; Max | 0.9; | 1.7 | 1.0; | 1.7 | 0.8; | 1.7 | 1.3; | 1.9 | 1.1; | 3.2 |

Tot. CL (L/h)

| | | | | | | | | | | |
|---|---|---|---|---|---|---|---|---|---|---|
| Mean (SD) | 14.5 | (1.8) | 12.3 | (1.7) | 13.3 | (2.1) | 13.2 | (3.0) | 12.7 | (1.8) |
| Geometric mean (CV %) | 14.4 | (14) | 12.2 | (15) | 13.2 | (15) | 12.9 | (22) | 12.5 | (15) |
| Min; Max | 11.5; | 16.2 | 9.9; | 14.7 | 10.5; | 17.5 | 9.0; | 20.6 | 9.4; | 16.1 |

Renal CL (L/h)

| | | | | | | | | | | |
|---|---|---|---|---|---|---|---|---|---|---|
| Mean (SD) | 0.87 | (0.07) | 0.66 | (0.09) | 0.89 | (0.28) | 0.95 | (0.23) | 0.95 | (0.39) |
| Geometric mean (CV %) | 0.87 | (9) | 0.66 | (15) | 0.84 | (41) | 0.93 | (26) | 0.89 | (39) |
| Min; Max | 0.78; | 0.92 | 0.52; | 0.76 | 0.36; | 1.23 | 0.56; | 1.31 | 0.49; | 1.92 |

V_{SS} (L)

| | | | | | | | | | | |
|---|---|---|---|---|---|---|---|---|---|---|
| Mean (SD) | 21.8 | (2.2) | 15.0 | (3.9) | 15.5 | (3.2) | 16.3 | (6.3) | 15.5 | (2.4) |
| Geometric mean (CV %) | 21.7 | (10) | 14.5 | (30) | 15.2 | (21) | 15.4 | (33) | 15.3 | (15) |
| Min; Max | 19.6; | 25.0 | 9.0; | 19.2 | 10.5; | 22.3 | 10.7; | 32.3 | 12.7; | 19.3 |

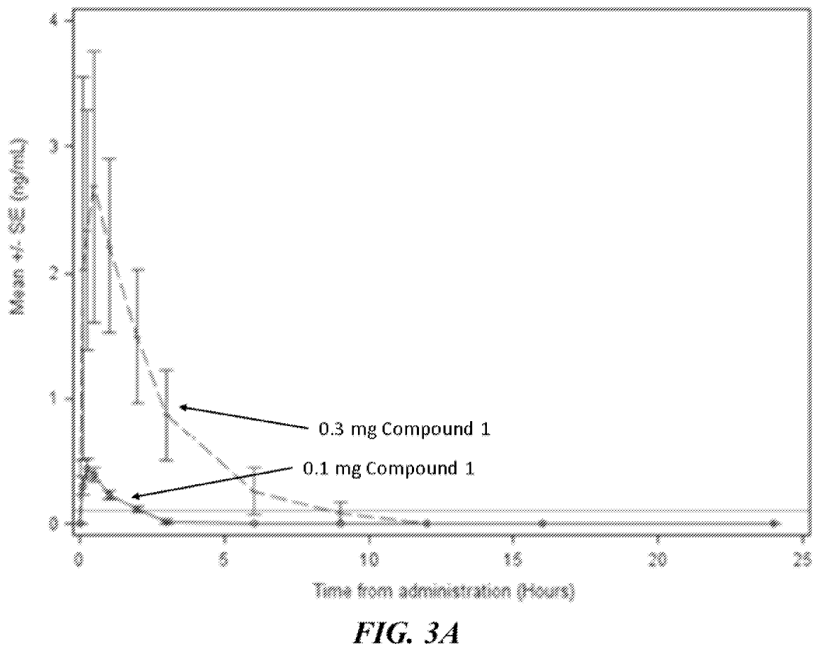

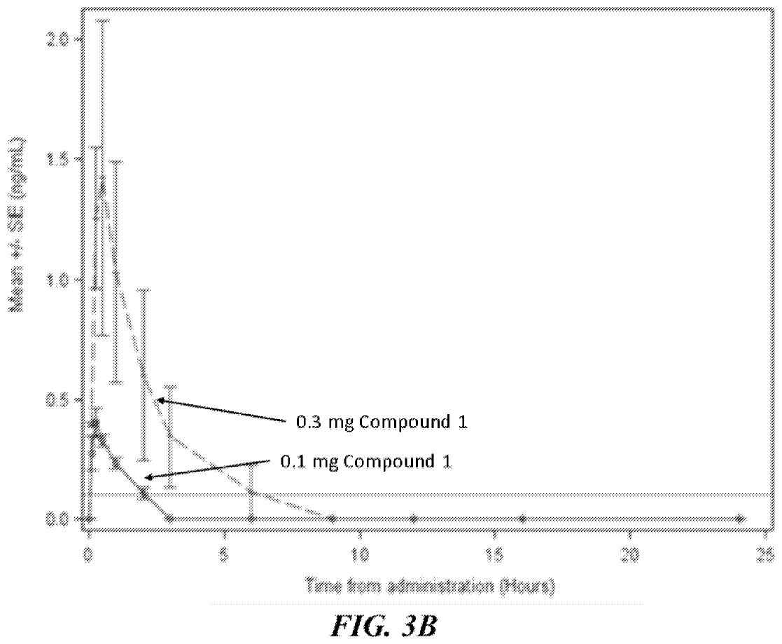

s.c.: The AUC and Cₘₐₓ increased with increasing dose after both the 1 st and 5th administration (see FIG. 3A and FIG. 3B), with slightly longer terminal half-lives in the 0.3 mg dose group (See Table 8). There was no indication of accumulation of Compound 1 after repeated administrations in any of the dose groups (See Table 8). After the 1st and 5th subcutaneous administrations the median tₘₐₓ was approx. 0.3-0.4 hours in both the 0.1 and 0.3 mg dose groups (See Table 8). The absolute subcutaneous bioavailability of Compound 1 was estimated from the intravenous data in Period 1 and the repeated subcutaneous administration in Period 2. Bioavailability of Compound 1 after repeated subcutaneous injections was estimated to 1800. The pharmacokinetic results for Compound 1 s.c. injection is shown in Table 8. Analysis of the dose proportionality of AUC and Cₘₐₓ after the 1st and 5th administered subcutaneous doses indicated possible dose proportionality for Cₘₐₓ after the 5th dose only, while AUC after both the 1 st and 5th administration and Cₘₐₓ after the 1 st administration were indicated to be greater and higher than proportional.

TABLE 8

| 1^{st} administration | | 5^{th} administration | |
|---|---|---|---|
| 0.1 mg | 0.3 mg | 0.1 mg | 0.3 mg |

AUC (h*ng/mL)

| n | 3 | | 3 | | 2 | | 2 | |
|---|---|---|---|---|---|---|---|---|
| Mean (SD) | 0.88 | (0.19) | 7.90 | (5.01) | 0.70 | (0.02) | 3.36 | (2.69) |
| Geometric mean (CV %) | 0.87 | (22) | 6.40 | (109) | 0.70 | (3) | 2.77 | (113) |
| Min; Max | 0.73; | 1.10 | 2.33; | 12.0 | 0.69; | 0.72 | 1.46; | 5.26 |

Cₘₐₓ (ng/mL)

| n | 6 | | 3 | | 6 | | 2 | |
|---|---|---|---|---|---|---|---|---|
| Mean (SD) | 0.46 | (0.17) | 2.99 | (2.03) | 0.43 | (0.14) | 1.52 | (0.79) |
| Geometric mean (CV %) | 0.43 | (41) | 2.45 | (98) | 0.42 | (28) | 1.42 | (58) |
| Min; Max | 0.24; | 0.72 | 1.01; | 5.07 | 0.32; | 0.69 | 0.97; | 2.08 |

tₘₐₓ (h)

| n | 6 | | 3 | | 6 | | 2 | |
|---|---|---|---|---|---|---|---|---|
| Mean (SD) | 0.3 | (0.1) | 0.4 | (0.5) | 0.3 | (0.1) | 0.4 | (0.2) |
| Median | 0.3 | | 0.3 | | 0.3 | | 0.4 | |
| Min; Max | 0.3; | 0.5 | 0.1; | 1.0 | 0.1; | 0.5 | 0.3; | 0.5 |

t_{1/2} (h)

| n | 3 | | 3 | | 2 | | 2 | |
|---|---|---|---|---|---|---|---|---|
| Mean (SD) | 1.1 | (0.2) | 1.6 | (0.7) | 1.0 | (0.3) | 1.4 | (0.6) |
| Harmonic Mean | 1.1 | | 1.4 | | 1.0 | | 1.3 | |
| Min; Max | 1.0; | 1.4 | 1.1; | 2.4 | 0.8; | 1.2 | 1.0; | 1.9 |

### Pharmacodynamics:

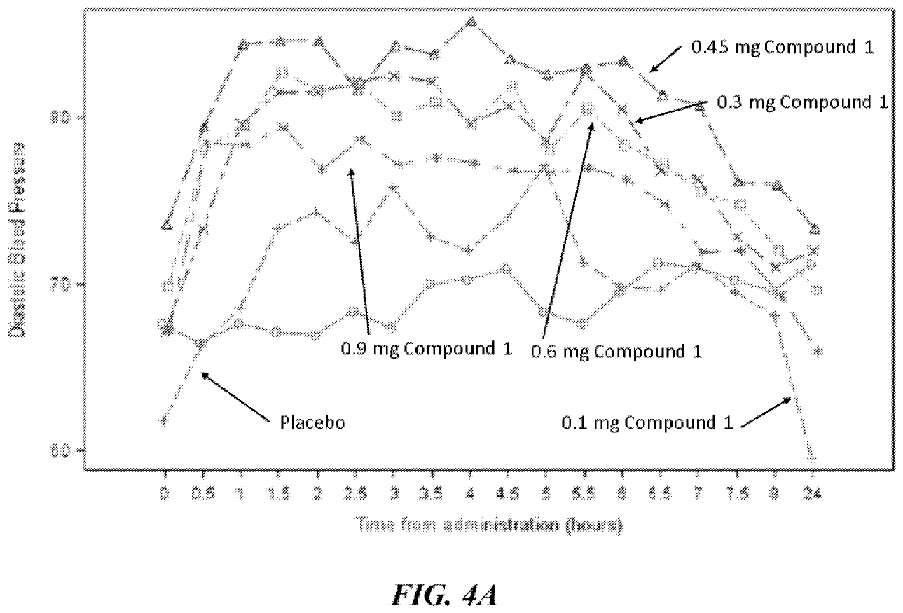

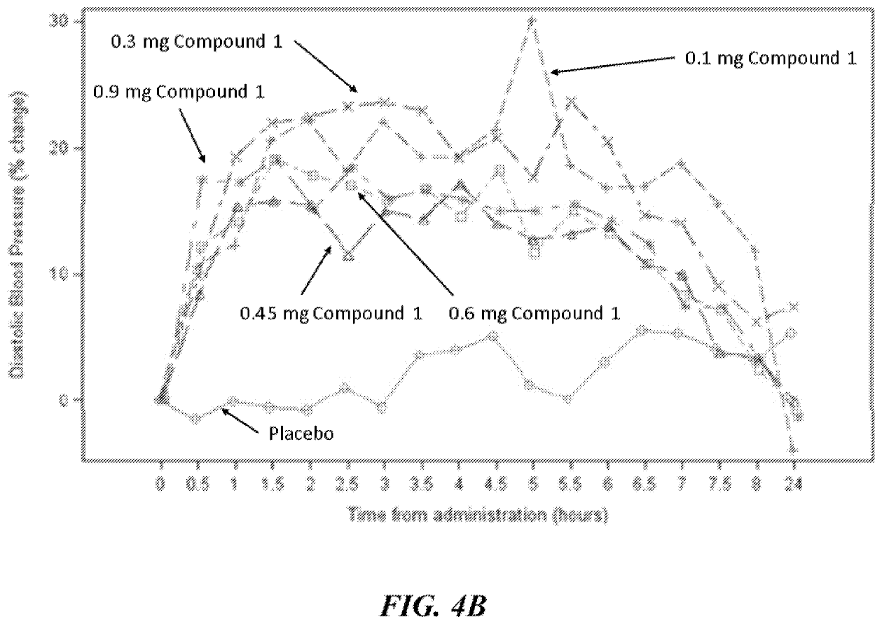

Diastolic blood pressure (i.v.): The diastolic blood pressure increased during the intravenous infusion in response to all five doses of Compound 1 (FIG. 4A and FIG. 4B) to reach a plateau after about 2 hours. The absolute as well as relative increases were comparable between all doses, the maximal mean increases being 15 (25%), 16 (24%), 12 (17%), 13 (19%), and 12 (19%) mmHg for the 0.1, 0.3, 0.45, 0.6, and 0.9 mg doses, respectively, with great inter-individual variation. Subsequent to the end of infusion the diastolic blood pressure returned to close to baseline values within about two hours.

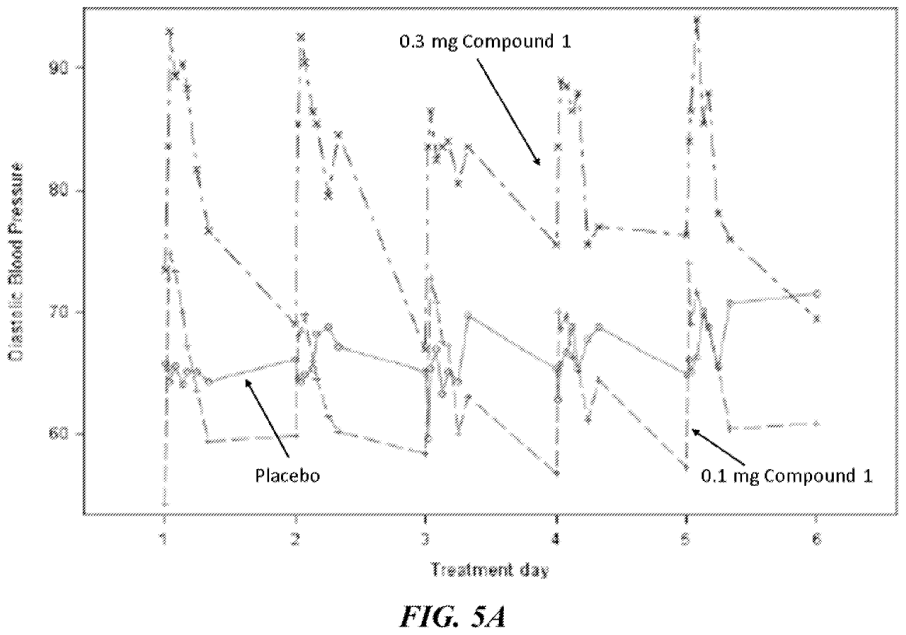

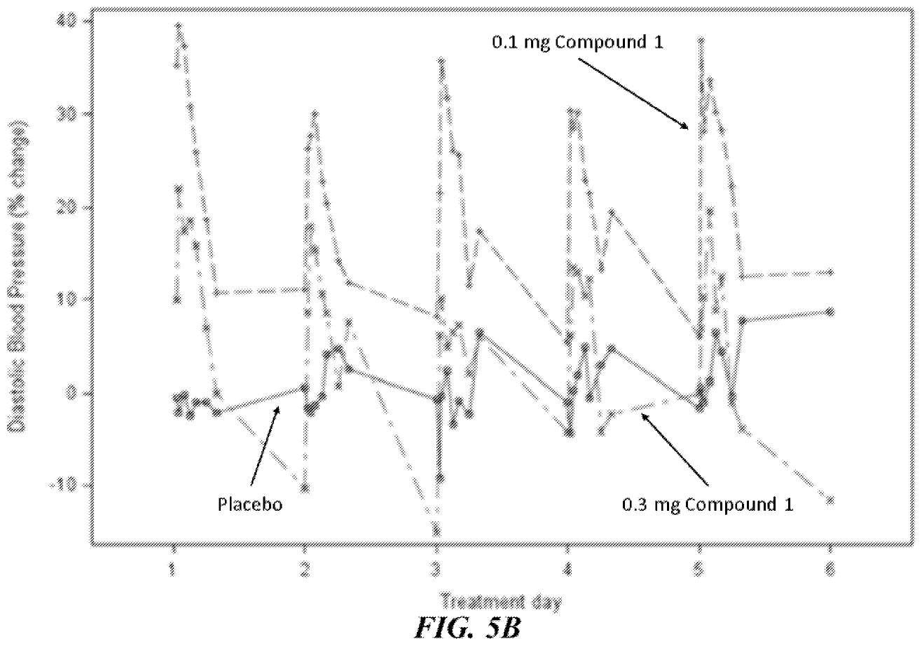

Diastolic blood pressure (s.c.): After subcutaneous administration there was a reversible increase in diastolic blood pressure in both the 0.1 and the 0.3 mg dose groups, similar for all administrations (FIG. 5A and FIG. 5B). The maximal mean increases over the 5 days of administration were 16-21 (30-40%) and 8-16 (10-22%) mmHg for the 0.1 and 0.3 mg doses, respectively (FIG. 5A and FIG. 5B), however with large inter-individual variations. The mean absolute levels and mean changes after each of the 5 administrations were similar, showing no signs of cumulative effect or sensitization/desensitization of this pharmacodynamic effect.

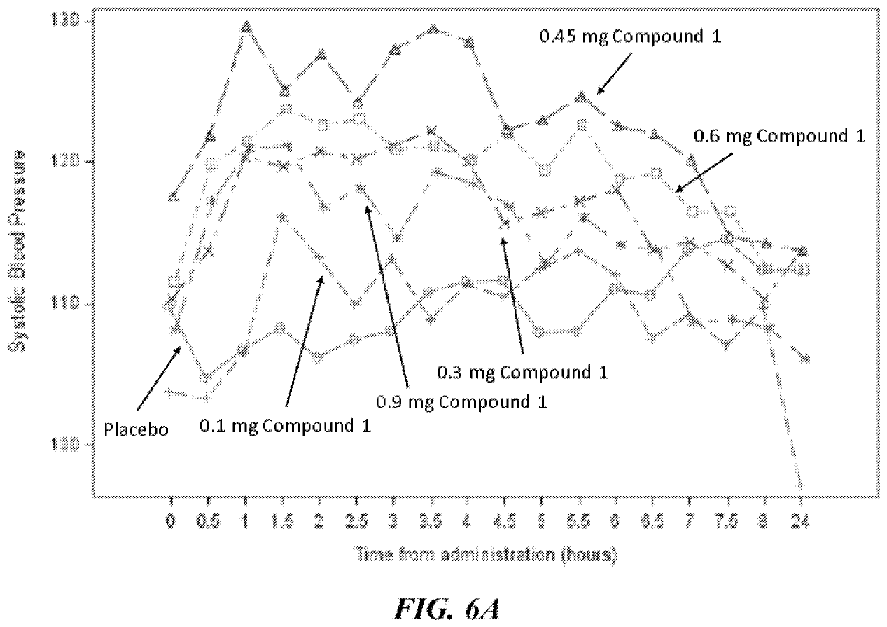

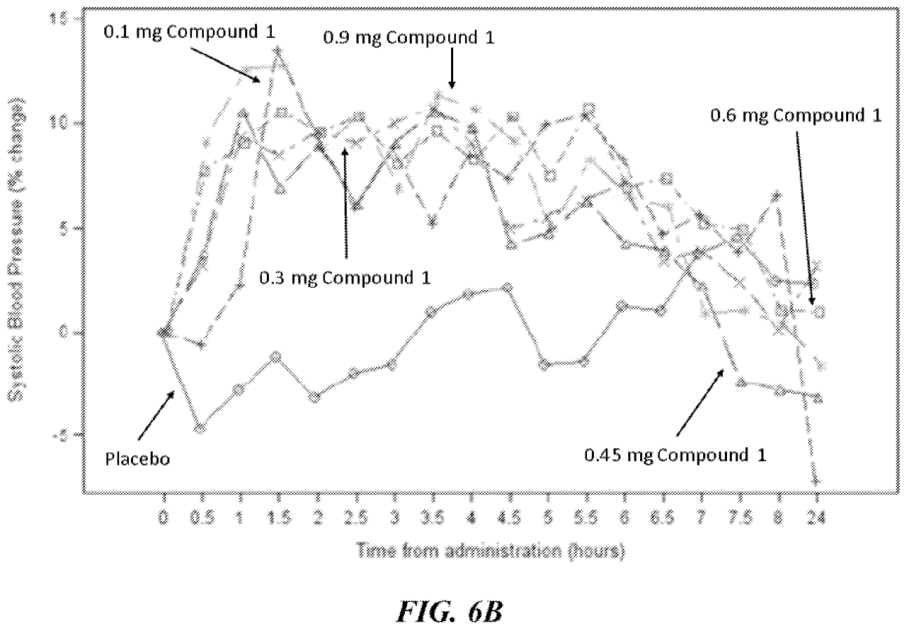

Systolic blood pressure (i.v.): The effect of Compound 1 on the systolic blood pressure was less pronounced in absolute as well as relative terms compared with the diastolic blood pressure, the maximal mean increases after intravenous infusion being 13 (13%), 12 (11%), 12 (11%), 12 (11%), and 13 (13%) mmHg for the 0.1, 0.3, 0.45, 0.6, and 0.9 mg doses, respectively (FIG. 6A and FIG. 6B), the increases being comparable for all doses.

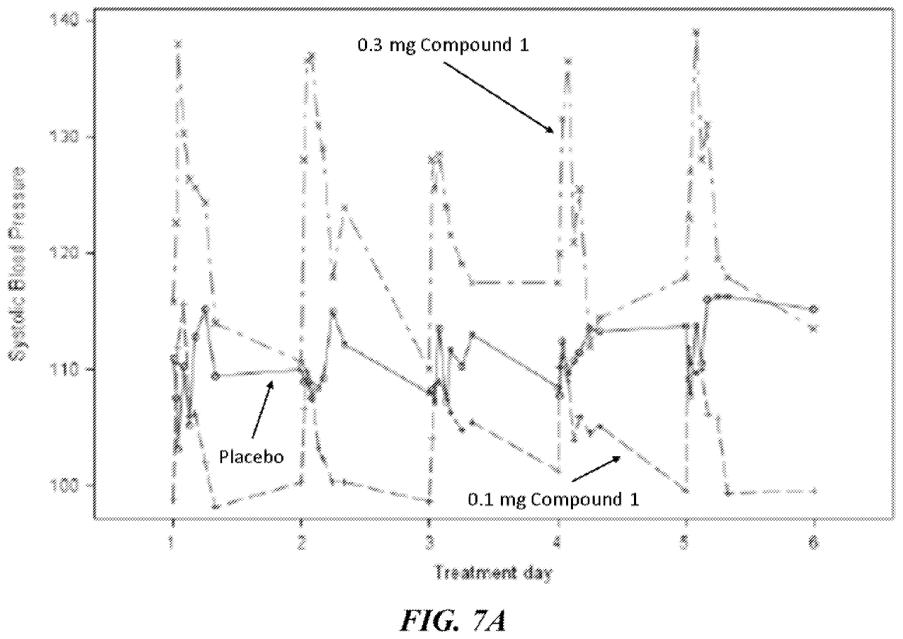

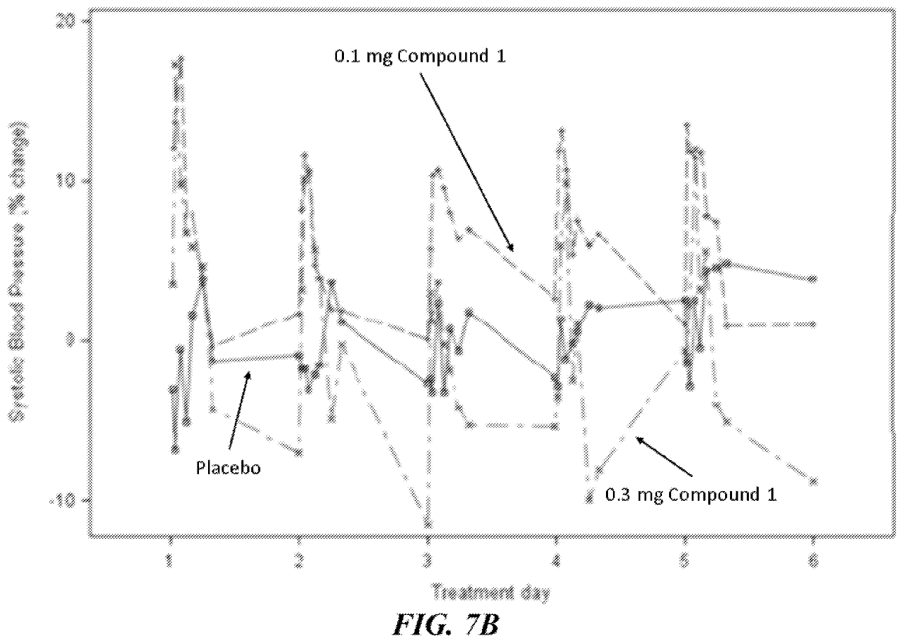

Systolic blood pressure (s.c.): After subcutaneous administration of 0.1 and 0.3 mg doses there was a reversible increase in systolic blood pressure, however less pronounced compared with diastolic blood pressure (FIG. 7A and FIG. 7B). The maximal mean increases over the 5 days of administration were 10-17 (11-18%), and 4-19 (4-17%) mmHg for the 0.1 and 0.3 mg doses, respectively, however with large inter-individual variation. The similar mean absolute levels and mean changes after each of the 5 administrations, indicated that there were no cumulative effect or sensitization/desensitization of this pharmacodynamic effect.

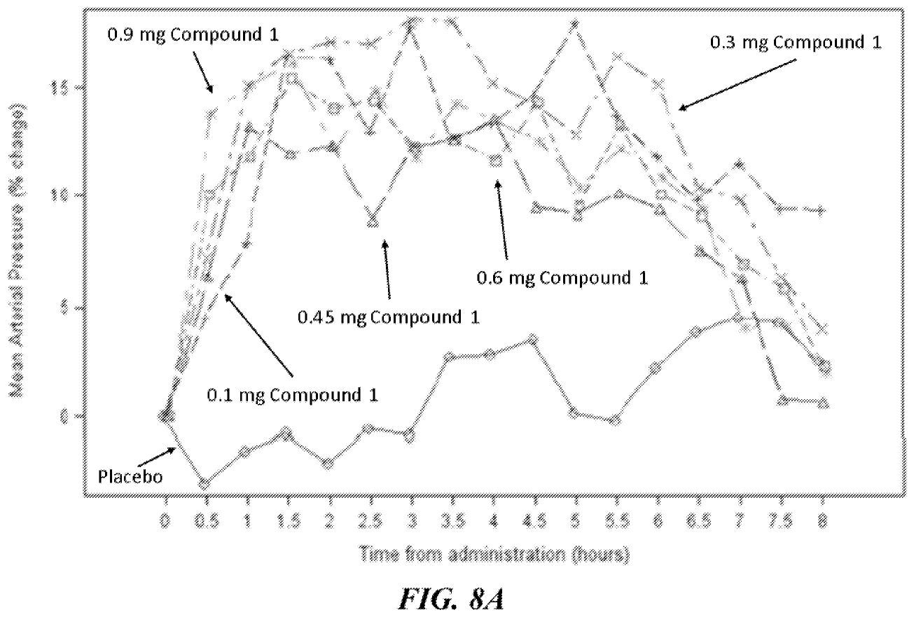

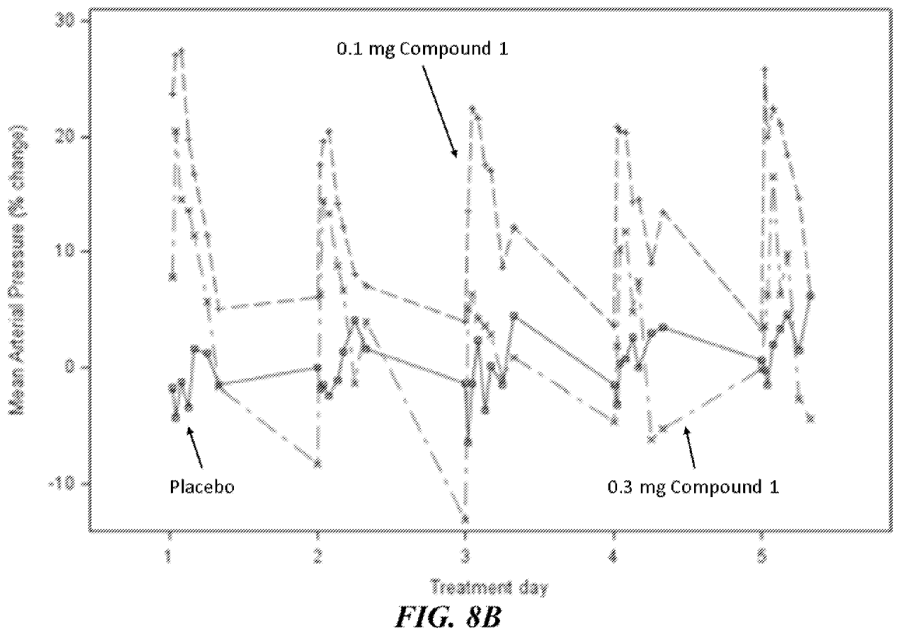

Mean arterial pressure: The overall changes in mean arterial pressure after intravenous infusion and subcutaneous injection were similar to the picture from the diastolic blood pressure (FIG. 8A and FIG. 8B) (e.g., since the latter parameter has a greater weight in the calculation than systolic pressure). The maximal mean increases after intravenous infusion were 13 (18%), 14 (18%), 12 (14%), 13 (15%), and 12 (16%) mmHg for the 0.1, 0.3, 0.45, 0.6, and 0.9 mg doses, respectively, and 14-18 (21-27%) and 6-18 (6-21%) mmHg for the 0.1 and 0.3 mg doses over the 5 days of subcutaneous administration.

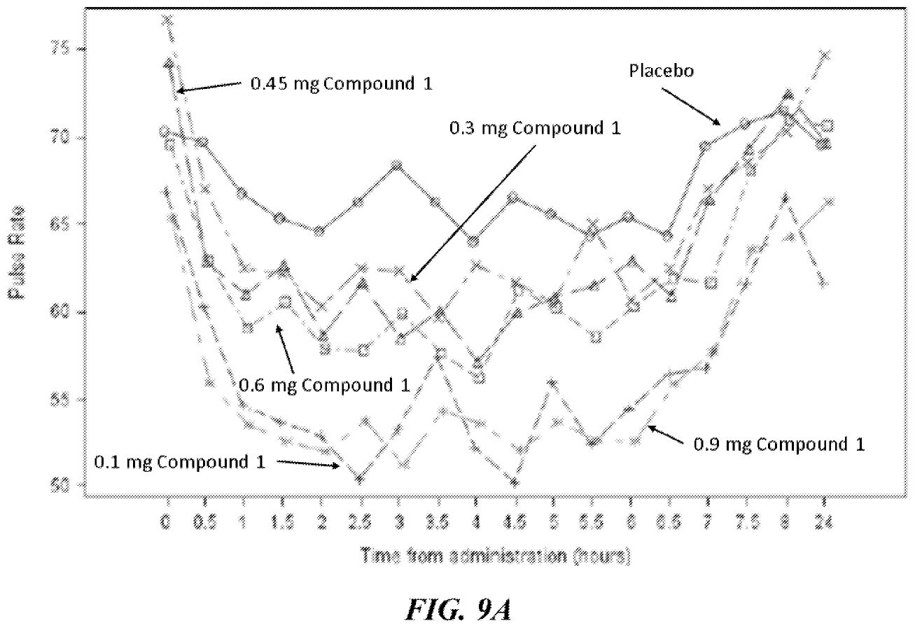

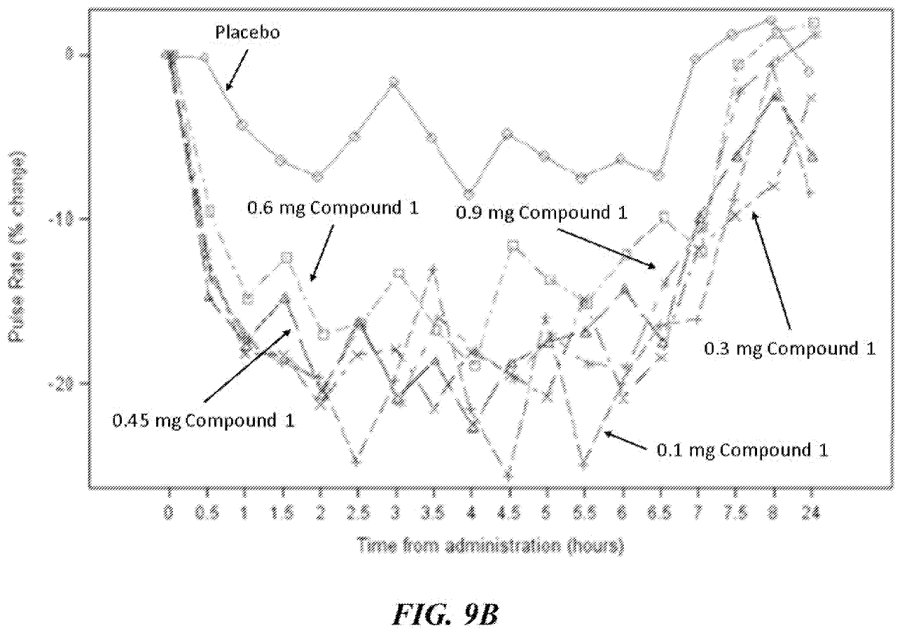

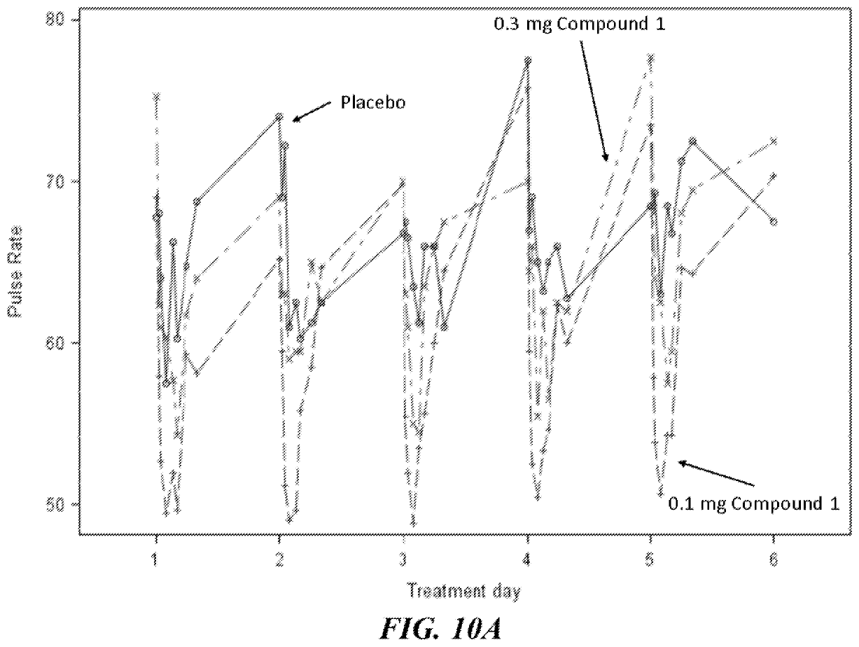

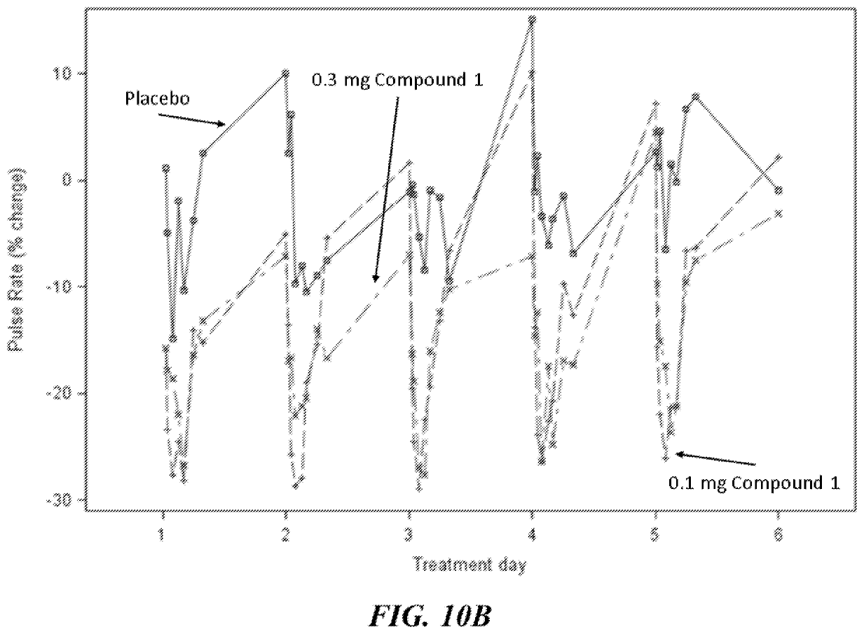

Pulse rate: A reversible decrease in pulse rate was observed after the intravenous infusion as well as after the repeated subcutaneous administrations (FIG. 9A, FIG. 9B, FIG. 10A, and FIG. 10B). The maximal mean decreases after intravenous infusion were 17 (25%), 17 (21%), 17 (23%), 13 (19%), and 14 (21%) bpm for the 0.1, 0.3, 0.45, 0.6 and 0.9 mg doses, respectively. Over the 5 days of subcutaneous administration the decrease was 18-20 (26-29%), and 16-21 (22-28%) bpm for the 0.1 and 0.3 mg doses, respectively. After end of infusion the pulse rate rapidly returned to baseline. Subjects receiving placebo treatment also showed a small decrease in the pulse rate during the infusion (FIG. 9A, FIG. 9B, FIG. 10A, and FIG. 10B).

Peripheral blood flow: Pallor was reported in 18 subjects on active treatment mainly in the 0.45, 0.6 and 0.9 mg dose groups after intravenous infusion. In general the skin color had returned to normal about 4-6 hours after the end of the intravenous infusion. Hyperemia was reported in 1 subject in the 0.45 mg dose group at 30 and 90 minutes after intravenous infusion. About 30% of the subjects in the subcutaneous dose groups, 0.1 and 0.3 mg, reported pallor after the 1st dose, the skin color had in general returned to normal 4 hours after administration. Pallor was also reported by single subjects in both dose groups during the first 2 hours after administration of the 2nd and 3rd doses, but none after the 4th and 5th administrations.

Cardiac output: The cardiac output during intravenous infusion in Period 1 was decreased in subjects on active treatment, except for subjects in the 0.1 mg dose group, compared with placebo, however with no apparent dose related trends or changes in the echocardiography parameters assessed. One event of moderate decreased cardiac output was reported as an AE after intravenous infusion in the 0.1 mg dose group. None of the changes in cardiac output met the pre-defined stopping criteria. Similarly, after subcutaneous administration a slight reduction in the parameters cardiac output was observed for subjects on active treatment in both dose groups. No abnormal values were reported for any of the echocardiography parameters assessed after repeated subcutaneous administrations.

Urine volume: There were no apparent dose related trends or changes in urinary volume after either intravenous infusion or subcutaneous injection in any of the dose groups. Large individual differences in urinary volume were seen within dose groups in both females and males.

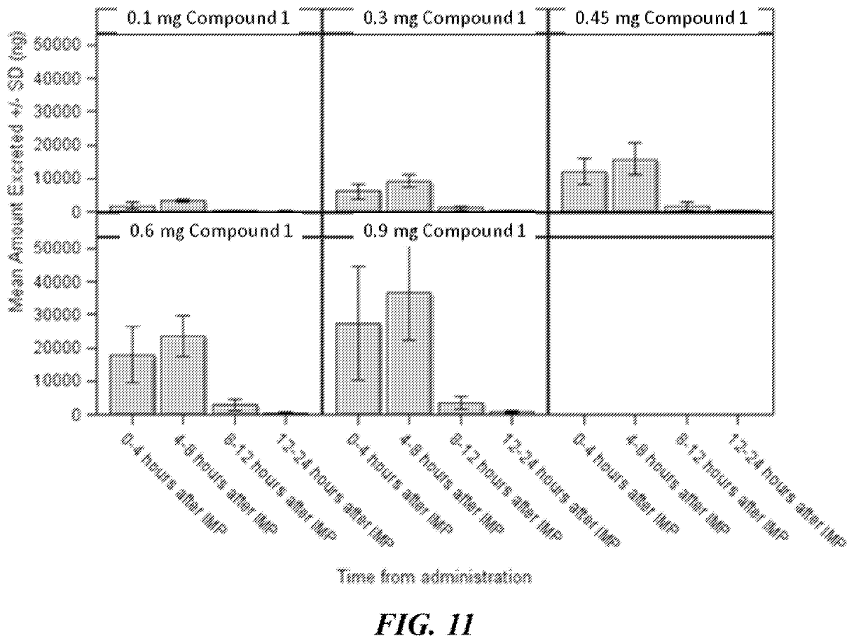

Excretion of Compound 1 in urine (i.v.): Compound 1 was excreted in urine up to 24 hours after intravenous infusion in all five dose groups. The largest amount of Compound 1 (about 3, 9, 16, 24, and 37 μg, respectively) being excreted during 4-8 hours after start of infusion in each dose group (FIG. 11). Overall, less than 10% of the dose was excreted intact in the urine after i.v. administration.

Injection site reactions: Erythema, pallor, and pruritus of mild severity were reported by several subjects on active treatment, the majority of events occurring immediately after the subcutaneous (bolus) injection. Moderate oedema and mild pain were reported by 1 subject in the 0.1 mg dose group 30 min after administration.

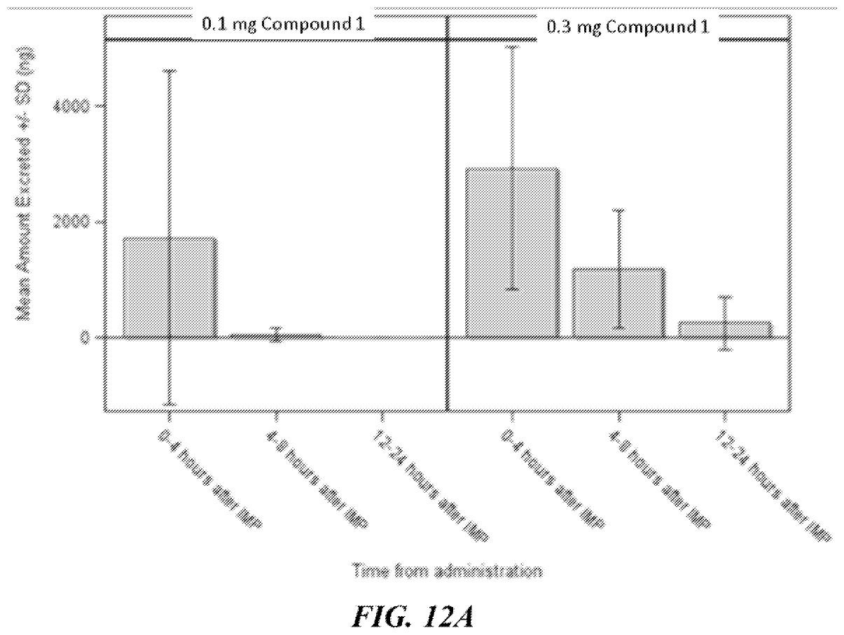

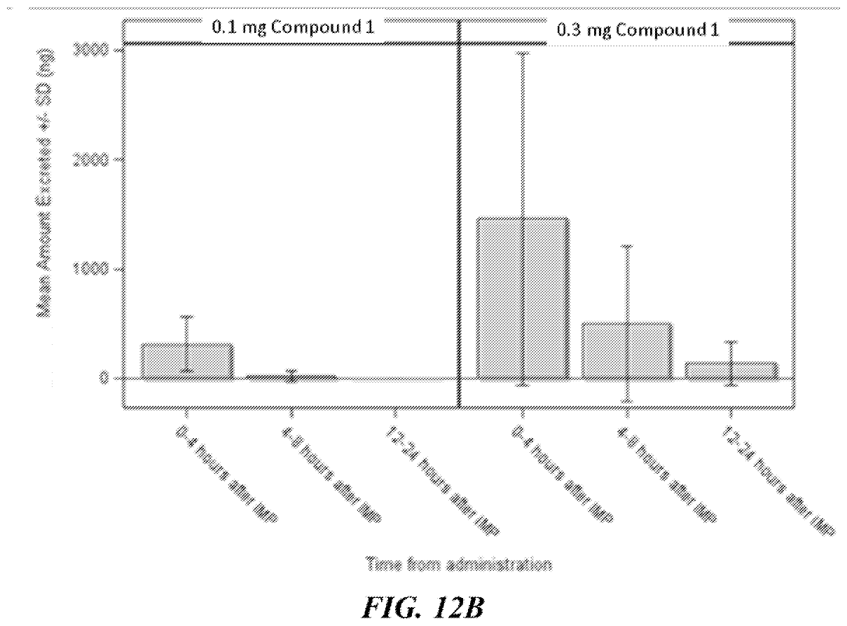

Excretion of Compound 1 in urine (s.c.): After subcutaneous injections, Compound 1 was excreted in urine up to 8 hours after injection of the 1st and 5th 0.1 mg doses, and up to 24 hours after the 1st and 5th 0.3 mg dose. The largest amount of Compound 1 was excreted during the first 4 hours after both the 1st and 5th administrations in both dose groups (FIG. 12A and FIG. 12B). After the 1st administration about 2 and 3 μg Compound 1, respectively, were excreted, and about 0.3 and 1.5 μg, after the 5th administrations (FIG. 12A and FIG. 12B). Overall less than 5% of the dose was excreted unchanged in the urine.

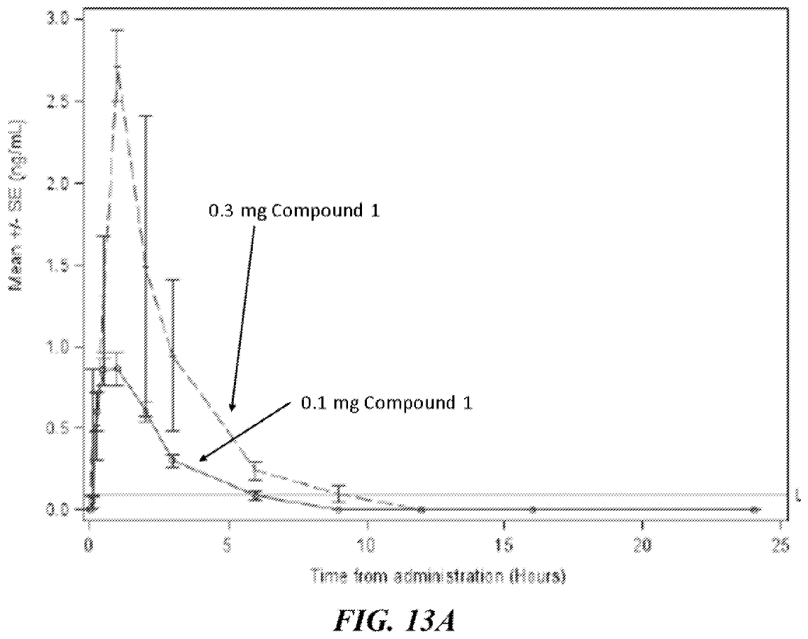

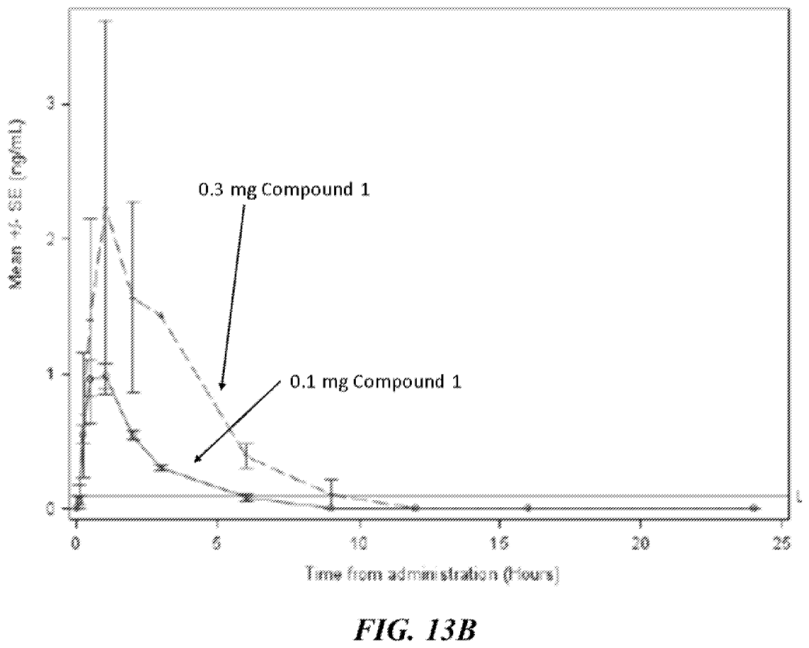

Metabolism: Analysis of human plasma from the 0.1, 0.6, and 0.9 mg i.v. dose groups and both s.c. groups using high resolution mass spectrometry indicated the presence of the active metabolite M1 (a full (V1a) agonist) after both i.v. and s.c. administrations, with only single observations of the metabolite M5. Due to analytical method interference, it was not possible to analyses for metabolites in human urine. After i.v. administration, the metabolite M1 was present only at low concentrations in one subject in the male and 4 subjects in the female dose group 0.6 mg, while it was present in all females and 5 of the 6 males in the 0.9 mg dose group, albeit with few exceptions below LOQ (FIG. 27, panel A). Mean Cₘₐₓ was just above LOQ, in the range 0.1-0.25 ng/mL, occurring at approx. 5 hours and corresponding to about 1-2% of the concomitant Compound 1 concentration with a harmonic mean terminal half-life of about 4 hours. In contrast, after s.c. administration of 0.1 mg and 0.3 mg Compound 1 the M1 concentration and exposure were comparable to Compound 1 in both dose groups (FIG. 27, panel B), after the first as well as the last administration (FIG. 13A, FIG. 13B, FIG. 3A, FIG. 3B; Table 9, cf. Table 7). The tₘₐₓ was about 0.5-1.5 hours, with a harmonic mean terminal half-life of about 1.5-2 hours. A summary of PK variables for metabolite M1 (s.c. injection) are shown in Table 9.

TABLE 9

| 1^{st} administration | | 5^{th} administration | |
|---|---|---|---|
| 0.1 mg | 0.3 mg | 0.1 mg | 0.3 mg |

AUC (h*ng/mL)

| n | 4 | | 3 | | 6 | | 1 | |
|---|---|---|---|---|---|---|---|---|
| Mean (SD) | 2.60 | (0.74) | 7.55 | (3.79) | 2.70 | (0.32) | 11.35 | (—) |
| Geometric mean (CV %) | 2.51 | (30) | 6.71 | (71) | 2.68 | (12) | 11.35 | (—) |

| | | | | | | | |
|---|---|---|---|---|---|---|---|
| Min; Max | 1.71; | 3.50 | 3.20; | 10.2 | 2.39; | 3.10 | 11.35 |

Cₘₐₓ (ng/mL)

| n | 6 | | 3 | | 6 | | 2 | |
|---|---|---|---|---|---|---|---|---|
| Mean (SD) | 0.93 | (0.22) | 2.01 | (1.24) | 1.01 | (0.23) | 2.24 | (1.95) |
| Geometric mean (CV %) | 0.90 | (23) | 1.63 | (107) | 0.98 | (24) | 1.76 | (134) |
| Min; Max | 0.70; | 1.28 | 0.60; | 2.93 | 0.74; | 1.27 | 0.86; | 3.62 |

tₘₐₓ (h)

| n | 6 | | 3 | | 6 | | 2 | |
|---|---|---|---|---|---|---|---|---|
| Mean (SD) | 0.8 | (0.6) | 0.8 | (0.3) | 0.8 | (0.3) | 1.5 | (0.7) |
| Median | 0.5 | | 1.0 | | 0.8 | | 1.5 | |
| Min; Max | 0.3; | 2.0 | 0.5; | 1.1 | 0.5; | 1.0 | 1.0; | 2.0 |

t_{1/2} (h)

| n | 4 | | 3 | | 6 | | 1 |
|---|---|---|---|---|---|---|---|
| Mean (SD) | 1.6 | (0.5) | 2.0 | (0.5) | 1.6 | (0.6) | 2.1 |
| Harmonic Mean | 1.5 | | 1.9 | | 1.4 | | 2.1 |
| Min; Max | 1.0; | 2.1 | 1.5; | 2.4 | 0.8; | 2.2 | 2.1 |

### Example 2: Potent and Selective Mixed V1AR Agonist-Antagonists

In some instances, the binding data provided herein below demonstrates Compound 1 binds to a given receptor with either its agonist portion or its antagonist portion, and that over a population of Compound 1-occupied vasopressin receptors, a fraction are occupied by the agonist portion while another fraction are occupied by the antagonist portion, such as resulting in effective partial agonism of the receptor and limiting the maximum vasoconstriction observed.

### Methods and Materials

Cell Lines. Studies were conducted with cell lines expressing rat (r) or human (h) V1a, V1b, V2, or OT receptors. For the experiments with human receptors, human embryonic kidney (HEK)-flp-in cells stably expressing the lacZ-Zeocin™ fusion gene were used for expression of hV1a and hV1b. These cells were designed for use with the Flp-In™ expression vector containing the gene of interest (here hV1a or hV1b) and the Flp recombinase expression plasmid, pOG44. For hV2 and hOTR, HEK-293 cells transiently expressing hV2 were used.

For experiments with rat receptors, A7r5 rat thoracic aorta smooth muscle cells endogenously expressing rV1a (ATCC), FLP-In 293 (HEK-293) cells stably expressing rV1b, HEK-293 cells (ATCC) transiently transfected with rV2, and Chinese hamster ovary (CHO)-K1 cells (ATCC) transiently expressing rOTR were used.

Cell Maintenance. HEK-flp-in cells were maintained in Dulbecco's modified Eagle's medium (DMEM) containing 10% (v/v) heat-inactivated fetal bovine serum (FBS), 4 mM GlutaMAX™—I, and 25 μg/mL hygromycin B at 37° C. under 5% CO₂ in a humidified atmosphere. The culture medium for hV1b-expressing cells also contained 100 U/mL penicillin and 100 μg/mL streptomycin. HEK-293 cells transiently expressing hV2 were maintained in DMEM containing 10% (v/v) heat-inactivated FBS and 4 mM L-glutamine or GlutaMAX-I at 37° C. under 5% CO₂ in a humidified atmosphere. CHO-K1 cells stably expressing hOTR were maintained in DMEM-F12 containing 5% (v/v) heat-inactivated FBS, 2 mM L-glutamine or GlutaMAX-I, and 900 μg/mL G418 sulfate at 37° C. under 5% CO₂ in a humidified atmosphere. A7r5 cells were maintained in DMEM containing 10% (v/v) heat-inactivated FBS, 4 mM GlutaMAX-1 at 3° C. under 5% CO₂ in a humidified atmosphere. On the day prior to the assay, cells were removed from culture flasks using trypsin EDTA, harvested in the medium used for cell culture, and seeded into 384-well (for V1a) or 96-well (for other receptors) poly-d-Iysine-treated plates at 7.5×10⁴ cells in 20 μL/well for rV1a, 2.5×10⁴ cells in 20 μL/well for hV1a, and 4-5×10⁴ cells in 100 μL/well for all other receptors.

Test Compounds. Compound 1 (97.3% peptide purity) and AVP (reference agonist) were used in the functional cell-based assays. Compounds were prepared in 100% DMSO as 10 mM stock concentrations (or 5 mM for AVP), stored at −20° C., and allowed to thaw just before the assay. The compounds were serially diluted to 10× working solutions in cell media. Blanks consisting of dilution media supplemented with 0.1% (v/v) DMSO were also used as controls in each study. No inhibitory effect of DMSO was seen at 0.1%. For the contractility assay, Compound 1 was formulated as a 23.5 μM stock solution in physiological salt solution (PSS; 120 mM NaCl, 4.6 mM KCI, 1.5 mM NaH₂P0₄·1H₂0, 0.7 mM Na₂HP0₄, 11.5 mM D-glucose, 25 mM NaHCO₃, 2.4 mM CaCI₂, 1.2 mM MgCI₂ [pH 7.35-7.45]). The stock solution was serially diluted in PSS to concentrations allowing for a further 1/100 dilution upon addition of the compound to the test apparatus in a cumulative fashion from lowest concentration to highest to obtain the final test concentration.

Functional Cell-Based Assays. To detect activity generated by binding of the test compounds to endogenous rV1a receptors or the stably expressed hV1a receptors, Fluorometric Imaging Plate Reader (FLlPR) calcium assays were performed. Briefly, real-time fluorescence of an intracellular calcium-sensitive dye was measured immediately upon addition of the test compound at various concentrations. The endogenous ligand of V1a, AVP, was used as the reference agonist. Reporter gene assays were used to monitor agonist-induced activity at the human and rat V1b, V2, and OT receptors. Cells expressing the receptor of interest were transiently transfected with a luciferase reporter gene under the control of transcriptional regulatory elements responsive to receptor activation. Expression of the luciferase gene was determined after 5-h incubation with various concentrations of test compound. AVP was used as the reference agonist in V1b assays, desmopressin (dDAVP) was used as the reference agonist in V2R assays, and carbetocin was the reference agonist in the OT receptor assays. For V1a receptor response, area under the curve of the real-time calcium traces, expressed as relative fluorescence units, was determined. For V1b, V2, and OT receptor response, luciferase activity was expressed in luminescent counts per second. Compound potency was expressed as the concentration that produced a half-maximal response (EC₅₀), calculated by a four-parameter non-linear regression analysis of concentration-response curves using ActivityBase™ software. Efficacy was expressed in relative terms as percent maximal possible effect (% MPE) relative to the maximal response of the reference agonist for each assay (AVP for V1a and V1b, dDAVP for V2R, and carbetocin for OTR).

Arterial Contractility Assay. Resistance arteries were isolated from human mesenteric tissue and finely dissected under a stereomicroscope while bathed in carbogen-aerated PSS maintained at 37° C. Arterial segments (2 mm) were mounted in a pressure myograph system between two glass cannulae in 7-mL tissue baths containing aerated PSS. Arteries were gradually pressurized to 60 mmHg. Arterial contractility was stabilized by potassium-induced depolarization with three consecutive exposures to a solution with a high potassium concentration (PSS with 124.34 mM KCI and no NaCl; 124K+PSS), each followed by a wash of the bath with aerated PSS. Cumulative concentration-response curves (CCRC) were then generated for the compounds. Arteries were bathed in aerated PSS containing the initial compound concentration (0.1 nM Compound 1), and each following dose was added to the bath without draining, taking into account the amount of compound already in the chamber when calculating the final concentration. The concentrations used were 0.1, 0.3, 1, 3, 10, 32, and 100 nM Compound 1. Only a single CCRC was generated in each arterial segment tested. Contractile activity was determined by measuring the arterial outer diameter via digital video edge detection in response to each 124K+PSS depolarization and each compound concentration. Throughout the experiment, vessel diameter data were collected continuously using DMT Vessel Acquisition Suite software. For each 124K+PSS stimulation or compound concentration, data were collected until the vessel diameter was judged to have reached a plateau before proceeding to the next experimental step. The vessel diameters for each artery preparation (in millimeters) were analyzed by using a Microsoft Excel template. Data collected in response to the three 124K+PSS depolarization cycles were first used to determine artery stability. The contraction induced by the last stimulation (3rd 124K+PSS) was then used as the internal reference response of each artery (ie, 100% contraction) for reporting contractile activity. Arteries that could not be stabilized with three cycles of depolarization-induced contraction were excluded from the study.

### Results

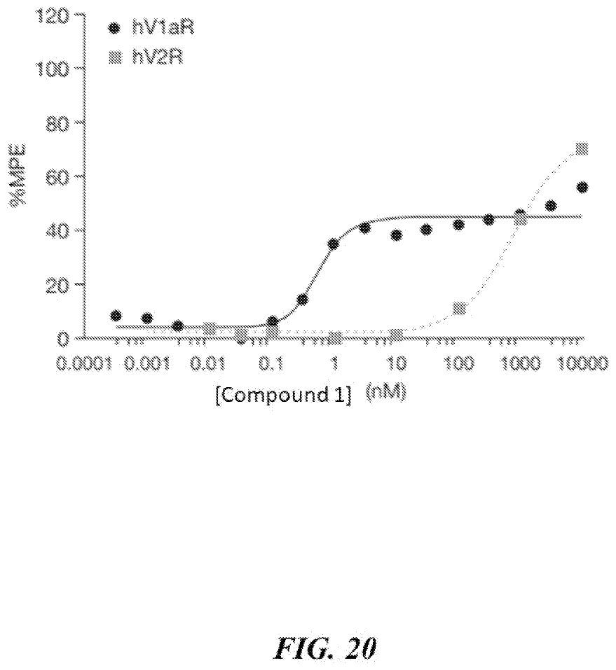

Functional Cell-Based Assays: When the effect of Compound 1 on hV1a receptors was examined, the activity reached a mean of 39% MPE relative to the activity with AVP, with a mean EC₅₀ of 0.71 nM (Table 14), which indicates that Compound 1 acts as a partial agonist at V1a receptors. The % MPE plateaued at a concentration of ˜1 nM Compound 1, with no further increases in % MPE in response to further increases in concentration (FIG. 19). The partial agonism indicates that treatment with Compound 1 results in vasoconstriction of the splanchnic vasculature in individuals with portal hypertension, such as reducing portal blood flow and pressure and improving the patient's systemic hemodynamics with a lower risk of ischemia than a full (V1a) agonist. In addition, Compound 1 was selective for hV1a receptors at a wide range of concentrations, with much higher EC₅₀ at hV1b, hV2, and hOT receptors than at V1a receptors (Table 14). Similar results were seen for potency and activity at rat vasopressin receptors (Table 19). The dose-response curves of Compound 1 activity at hV1a and hV2 receptors also differed, with Compound 1 showing ˜600-fold more potency at hV1a than at hV2 (FIG. 20). While Compound 1 elicited a higher maximal response at V2 (74% MPE) than at V1a receptors (39% MPE), this occurred at concentrations about 1000 times higher than the lowest concentration that elicited the maximal response at V1a receptors (FIG. 20), where no V2 response was observed. As such, at clinically relevant concentrations, Compound 1 has little to no activity at human V2 receptors.

TABLE 14

| Recep- | Test | EC₅₀ (nM) | | | % MPE | | |
|---|---|---|---|---|---|---|---|
| tor | compound | Mean | 95% CI | N | Mean | 95% CI | N |
| hV1a | Com- | 0.71 | 0.45-1.1 | 12 | 39 | 31-47 | 13 |
| | pound 1 | | | | | | |
| | AVP | 0.07 | 0.06-0.08 | 278 | 100 | — | 284 |
| hV1b | Com- | 134 | 92-194 | 9 | 72 | 66-78 | 9 |
| | pound 1 | | | | | | |
| | AVP | 4.3 | 3.9-4.7 | 347 | 100 | — | 360 |
| hV2 | Com- | 420 | 278-634 | 13 | 74 | 67-82 | 13 |
| | pound 1 | | | | | | |
| | AVP | 0.05 | 0.04-0.07 | 120 | 95 | 92-97 | 137 |
| hOTR | Com- | 6.9 | 4.5-11 | 6 | 33 | 29-37 | 8 |
| | pound 1 | | | | | | |
| | AVP | 15 | 9.1-25 | 4 | 106 | 81-130 | 4 |

TABLE 15

| | Test | EC₅₀ (nM) | | | % MPE | | |
|---|---|---|---|---|---|---|---|
| Receptor | compound | Mean | 95% CI | N | Mean | 95% CI | N |
| rV1a | Com- | 0.22 | 0.06-0.83 | 7 | 30 | 25-35 | 7 |
| | pound 1 | | | | | | |
| | AVP | 0.08 | 0.06-0.09 | 197 | 100 | — | 206 |
| rV1b | Com- | 75 | 43-130 | 8 | 97 | 86-109 | 8 |
| | pound 1 | | | | | | |
| | AVP | 16 | 14-18 | 37 | 100 | — | 38 |
| rV2 | Com- | 4.6 | 4.2-5.0 | 13 | 83 | 71-94 | 13 |
| | pound 1 | | | | | | |
| | AVP | 0.04 | 0.03-0.05 | 67 | 98 | 96-101 | 63 |
| rOTR | Com- | 2.1 | 1.1-4.0 | 6 | 95 | 79-111 | 6 |
| | pound 1 | | | | | | |
| | AVP | 0.93 | 0.48-1.8 | 10 | 82 | 74-160 | 17 |

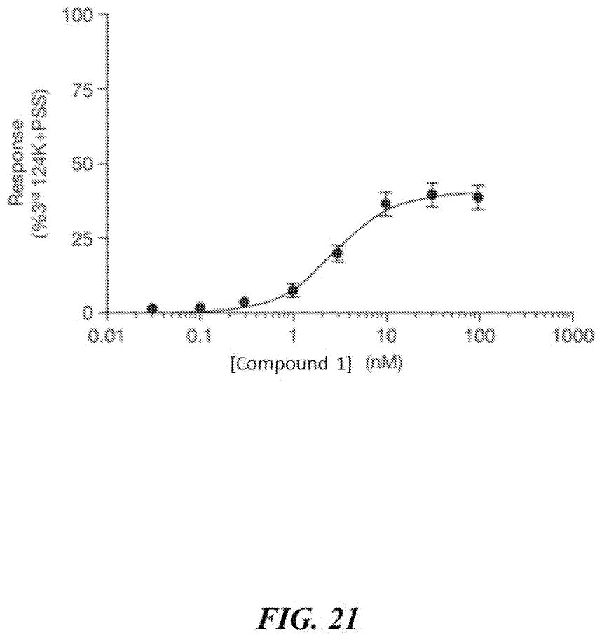

Arterial Contractility Assay: Treatment of human mesenteric resistance arteries with Compound 1 resulted in an attenuated maximal response (40.0%) relative to the maximal depolarization achieved with potassium (expected to be close to the contraction elicited by the endogenous ligand, AVP) (FIG. 21), which helps confirm that Compound 1 acts as a partial agonist at V1a receptors.

### Example 3: M1 Formation Decreases After SC Infusion

In an effort to reduce metabolite (M1) formation after subcutaneous administration, a compound described herein (e.g., Compound 1) was administered to healthy individuals by subcutaneous infusion. In contrast to subcutaneous (bolus) injection, little to no formation (e.g., less than 20%) of the metabolite M1 occurred after a composition comprising a compound described herein (e.g., Compound 1) was subcutaneously infused into healthy rats and minipigs. The results provided herein demonstrate that metabolite (M1) formation can be substantially reduced by subcutaneously infusing a composition comprising a compound described herein (e.g., Compound 1).

### Protocol—Single Dose Subcutaneous Infusion Study in Rats and Mini-Pigs

A composition comprising 6 milligrams/milliliters (mg/mL) of Compound 1 was continuously infused (subcutaneously via mini-osmotic pump) into 36 rats over a period of 24 hours. Each rat was administered 200 microliters (μL) of the composition, for a total dose of 4.8 mg/kg of Compound 1. The incision for the mini-pump was on the cranial right side of the spine, a trocar or hemostats may be inserted to create a subcutaneous pocket, and the pump inserted into the subcutaneous pocket with the delivery port of the mini-pump pointing cranially. The doses were given into mid-dorsal area via surgical implantation of the pump filled with test article. Following insertion of the implant, a 4 cm×4 cm area surrounding the pump will be delineated and designated as the test site. The test site will be delineated and remarked as necessary thereafter.

Blood samples were collected from each rat at various timepoints, including 0.25 hour (hr), 1 hr, 3 hr, 7 hr, 24 hr, 25 hr, 27 hr, and 29 hr. Plasma samples were analyzed for concentration of Compound 1 and M1 using a validated analytical procedure.

A similar approach was used to evaluate concentration of Compound 1 and M1 in mini-pigs after subcutaneous infusion.

### Results

Generally, the subcutaneous infusion studies in rats and mini-pigs demonstrated that M1 formation was greatly reduced (compared to SC (bolus) injection) after SC infusion of Compound 1. Surprisingly, less than about 20% of Compound 1 was converted to M1 after SC infusion in rats and mini-pigs. No relevant adverse events were reported.

Specifically, little to no M1 formed after subcutaneous infusion of Compound 1 into healthy mini-pigs (FIGS. 45-48). Moreover, M1 formation was either relatively higher within the first 24 hours of treatment or consistently low throughout the duration of the study (FIGS. 45-48). Surprisingly, while tripling the dose of Compound 1 from about 20 microgram per hour per kilogram (μg/hr/kg) to about 60 μg/hr/kg did not substantially reduce M1 formation in mini-pigs (compare FIG. 45, panel A to FIG. 45, panel C), a substantial reduction in M1 formation was observed in mini-pigs when the dose of Compound 1 was tripled and the infusion rate was lowered (compare FIG. 46 to FIG. FIG. 48, panel B). Moreover, lowering the infusion rate at a lower dose reduced M1 formation within the first 24 hrs (compare FIG. 46 to FIG. 48, panel A). As such, these results demonstrate that little to no M1 is formed when Compound 1 is administered at higher doses and lower infusion rates, and that further reduction in M1 formation can be achieved by decreasing the subcutaneous infusion rate.

Overall, the results described herein demonstrate that subcutaneous infusion is an alternative subcutaneous administration route that can used to reduce M1 formation in mammals.

### Example 4: Subcutaneous Formulation Development

### Buffer Selection and Buffer Concentration

As discussed in Example 1 above, metabolite (M1—a full (V1a) agonist) concentration and Compound 1 concentration after SC (bolus) injection in healthy volunteers were found to be about equimolar (FIG. 44, panel B). In contrast, little to no M1 was formed after IV infusion of Compound 1 into healthy volunteers (FIG. 44, panel A). Formulation(s) that reduced M1 formation are described herein.

To evaluate how buffer concentration affects degradation of partial V1a agonist-antagonists described herein (e.g., Compound 1), such as in the presence of trypsin, the buffer concentration of the subcutaneous formulation was increased. Generally, higher buffer concentrations, such as concentrations of about 100 mM, provided substantially higher percent recovery of the parent compound compared to lower buffer concentrations, such as concentrations of about 10 mM. In some instances, compositions having a buffer concentration of about 10 mM or more provide a percent recovery of the parent compound of about 60% or more. In some instances, compositions having a buffer concentration of about 100 mM or more provide a percent recovery of the parent compound of about 90% or more.

### Protocol

To mimic the diluted concentration of the composition in a local subcutaneous environment (e.g., post-subcutaneous injection), a conservative method for the effect of pH and buffer concentration was used.

For instance, compositions comprising Compound 1 was formulated with mannitol, acidic buffer (acetate, succinate, citrate, etc.) to the target pH (e.g., pH of 3-7). A 6× dilution with phosphate buffer pH 7.0 was used to simulate 1 volume of body fluid from each direction of the injected volume (top, bottom, left, right, front and back). Trypsin was added to each test well. Each test well was incubated at 37° C. for 30 minutes. Each test well was then quenched with formic acid and analyzed for parent percent (%) recovery and % (trypsin) inhibition.

All formulations were diluted 1 to 7 with PBS at pH: 7.4 to simulate subcutaneous environment during injection.

### Results

The percent recovery and percent trypsin inhibition for acetate, succinate, and citrate buffers are shown in Table I-A and Table I-B.

The results indicate that a composition having a pH of about 4-4.5 and comprising at least about 30 mM succinate buffer provide a percent recovery of the parent compound (e.g., Compound 1) of about 75% or more. Additionally, the results indicate that a composition having a pH of about 4-4.5 and comprising at least about 30 mM citrate buffer provide a percent recovery of the parent compound (e.g., Compound 1) of about 70% or more. Moreover, the results indicate that a composition having a pH of about 4-4.5 and comprising at least about 30 mM of acetate buffer provide a percent recovery of the parent compound (e.g., Compound 1) of about 60% or more.

Overall, the results indicate that a higher buffer concentration (e.g., 100 mM) increases percent recovery of the parent compound in subcutaneous compositions described herein. For example, recovery of Compound 1 at buffer concentrations of about 100 mM provided a percent recovery of about 80% or more, such as precent recoveries of Compound 1 being greater than 90% in some cases. Generally, trypsin was inhibited more in environments having a larger buffer concentration.

TABLE I-A

| | | pH (Pre- | % | % |
|---|---|---|---|---|
| Formulation | Buffer Strength and pH pre-dilution | digest) | Recovery | Inhibition |
| 1 | Undiluted 10 mM phosphate (pH 7.0) | 6.87 | 53.5 | n.d. |
| | 10 mM Acetate (pH 4.5) | 6.98 | 49.2 | −4.3 |
| 2 | | | | |
| 3 | 30 mM Acetate (pH 4.5) | 6.67 | 60.2 | 6.7 |
| 4 | 50 mM Acetate (pH 4.5) | 6.38 | 66.4 | 12.9 |
| 5 | 100 mM Acetate (pH 4.5) | 5.5 | 84.3 | 30.8 |
| 6 | 10 mM Acetate (pH 4.0) | 6.92 | 52 | −1.5 |
| 7 | 30 mM Acetate (pH 4.0) | 6.49 | 64.5 | 11 |
| 8 | 50 mM Acetate (pH 4.0) | 6.03 | 75.8 | 22.3 |
| 9 | 100 mM Acetate (pH 4.0) | 4.97 | 92.1 | 38.6 |

n.d.: not determined

TABLE I-B

| | | pH | pH (Pre- | % | % |
|---|---|---|---|---|---|
| Formulation | Buffer Strength | (Formulation) | digest) | Recovery | Inhibition |
| 10 | 10 mM Phosphate | 7 | 6.82 | 51.5 | n.d. |
| 11 | 10 mM Sodium Succinate | 4.59 | 6.81 | 51.9 | 0.4 |
| 12 | 30 mM Sodium Succinate | 4.57 | 6.26 | 67.4 | 15.9 |
| 13 | 50 mM Sodium Succinate | 4.51 | 5.71 | 79.2 | 27.7 |
| 14 | 100 mM Sodium Succinate | 4.5 | 5.14 | 90.0 | 38.5 |
| 15 | 10 mM Sodium Succinate | 4.07 | 6.72 | 57.4 | 6.0 |
| 16 | 30 mM Sodium Succinate | 4.01 | 5.91 | 78.1 | 26.6 |
| 17 | 50 mM Sodium Succinate | 3.98 | 5.27 | 88.0 | 36.5 |
| 18 | 100 mM Sodium Succinate | 3.99 | 4.7 | 95.1 | 43.6 |
| 19 | 10 mM Phosphate | 6.95 | 6.82 | 43.3 | n.d. |
| 20 | 10 mM Sodium Citrate | 4.48 | 6.65 | 47.3 | 4.0 |
| 21 | 30 mM Sodium Citrate | 4.5 | 6.04 | 65.6 | 22.3 |
| 22 | 50 mM Sodium Citrate | 4.51 | 5.59 | 76.5 | 33.2 |
| 23 | 100 mM Sodium Citrate | 4.5 | 5.15 | 84.9 | 41.6 |
| 24 | 10 mM Sodium Citrate | 4 | 6.55 | 48.1 | 4.8 |
| 25 | 30 mM Sodium Citrate | 4 | 5.73 | 72.0 | 28.7 |
| 26 | 50 mM Sodium Citrate | 4.01 | 5.17 | 85.5 | 42.2 |
| 27 | 100 mM Sodium Citrate | 4 | 4.67 | 92.7 | 49.4 |

M1 Formation Decreases after SC Administration with Increasing Buffer concentration

As discussed above, while undesirable metabolites (e.g., M1) can form after subcutaneous administration of a compound described herein (e.g., Compound 1), formation of such metabolites can be substantially reduced (in the presence of trypsin) by using higher buffer concentrations.

To evaluate how buffer concentration of the composition affects degradation of partial V1a agonist-antagonists (e.g., Compound 1) in mammals, such as in rats, compositions comprising relatively high or low buffer concentration were subcutaneously (bolus) injected to rats. Generally, higher buffer concentrations, such as concentrations of about 100 mM, provided substantially reduced metabolite (e.g., M1) formation than compositions comprising lower buffer concentrations, such as concentrations of about 10 mM. Moreover, higher buffer concentrations, such as concentrations of about 100 mM, generally provided substantially higher bioavailability of the parent compound compared to lower buffer concentrations, such as concentrations of about 10 mM.

### Results

The results indicate that a composition having a pH of about 4-4.5 and comprising about 100 mM or more of a buffer (e.g., acetate buffer, succinate buffer, or citrate buffer) substantially improved the bioavailability of the parent compound (e.g., Compound 1) after subcutaneous (bolus) injection compared to a composition having the same pH but a buffer concentration of about 10 mM.

Additionally, the results indicate that a composition having a pH of about 4-4.5 and comprising about 100 mM or more of a buffer (e.g., an acetate buffer, a succinate buffer, or a citrate buffer) substantially reduced metabolite (e.g., M1) formation, generally, after subcutaneous (bolus) injection compared to a composition having the same pH but a buffer concentration of about 10 mM.

Overall, the results demonstrate that a composition comprising a relatively high buffer concentration (e.g., 100 mM) have a better PK profile (e.g., bioavailability) for the parent compound (e.g., Compound 1) than compositions comprising relatively low buffer concentration (e.g., 10 mM). For example, after a composition having a buffer concentrations of about 10 mM is administered by subcutaneous (bolus) injection, the bioavailability of Compound 1 is about 13%. In contrast, after a composition having a buffer concentrations of about 100 mM is administered by subcutaneous (bolus) injection, the bioavailability of Compound 1 is about 37%. Additionally, the relative amount of M1 formed after a composition having a buffer concentrations of about 10 mM is administered by subcutaneous (bolus) injection is at least about 15% more than the relative amount of M1 formed after a composition having a buffer concentrations of about 100 mM is administered by subcutaneous (bolus) injection. In some cases, the relative amount of M1 formed is about 50% less after compositions comprising relatively high buffer concentrations (compared to compositions having lower buffer concentrations) are administered by subcutaneous (bolus) injection.

### Preservatives

Additives, such as preservatives, can extend stability (e.g., shelf-life) of drug formulations, such as subcutaneous formulations described herein. In contrast, intravenous compositions often do not require preservatives since they are formulated on-site (e.g., shortly before the composition is administered).

### Protocol

Four preservatives (m-cresol, phenol, chlorobutanol, and benzyl alcohol) were screened in short-term stability studies at 40° C. for six weeks, as measured by HPLC chromatogram analysis. Specifically, the preservatives were added to 5 milligram per milliliters (mg/mL) or 10 mg/mL composition having a pH of about 4.5 and comprising about 10 mM sodium acetate and about 43.6 mg/mL mannitol. The resulting compositions were evaluated by recovery, purity, degradation, and physical stability. In some instances, the recovery, purity, degradation, and physical stability were compared to a control composition having a pH of about 4.5 and comprising about 10 mM sodium acetate and about 43.6 mg/mL mannitol.

### Results

Among m-cresol, phenol, chlorobutanol, and benzyl alcohol, m-cresol had a better concentration recovery and purity profile during the six-week period. For example, when the concentration of the composition was increased to 10 mg/mL, m-cresol maintained similar concentration recovery and purity while the other three preservatives exhibited lower concentration recovery and purity. There were slightly more existing impurities in the m-cresol and phenol formulations compared to the control composition at 40° C. for two weeks. The chlorobutanol and benzyl alcohol formulations exhibited higher levels of existing degradation products and impurities at 40° C. for two weeks. No new impurities were observed in the m-cresol and phenol formulations at 40° C. for two weeks. In contrast, new impurities were observed in the chlorobutanol and benzyl alcohol formulations at 40° C. for two weeks. All formulations were clear and colorless at 40° C. for six weeks. M-cresol was selected as the preservative for subcutaneous (e.g., bolus or infusion) compositions described herein.

### Example 5: Pharmacokinetic and Pharmacodynamic Analysis of Mixed V1AR Agonist-Antagonist in Animals

In general, the PK/PD results in healthy rats and monkeys described herein provides additional support of the mixed agonist-antagonist activity of the compounds described herein. Specifically, after both IV and SC (bolus) injection, a compound described herein (e.g., Compound 1) was systemically delivered to both healthy rats and monkeys, as demonstrated by the reported systemic effects (e.g., change in mean arterial pressure (MAP)). Moreover, injection site reactions, such as redness, skin irritation, erythema, or scab, were observed in individuals administered the compound by SC (bolus) injection. Such injection site reactions can be explained by formation of a full agonist described herein (e.g., M1) at the injection site, further highlighting the undesirability of M1 formation upon subcutaneous administration of a compound described herein.

### Materials and Methods

### Pharmacokinetics in Healthy Rats

Rat models were used as a model to assess PK/PD of Compound 1. The studies were each performed using four adult male Sprague Dawley rats housed in a controlled environment with free access to food and water for >3 days before experimentation. Compound 1 was dissolved in 5% mannitol and administered <2 h after IV and SC administration of formulation in rats for both studies.

### Intravenous (IV) Administration

The four rats were catheterized via the jugular vein (for compound administration) and carotid artery (for blood collection). A solution containing three compounds, including Compound 1 (cassette dosing), was injected into the jugular vein catheter (0.1 mg/mL of each compound, 0.3 mL per animal; nominal dose of 0.1 mg/kg Compound 1). Blood samples were collected at 2, 6, 10, 15, 20, 30, 45, 60, 90, and 120 min after administration using an automated blood sampling system (Instech Laboratories Automated Blood Sampling Unit 2nd generation).

### Subcutaneous (SC) Administration

Four adult male Sprague Dawley rats were catheterized via the carotid artery (for blood collection). Three compounds, including Compound 1, were individually administered by SC bolus into the mid-lumbar region on the dorsal aspect of each animal (parallel individual administration of each compound, 1.0 mg/mL, 0.3 mL per animal; nominal dose of 1.0 mg/kg Compound 1). Blood samples were collected at nominal times of 5, 10, 20, 40, 60, 90, 120, 180, 240, and 300 min after administration using an automated blood sampling system as described above. Plasma was prepared from whole blood using K₂EDTA as anticoagulant. Subsequent bioanalysis of samples included compound extraction and plasma concentration determination using standard LC/MS methods.

### Pharmacodynamics in Healthy Rats

### Skin Blood Flow and Blood Lactate

This study was performed over multiple experiments using a total of 48 adult male Wistar rats. Compounds were dissolved in 2.5% dextrose and 0.45% sodium chloride on the day of the study, at concentrations allowing administration of the desired dose at an infusion rate of 20 μL/kg/min (0.15-150 μmoL/μL). Compounds were infused via an IV catheter at six sequentially increasing doses. A group of vehicle-administered animals received vehicle throughout the study and data were reported from intervals timed analogously with the dose-response in compound 1-administered animals.

On the day of experimentation animals were anesthetized and instrumented. A tracheostomy was performed to allow assisted respiration and three catheters were placed. Laser Doppler probes were positioned on a shaved portion of the animal's lower abdominal skin for blood-flow measurements. Ventilation was initiated, and baseline readings for the primary parameter of skin blood flow (SBF) were recorded. Readings for the secondary parameters were also recorded: (1) blood lactate concentration, (2) mean arterial pressure (MAP), and (3) blood pH. Data collection was performed continuously for SBF and MAP using NOTOCORD-hem™ (Instem), and blood samples were taken at each compound dose and immediately analyzed using an i-STAT® meter.

After the final dose administration, animals were euthanized and a final SBF measurement was recorded to detect any signal from the flow probe after the animal's heart stopped beating to control for a signal not attributed to blood flow. Animal body temperature, pulse, and ventilation adequacy were monitored throughout the procedure.

### Vitals

The study was performed using six adult male Sprague Dawley rats, aged ˜11 weeks. The pressure catheter was a polyurethane tubing that extended out of the device body and was inserted into the lower abdominal aorta. A postoperative injection of analgesia (flunixin, 5 mg per animal; Finadyne®, Schering-Plough) was given once immediately after implantation, and once at an interval of 24 h. The surgical wounds were disinfected with povidone iodine (Vétédine®, Vetoquinol SA) for 4 consecutive days. After surgery, the animals recovered for ˜2 weeks before first study administration (day 0).

Animals were allocated at random using a Latin-square design and administered according to the schedule in Table 16.

TABLE 16

| | Animal | | | | | |
|---|---|---|---|---|---|---|
| | 1 | 2 | 3 | 4 | 5 | 6 |
| Day 0 | Control | DL1 | DL2 | DL3 | DL4 | Control |
| Day 4 | DL1 | DL2 | DL3 | DL4 | Control | DL1 |
| Day 7 | DL2 | DL3 | DL4 | Control | DL1 | DL2 |
| Day 11 | DL3 | DL4 | Control | DL1 | DL2 | DL3 |
| Day 14 | DL4 | Control | DL1 | DL2 | DL3 | DL4 |

Control = 0 mg/kg/day;
DL1 = 0.1 mg/kg/day;
DL2 = 0.6 mg/kg/day;
DL3 = 4.0 mg/kg/day;
DL4 = 20 mg/kg/day,
DL, dose level

Animals were housed in a controlled environment with free access to food and water for ≥8 days before experimentation and fasted for ≥16 h before surgery for telemetry (anesthetized by continuous inhalation of isoflurane; induction and maintenance of anesthesia in oxygen). There were ≥13 days between telemetry surgery and the first day of administration. The transmitter body was implanted under aseptic conditions into the abdominal cavity.

Control administration was performed with vehicle #2, sterile physiological saline (0.9% NaCl). Compound 1 for DL4 was prepared as a stock solution in 0.1% (v/v) acetic acid in water for injection. Animals administered with DL1, DL2, and DL3 received the stock solution diluted with sterile physiological saline (0.9% NaCl) to achieve the requested concentrations.

Animals were observed at least twice daily. On the days of administration, animals were examined before and immediately after administration, and at least once after dosing (after the ≥4-h analysis time point) to detect any clinical signs or reaction to administration. A full clinical examination was performed weekly.

NOTOCORD-Hem™ software was used for collection and analyses of hemodynamic parameters and body temperature; body temperature and arterial blood pressure signals were continuously sampled at 50 and 500 Hz, respectively. Body temperature and hemodynamic parameters were recorded in all animals, on days 0, 4, 7, 11 and 14, starting >1.5 h before administration and for ≥24 h after administration.

### Telemetry Information for Healthy Rat PD Vitals

Telemetric system consisted of the following (DSI; St. Paul, MN) implantable device (model TL11M2-C50-PXT), signal receiver (model RPC-1, located on the bottom of each cage), data exchange matrix (model DEM centralizing signals from all animals), ambient pressure reference (model APR-1, allowing a barometric correction), and microcomputer (PC-type with acquisition card).

Pharmacokinetics and Pharmacodynamics in Healthy Monkeys (n=3)

The studies of IV and SC Compound 1 administration were each performed using three adult male cynomolgus monkeys. For both studies, Compound 1 was dissolved in saline (0.9% NaCl) and administered within 2 h post formulation. Clinical observations were made before and after dosing. IV administration

Animals were monitored daily by the technical staff for any signs of illness or distress. Compound 1 was bolus administered via the catheter (0.49 mg/mL, 0.5 mL per animal, nominal doses of 0.05 mg/kg) which was flushed immediately after injection with saline (˜1 mL). Animals were food and water fasted prior to dosing and until >3 h after dosing. Blood samples (0.5 mL in EDTA) were collected from a femoral or saphenous vein before dosing and at times continuing out to 180 min after administration.

### SC Administration

The study was performed using three adult male cynomolgus monkeys. The monkeys were weighed and Compound 1 was bolus administered cage-side into the animal's dorsal back (4.88 mg/mL, 0.5 mL per animal, nominal doses of 0.5 mg/kg). Animals were food and water fasted prior to dosing and until ≥3 h after dosing.

Blood samples (0.5 mL in EDTA) were collected from a femoral or saphenous vein before dosing and at multiple times continuing out to 480 min after administration. Lactate concentration was also measured as part of PD analysis and collected as EDTA whole blood from the pre-dose, 15-, 40-, 90-, 180-, and 360-min post-administration samples immediately after collection using a Lactate Plus (Nova Biomedical) meter. For blood sample analysis, plasma was prepared from whole blood. Subsequent bioanalysis included compound extraction followed by standard LC/MS methods to determine compound concentration in each sample.

Pharmacodynamics in Healthy Monkeys, Compound 1 versus Terlipressin (n=6)

The in-life portion of the study was performed over multiple dosing sessions using six non-naive adult male cynomolgus monkeys. Animals were previously (3-4 weeks before study start) instrumented with indwelling pressure telemetry devices and trained to be restrained in chairs. Animals had access to food and drinking water ad libitum.

Test items were dissolved in water (Compound 1) or saline (0.9% NaCl; terlipressin) at a maximum stock concentration of 40 mg/mL and stored at −15° C. Stock solutions were diluted in saline on the day of administration. Animals with indwelling pressure telemetry devices were trained to be restrained in chairs for 8-h periods.

On the day of administration, animals were restrained, and telemetric measurements were collected continuously starting ≥2 h before administration. Baseline values of arterial pressure (diastolic and systolic), heart rate, and body temperature were obtained. After bolus administration (SC for Compound 1, IV for terlipressin), telemetric measurements were collected over 8 h after dosing.

The data reported herein were collected over 19 dosing sessions of four animals each, using a total of six monkeys. Animals were repeat dosed in a crossover design with at least one animal receiving vehicle for each round; the animal receiving vehicle was varied between rounds. A 48- to 72-h washout period was used between dosing sessions. Animals were observed by camera for ˜1 h before dosing (covering the baseline period) and up to 3 h after dosing.

In some instances, the data provided herein demonstrates that a compound described herein (e.g., Compound 1) is suitable for subcutaneous (e.g., bolus) administration in an individual (e.g., a mammal), such as at various doses in rats and monkeys. In some instances, the data provided herein demonstrates that a compound described herein (e.g., Compound 1) is suitable for intravenous (e.g., bolus) administration in an individual (e.g., a mammal), such as at various doses. In some instances, the data provided herein demonstrates that a compound described herein (e.g., Compound 1) is well-tolerated (e.g., when administered subcutaneously) in an individual (e.g., a mammal), such as at various doses in rats and monkeys. In some instances, the data provided herein also demonstrates that a compound described herein (e.g., Compound 1) increases MAP in an individual (e.g., a mammal), such increase being sustained over time (e.g., after subcutaneous administration). In some instances, the data provided herein also demonstrates that a compound described herein (e.g., Compound 1) decreases heart rate in an individual (e.g., a mammal), such as after subcutaneous administration. In some instances, changes in systolic blood pressure (SBP) and diastolic blood pressure (DBP) mirror overall changes in MAP. In some instances, the data provided herein also demonstrates that a compound described herein (e.g., Compound 1) has a differentiated pharmacologic profile in healthy individual (e.g., mammals that is consistent with MAP increases not being driven by differential changes in SBP versus DBP).

### Results

Pharmacokinetic Characterization of Compound 1 in Healthy Rats after IV and SC Administration

Plasma concentration-time profiles are shown in FIG. 22A and FIG. 22B. For IV administration, mean plasma concentration decreased in a mostly linear manner from ˜800 ng/mL to 2 ng/mL after 120 min. For SC administration, the mean (SD) plasma concentration normalized to a dose of 1.0 mg/kg (n=4). The mean normalized plasma concentration of Compound 1 was initially around 100 ng/mL, increased to approximately 300 ng/mL after 30 min and slowly decreased to approximately 50 ng/mL after 300 min.

PK parameters of Compound 1 after IV and SC bolus administration are shown in Table 17. The average values for V_{c}, Vₛₛ, t_{1/2}, and CL from four animals at a nominal dose of 0.10 mg/kg were 104 mL/kg, 164 mL/kg, 19 min, and 10 mL/min/kg for those with IV administration. The following parameters resulted from SC administration of Compound 1 (n=4, nominal dose of 1.0 mg/kg): Tₘₐₓ 35 min; t_{1/2Term} 113 min; AUC_{∞}/unit dose 59,965 min-ng/mL per mg/kg; CL/F 19 mL/min/kg; and F_{sc} 59%.

Compound 1 was well tolerated in adult male rats following single-dose IV bolus administration (cassette dosing). It was also well tolerated via the SC bolus administration via parallel individual administration.

TABLE 17

| | |
|---|---|
| Pharmacokinetic parameter | Mean ± SD |

Intravenous bolus

| | | |
|---|---|---|
| V_{c}, | mL/kg | 104 ± 29 |
| V_{SS}, | mL/kg | 164 ± 21 |
| t_{1/2Elim}, | min | 19 ± 3 |
| CL, | mL/min/kg | 10 ± 1 |

Subcutaneous bolus

| | |
|---|---|
| Cₘₐₓ per unit dose, ng/mL per mg/kg | 345 ± 116 |

| | | |
|---|---|---|
| Tₘₐₓ, | min | 35 ± 19 |
| t_{1/2Term}, | min | 113 ± 31 |

| | |
|---|---|
| AUC_{∞} per unit dose, min · ng/mL per mg/kg | 59,965 ± 29,227 |

| | | |
|---|---|---|
| CL/F, | mL/min/kg | 19 ± 7 |

| | |
|---|---|
| F_{SC}, % | 59 ± 29 |

CL, clearance; CL/F, apparent total body clearance; F_{SC}, fraction of the dosed substance reaching system circulation following administration by subcutaneous dose route (SC bioavailability); t_{1/2Elim}, elimination half-life; t_{1/2Term}, terminal half-life; V_{c}, initial apparent volume of the central compartment; V_{SS}, volume of distribution at steady state.

Compound 1 was designed as a selective V1a partial agonist using an agonist and an antagonist moiety in a single molecule to achieve effective partial agonism, demonstrating a differentiated pharmacologic profile. In some instances, Compound 1 binds to the V1a receptor in either its agonist or antagonist orientation, with each molecule binding only one receptor at a time. In some instances, binding of the agonist domain to V1a receptors drives the desired vasoconstrictive effect. In some instances, binding of the antagonist domain in a competitive manner prevents maximal activation of the V1a receptor pool. Cell-based functional assays of Compound 1 provide support for this partial agonism, the compound having no activity at the human or rat V2 receptor (as described elsewhere herein). This mechanism of action is further supported by the results of the pharmacokinetic and pharmacodynamic studies described herein. For example, the observed terminal half life (t_{1/2Term}) of Compound 1 after SC Administration (113 min) in rats was greater than the IV elimination half-life (t_{1/2Elim}; 19 min in rats), and the bioavailability after SC dosing was 59%. Also, the clearance of Compound 1 was similar to the glomerular filtration rate (GFR; 5-15 mL/min/kg) in male Sprague Dawley rats. Moreover, vasopressin showed potent full (V1a) agonism, whereas Compound 1 acted as a potent and partial V1a agonist in the SBF assay (FIG. 23A-C).

Pharmacodynamic Characterization of Compound 1 in Healthy Rats after IV and SC Administration

### Skin Blood Flow

Results for IV administration is shown in FIG. 23A-C. SBF showed dose-related decreases after IV administration of Compound 1 and AVP (FIG. 23A). Eₘₐₓ (95% CI) for Compound 1 and AVP were 38.6% (34.6-42.7) and 91.8% (85.5-98.0), respectively. ED₅₀ (95% CI) for Compound 1 and AVP were 25.2 pmoL/kg/min (15.1-35.4) and 12.6 pmoL/kg/min (9.18-16.0), respectively. The greatest percent change (SEM) from baseline in skin flow was −42.1% (3.9%) and −89.5% (1.9%) for Compound 1 (n=11) and AVP (n=13), respectively.

These data show that both Compound 1 and AVP are effective in reducing the SBF in anesthetized rats; however, AVP was a potent full agonist, whereas Compound 1 acted as a potent and partial agonist, with an approximately maximal decrease of 40% from baseline.

### Blood Lactate

Dose-response of blood lactate concentrations is shown in FIG. 23B. Mean (SEM) blood lactate concentrations did not increase after Compound 1 (n=11) administration, with doses ranging from 10 pmoL/kg/min to 3000 pmoL/kg/min. Mean (SEM) blood lactate concentrations increased after AVP administration (n=13), starting from 1.34 mM (0.18) at 3 pmoL/kg/min to 4.27 mM (0.39) at 600 pmoL/kg/min.

At the highest doses tested, blood lactate concentrations after AVP administration were significantly elevated compared with Compound 1 and vehicle (p<0.05; FIG. 23C). As increases in serum lactate levels are a clinical marker for anaerobic metabolism and tissue hypoxia, they can be used as a surrogate marker for the development of severe vasoconstriction and tissue ischemia. In this study, Compound 1 had no effect on blood lactate levels, regardless of dose up to the maximum dose tested, while AVP (vasopressin) significantly elevated blood lactate levels.

### Arterial Blood Pressure

Results for SC administration was tested at four doses (0.1, 0.6, 4.0, and 20 mg/kg; (FIG. 24A-D). Compared with pre-test values, there was a statistically significant increase in arterial blood pressure values (mean, systolic, and diastolic [MBP/SBP/DBP] components) in all the SC Compound 1-administered groups up to 4 h (at the 0.1 mg/kg dose), up to 6 h (at the 0.6 and 4.0 mg/kg doses), and up to 24 h (20 mg/kg; FIG. 24A-D). For each component of arterial blood pressure, the amplitude of increase was similar. The time of occurrence and amplitude of maximum increase in blood pressure were both dependent on the dose of Compound 1. Specifically, compared with the respective mean pre-test values for the mean arterial blood pressure component, the maximum increase was +22 mmHg at 1 and 2 h after administration of 0.1 and 0.6 mg/kg respectively; +38 mmHg at 3 and 4 h after a dose of 4 mg/kg; and +46 mmHg at 4 h after the 20 mg/kg dose. At the 24-h time point, there was still a ˜18% elevation of all blood pressure components. For the highest dose level (20 mg/kg), arterial blood pressure values did not return to pre-test values by the end of the evaluation period.

### Heart Rate

A slight decrease in heart rate was observed following SC administration of Compound 1 at 3 and 4 h with the 4.0 (291 and 278 bpm, respectively) and 20 (306 and 285 bpm, respectively) mg/kg dose levels compared with the control group (338 and 326 bpm; FIG. 24D).

### Safety and Tolerability

For safety considerations, no deaths occurred following SC administration in the control or Compound 1 administered animals. There was a local reaction at the injection site in three of the six males administered with dose levels of 4 and 20 mg/kg of Compound 1. The reaction was described as redness, skin irritation, erythema, or scab. Four administrations with different dose levels of Compound 1, up to 20 mg/kg, did not influence bodyweight gain.

Pharmacokinetic Characterization of Compound 1 in Healthy Monkeys after IV and SC Administration

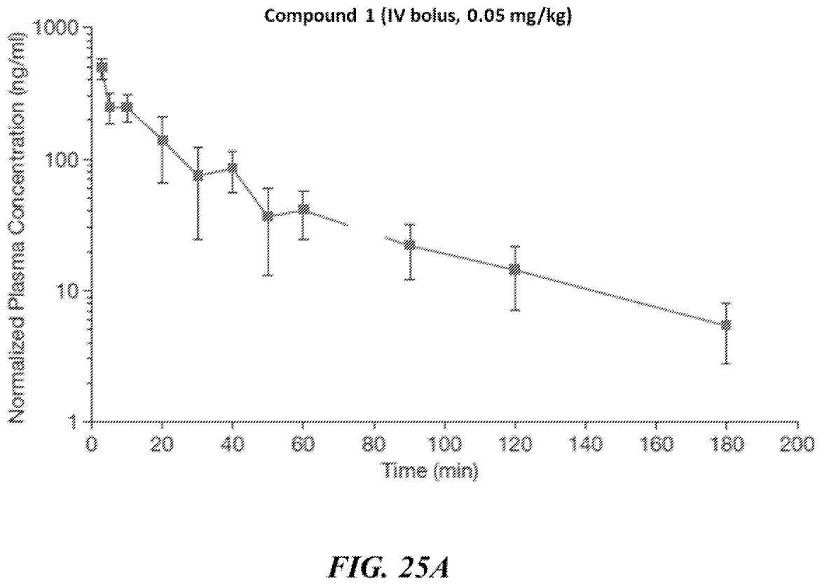

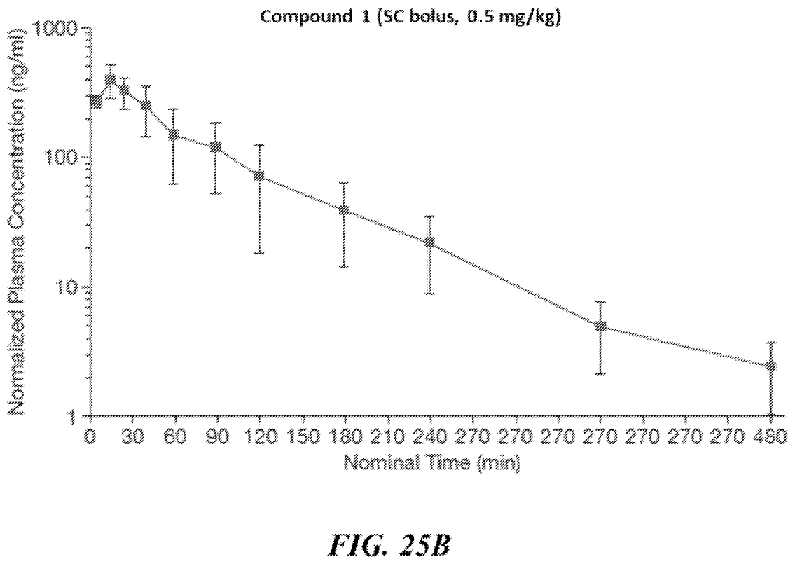

Following IV administration, the mean (SD) plasma concentration normalized to a dose of 0.05 mg/kg (FIG. 25A). The decrease was mostly linear from ˜500 to 5 ng/mL after 180 min (n=3). For SC administration, the plasma concentration normalized to a dose of 0.50 mg/kg (n=3). As shown in FIG. 25B, the mean normalized plasma concentration of Compound 1 was initially ˜300 ng/mL, increased to ˜400 ng/mL after 15 min, and slowly decreased to ˜3 ng/mL after 480 min.

The PK parameters of Compound 1 after IV/SC bolus administration are shown in Table 18. For IV administration at a nominal dose of 0.05 mg/kg (n=4), V_{c} was 45 mL/kg, Vₛₛ was 153 mL/kg, t_{1/2Elim} was 44 min, and CL was 4.6 mL/min/kg. For the three animals at a nominal dose of 0.5 mg/kg, the respective PK parameters from SC injection are as follows for Cₘₐₓ, Tₘₐₓ, t_{1/2Term}, AUC_{∞}/unit dose, CL/F, and F_{sc}: 801 ng/mL per mg/kg; 12 min; 70 min; 60,864 min-ng/mL per mg/kg; 19 mL/min/kg; and 30%.

TABLE 18

| | |
|---|---|
| Pharmacokinetic parameter | Mean ± SD |

Intravenous bolus

| | | |
|---|---|---|
| V_{c}, | mL/kg | 45 ± 23 |
| V_{SS}, | mL/kg | 153 ± 42 |
| t_{1/2Elim}, | min | 44 ± 5 |
| CL, | mL/min/kg | 4.6 ± 1.8 |

Subcutaneous bolus

| | |
|---|---|
| Cₘₐₓ per unit dose, ng/mL per mg/kg | 801 ± 220 |

| | | |
|---|---|---|
| Tₘₐₓ, | min | 12 ± 6 |
| t_{1/2Term}, | min | 70 ± 4 |

| | |
|---|---|
| AUC_{∞} per unit dose, min · ng/mL per mg/kg | 60,864 ± 25,980 |

| | | |
|---|---|---|
| CL/F, | mL/min/kg | 19 ± 11 |

| | |
|---|---|
| F_{SC}, % | 30 ± 21 |

CL, clearance; CL/F, apparent total body clearance; F_{SC}, fraction of the dosed substance reaching system circulation following administration by subcutaneous dose route (SC bioavailability); t_{1/2Elim}, elimination half-life; t_{1/2Term}, terminal half-life; V_{c}, initial apparent volume of the central compartment; V_{SS}, volume of distribution at steady state.

The pharmacokinetic and pharmacodynamic data provided herein for non-human primates are supportive of potent and partial V1a agonism of Compound 1. The clearance of Compound 1 was similar to the GFR (2-4 mL/min/kg) in cynomolgus monkeys after IV administration. The observed t_{1/2Term} of Compound 1 after SC administration (70 min) was greater than the IV t_{1/2Elim} (44 min) in monkeys, and the bioavailability after SC dosing was 30%.

### Pharmacodynamic Characterization of Compound 1 (SC) in Healthy Monkeys

In these animals, blood lactate concentrations were variable and not notably increased at 15, 40, 90, 180, or 360 min post dose relative to concentrations measured prior to Compound 1 SC administration. The pre-dose average blood lactate concentration was 2.8 mM (+/−0.9) and from 15 to 360 min ranged from 2.0 (±−1.0) to 3.9 (±−0.4) mM.

Results for SC administration of Compound 1 is illustrated in FIG. 26A-B. Compound 1 showed an increase from baseline in MAP (FIG. 26A), with the maximum, on average, similar across administration groups and occurring at 5-15 min after administration. For 0.01, 0.05, and 0.5 mg/kg, the respective maximum Δ MAP values were 36.1±2.1, 34.9±1.1, and 41.6±6.5 mmHg. Compared with vehicle-administered animals (vehicle SC: maximum Δ MAP=25.9±1.6 mmHg at 5 min after dosing), A MAP in Compound 1-administered animals was marked and long-lasting. The increases from baseline that were above 20 mmHg were sustained for an average of 150, 75, and 300 min after administration in the 0.01, 0.05, and 0.5 mg/kg administration groups, respectively.

A single SC bolus administration of Compound 1 at doses between 0.01 and 0.5 mg/kg caused large (e.g., significant) increases in arterial pressure in these non-human primates. Similar maximal increases from baseline in MAP, on average, across Compound 1 administration groups. In contrast, a single IV bolus administration of terlipressin at doses between 0.03 and 0.34 mg/kg caused strong but transient effects on MAP.

Between 30 min and 5 h after administration, the average increase in MAP were as follows for 0.01, 0.05, and 0.5 mg/kg groups: 20.6, 18.4, and 24.8 mmHg, respectively. For comparison, average increase in MAP was only 0.8 mmHg in the vehicle (SC)-administered animals. Markedly, Δ MAP was maintained above 14 mmHg, on average, throughout the 8 h of measurement in the 0.5 mg/kg Compound 1 administration group.

Compared to vehicle-administered animals, Δ MAP was statistically different from 5 to 420 min after administration in the 0.5 mg/kg Compound 1 group. For the 0.01 and 0.05 mg/kg Compound 1 groups, Δ MAP was statistically different versus vehicle-administered animals from 15 to 240 min and 15 to 210 min after administration, respectively.

Terlipressin induced an increase from baseline in MAP with the maximum, on average, occurring at 5-30 min after administration across administration groups (FIG. 26B). The following were maximum MAP values for doses 0.03, 0.09, 0.17, and 0.34 mg/kg: 29.6±3.6, 29.9±6.2, 50.8±5.6, and 41.2±7.5 mmHg, respectively. IV vehicle-administered animals showed a maximum Δ MAP of 22.2±3.5 mmHg at 5 min after dosing. Compared to vehicle-administered animals, MAP increases from baseline above 20 mmHg, on average, were not sustained past 30, 75, or 90 min after administration in the 0.03, 0.09, 0.17, and 0.34 mg/kg administration groups, respectively. Between 30 min and 5 h, the average increase in MAP was 6.0, 5.8, 14.6, and 14.7 mmHg as the dose increased in administration groups, respectively; vehicle-administered animals increased by 3.5 mmHg. The AMAP was negligible, on average, by 150 min (2.5 h) after administration in all terlipressin administration groups.

In non-human primates, the increases in arterial pressure after Compound 1 administration is sustained over time and accompanied by measurable decreases in heart rate. Similar maximal increases from baseline in MAP were observed across Compound 1 administration groups spanning a 50-fold dose range, which, in some instances, may represent the maximal effect on arterial pressure for this V1a receptor partial agonist. In contrast, the doses of terlipressin administered in this study caused strong but transient effects on MAP, with maximal increases from baseline in MAP occurring at 5-30 min across administration groups that were negligible by 150 min (2.5 h) after administration.

For each animal and dosing session, the pre-dose values of systolic, diastolic, and mean arterial pressures, heart rate, and body temperature were within the normal range of values generally observed per ERBC validation data. When video recordings from each dosing session were reviewed, no clinical signs were observed upon administration or during the 3 h after administration.

### Tolerability of Compound 1 in Healthy Individuals

While several animals experienced injection site reactions after subcutaneous (bolus) injection of Compound 1, in some instances, a single SC administration of Compound 1 to conscious rats was well tolerated at all tested dose levels. Study animals showed a slight diminution in heart rate at 3 and 4 h following SC administration of Compound 1, at dose levels of 4 and 20 mg/kg. Bradycardia is a potential reaction to a peripheral vasoconstriction indicates that the change in heart rate is administration related. However, this decrease in heart rate was not associated with any adverse events and, at this level, would not likely be clinically significant. Compound 1 administration also induced a dose-related increase in arterial blood pressure <24 h following the administration of the highest dose tested, which was clinically insignificant. In non-human primates, no apparent Compound 1-related effect on body temperature was noted, and Compound 1 was well tolerated following single-dose SC and IV bolus administrations.

### Example 6: Treating End-Stage Liver Disease (HRS-AKI) with Subcutaneous Administration of Mixed V1AR Agonist-Antagonist

The results described below demonstrate that a mixed V1a agonist/antagonists described herein (e.g., Compound 1) can be used to modulate mean arterial pressure (MAP) after subcutaneous administration.

In some instances, Compound 1 provides a submaximal effect on vasoconstriction, such as highlighting the dual benefits of therapeutic effectiveness and improved safety through less risk of excessive vasoconstriction. In some instances, administration of Compound 1 produced desired beneficial effects on factors relating to kidney sodium and water retention without apparent unwanted effects or nonspecific toxicities.

### Study 1—Bile Duct Ligation (BDL) Rat Model

In some instances, bile duct ligation (BDL) induces cholestatic cirrhosis in rats and in other mammals (e.g., monkeys). Following double ligation and excision of the extrapancreatic common bile duct, biliary cirrhosis develops over several weeks. In addition to hemodynamic changes, such as PHT and decreased splanchnic vascular resistance, BDL rats develop ascites and renal impairment. In some embodiments, the BDL rat model described herein is a model of decompensated cirrhosis.

### Materials:

Animals: Male Sprague Dawley rats weighing 300-400 grams with bile duct ligations (BDL) performed at 7 weeks of age by the vendor (Harlan, IN). BDL rats were administered 50 μg of vitamin K1 at the time of BDL surgery, prior to shipment, and then once weekly to reduce potential mortality from hemorrhagic complications. BDL surgery included 4 ligations and 1 cut: two ligatures were placed around duct branches from the middle lobe, a third ligature was placed around the duct branch from the left lateral lobe, a fourth ligature was placed around the common bile duct caudal to the three previously ligated branches, and a cut into the common bile duct was made immediately cranial to the forth ligature and caudal to the initial middle lobe ligature.

Compound formulation and administration: Compound 1 was formulated as a stock solution (15 mg powder/mL) in water, and stored at 4° C. A working solution was prepared by dilution of the stock solution with saline (100 μg powder/mL, 99.5 μg FB/mL). The working solution was stored at 4° C. over the course of the study and warmed to room temperature for 30 minutes before administration. Compound 1 was administered in a dose volume of 1 mL/kg subcutaneously by injection into the animal's lower back. Animals received Compound 1 or vehicle, twice a day, over 5 days; the final administration was on the morning of the fifth day, for a total of 9 administrations; animals were sacrificed before the second administration on day 5.

### Experimental Protocol

Briefly, rats had common bile duct ligations for 5 weeks before compound administration, at which time ascites was apparent and cirrhosis should have been established. Animals were divided into two treatment groups receiving Compound 1 (99.5 μg FB/kg, N=8) or vehicle (0.9% saline, N=9), subcutaneously twice a day over 5 days; the final treatment was on the morning of the fifth day, for a total of 9 administrations.

Animals were weighed daily. After the first treatment administration animals were placed into metabolic cages for 24 hours for urine collection and measurement of water/food intake. Each metabolic cage was set up for continuous measurement of spontaneous urine output via force transducers placed above the urine collection vials to monitor and record the time course of urine output. Data from the calibrated force transducers every time the rats void. Urine production was monitored continuously in the first 24 hours. Urine was collected at two time points, at 4 hours post the first administration (4 hours of urine), and from 5 to 24 hours post the first administration (20 hours of urine). Food and water intake were recorded at 24 hours post the first administration. Only 16 metabolic cages could be used, urine, water, and food intake data were not collected for one animal in the vehicle group. Animals were returned to their home cages for the remainder of the study and were sacrificed before the second treatment on the fifth day. Blood samples were collected from each animal via cardio puncture. Laparotomies were performed and ascites fluid collected and volume measured by pipet. Spleens were dissected from each animal, any excessive fat or connective tissue was removed and the spleens were weighted.

Collected parameters for final reporting were:

Body weight (g): on each day of treatment administration (days 1-5)

Water intake (mL): water intake measured over 24 hours following the first treatment administration on day 1

Spleen weight (g): spleen weight measured after animal sacrifice on day 5 following 9 treatment administrations, b.i.d.

### Results

In some instances, a compound described herein (e.g., Compound 1) modulates pathophysiology in a BDL rat model. In some instances, BDL is used to block enterohepatic recirculation of bile acids, which can provide severe hepatic injury, collagen accumulation in the hepatic parenchyma, and excessive elevation in PP (14-20 mmHg).

Following administration of Compound 1, the observed PP decreased from baseline. The observed PP values were not significantly different between any of the four treatment groups based on MANOVA with repeated measures. Across all treatment groups, dose-related decreases from baseline in portal pressure (APP) occurred following Compound 1 administration, with a plateau as the dose increased further (Table 10, FIG. 16). In the Compound 110, 25, 100, and 500 μg/kg treatment groups, the maximum ΔPP (mean, SEM) were −1.8±0.2 mmHg at 7 min, −3.3±0.2 mmHg at 10 min, −4.4±0.2 mmHg at 3 min, and −4.9±0.6 mmHg at 7 min, respectively. The change in PP remained negative through the testing period (at 30 m) in all treatment groups: −1.2±0.2, −2.9±0.5, 3.4±0.2, and −3.6±0.7 mmHg, respectively. When comparing results for ΔPP, the observed PP values at baseline (time 0) were statistically different between the 10 μg/kg (16.9±0.7 mmHg) and 500 μg/kg (20.1±0.7 mmHg) Compound 1 treatment groups. This was determined by one-way ANOVA with Tukey-Kramer HSD post hoc analysis (p=0.45).

TABLE 10

| | Δ PP (mmHg) | | | | | | | | | | | |
|---|---|---|---|---|---|---|---|---|---|---|---|---|
| Time | 10 μg/kg, SC | | | 25 μg/kg, SC | | | 100 μg/kg, SC | | | 500 μg/kg, SC | | |
| (min) | Mean | SEM | N | Mean | SEM | N | Mean | SEM | N | Mean | SEM | N |
| 0 | 0.0 | 0.0 | 9 | 0.0 | 0.0 | 7 | 0.0 | 0.0 | 14 | 0.0 | 0.0 | 8 |
| 1 | −0.2 | 0.1 | 9 | −0.6 | 0.3 | 7 | −2.1 | 0.3 | 14 | −3.5 | 0.5 | 8 |
| 2 | −0.7 | 0.1 | 9 | −1.7 | 0.4 | 7 | −4.2 | 0.3 | 14 | −4.4 | 0.5 | 8 |
| 3 | −1.2 | 0.2 | 9 | −2.8 | 0.3 | 7 | −4.4 | 0.2 | 14 | −4.6 | 0.5 | 8 |
| 5 | −1.7 | 0.2 | 9 | −3.1 | 0.2 | 7 | −4.1 | 0.2 | 14 | −4.8 | 0.5 | 8 |
| 7 | −1.8 | 0.2 | 9 | −3.2 | 0.2 | 7 | −4.1 | 0.2 | 14 | −4.9 | 0.6 | 8 |
| 10 | −1.7 | 0.2 | 9 | −3.3 | 0.2 | 7 | −4.0 | 0.2 | 14 | −4.8 | 0.6 | 8 |
| 15 | −1.7 | 0.2 | 9 | −3.3 | 0.3 | 7 | −3.6 | 0.2 | 13 | −3.8 | 0.7 | 8 |
| 20 | −1.5 | 0.2 | 9 | −3.1 | 0.4 | 7 | −3.5 | 0.2 | 13 | −3.8 | 0.6 | 8 |
| 30 | −1.2 | 0.2 | 9 | −2.9 | 0.5 | 7 | −3.4 | 0.2 | 13 | −3.6 | 0.7 | 7 |

PP, portal pressure;
SC, subcutaneous

### Study 2—Methionine/Choline-Deficient (MCD) Diet Rat Model of Cirrhosis and Portal Hypertension

In some instances, a diet low in choline and methionine (i.e., methionine/choline-deficient (MCD) diet) is used as an established rat model to induce cirrhosis and its complications, such as PHT and elevated PP.

In some instances, the MCD diet rat model described herein is an ESLD model for treating liver damage and complications associated therewith, such as ascites.

### Materials

Animals: Thirty adult male Wistar rats (Harlan, Indianapolis) were studied over multiple testing days and weighed 200-250 g at the start of the study. Animals were housed two per cage in a controlled environment with free access to an MCD diet and water.

Compositions and doses: A compound provided herein was administered in a dose volume of 1 mL/kg subcutaneously by injection into the animal's lower back. Terlipressin was administered intra-arterially via the femoral catheter used to measure pressure. Doses of the compounds provided herein were 500, 100, 25, and 10 μg/kg. The terlipressin dose was 100 μg/kg.

### Experimental Protocol

Administration: On the day of experimental measurements for each study, animals were anesthetized and catheters were flushed to maintain or restore patency. Catheters were then connected to fluidic pressure transducers linked to data acquisition stations. Prior to administration of compound, a stable baseline was obtained for the pressure readings, saline was administered, and an additional 5-15 min of pressure readings were taken to solidify the baseline readings and provide a per-animal vehicle control. After ≥8 weeks on the MCD diet, the adult male Wistar rats underwent surgical placement of catheters in the portal vein and/or femoral artery. Animals were allowed to recover for ≥5 days. Compounds were bolus administered as a subcutaneous injection for Compound 1 (500, 100, and 25 μg/kg) or intra-arterially for terlipressin (100 μg/kg). Systolic blood pressure (SBP), diastolic blood pressure (DBP), and PP were measured by the fluidic transducers. Measurements were recorded continuously from time 0 (compound administration time) to 90 min following administration, unless technical difficulties (such as loss of catheter patency) resulted in cessation of the experiment prior to 90 min. Thirty animals had repeat dosing with >1 day of recovery between administrations. Altogether, 86 measurements with 1-8 repeat administrations per animal were used.

Data analysis: Data recorded by NOTOCORD-hem™ were transferred to Microsoft Excel for analysis. Data were evaluated as change (Δ) from baseline. MAP and PP were reported as the average pressure value (mmHg) recorded over 10 s beginning at the first systole (PP and SBP) or diastole (DBP) following the time point of interest at nominal times of 0 (compound administration), 1, 2, 3, 5, 7, 10, 15, 20, and 30 min after administration in both studies, along with 60 and 90 min after administration in the MCD study. Data collected over multiple test days were compiled. Mean, SEM, and N were reported for PP, MAP, ΔPP, and ΔMAP for each compound dose and time point.

MAP was calculated from SBP and DBP: MAP=[(2×DBP)+SBP]/3.

Change from baseline (delta A) for PP and MAP at each time point in each animal were calculated as follows: ΔPP=PP at each time −PP at baseline (time 0) and ΔMAP=MAP at each time −MAP at baseline (time 0).

Statistical Analysis: The observed PP and MAP data were statistically analyzed using JMP software. Data from each animal at times 0 to 90 min (MCD study) and 0 to 30 min (BDL study) in the four treatment groups were compared using MANOVA with repeated measures. Each time course measurement was considered independent; animals were allowed to recover before any repeat administration. No corrections or extrapolations were performed if data were not collected for every time point. The MANOVA outcome for treatment between subjects was not considered significant if p≥0.05 (Prob>F); in the MCD study, subsequent contrasts were performed to compare treatment groups. Data from time 0 were statistically determined using one-way ANOVA to compare starting values in each treatment group. In cases when this ANOVA was significant (p<0.05), Tukey-Kramer HSD post hoc analysis was used to compare treatment groups.

### Results

In some instances, a compound described herein (e.g., Compound 1) modulates disease physiology in an MCD model of PHT.

In some instances, the effects of Compound 1 and terlipressin on MAP were examined using rats on an MCD diet. The diet was given for 9-19 weeks before testing. Both Compound 1 and terlipressin resulted in increased MAP. For the 25, 100, and 500 μg/kg Compound 1 treatment groups, the highest observed absolute MAP values (mean±SEM) were 79.2±1.4, 86.8±2.5, and 90.4±3.7 mmHg, respectively. There were greater increases in MAP resulting from the intra-arterial administration of terlipressin (100 μg/kg) compared with the Compound 1 treatment groups, with the highest observed MAP of 116.6±4.1 mmHg.

In some instances, Compound 1 administered at different doses provided a significant difference in effect. For example, observed MAP using MANOVA with repeated measures of the Compound 1 treatment groups showed a significant difference between the 25 and 100 μg/kg Compound 1 treatment groups (p=0.03). Further, there was no significant difference between the 100 and 500 μg/kg Compound 1 treatment groups (p=0.63). Additionally, MAP was significantly different between intra-arterial administration of terlipressin and subcutaneous administration of Compound 1 at 500 μg/kg (p<0.01).

In the study, the maximum mean change in mean arterial pressure (ΔMAP) values (mean C SEM) for Compound 1 were 5.3±1.1, 11.3±1.8, and 14.7±2.6 mmHg in the 25, 100, and 500 μg/kg treatment groups, respectively (Table 11, FIG. 17). Following intra-arterial administration of terlipressin, the maximum ΔMAP (33.5±2.6 mmHg) was greater than in any of the Compound 1 treatment groups. FIG. 17 illustrates that Compound 1 has a relatively large therapeutic window (over a large dose range and over a prolonged period of time), such as for changing (increasing) MAP, relative to terlipressin. FIG. 17 illustrates that Compound 1 is useful for treating ESLD or symptoms and/or complications thereof, such as over a large dose range and over a prolonged period of time. FIG. 17 also illustrates that terlipressin can quickly reach toxic and potentially harmful concentrations, for example providing rapid increases in MAP (increases up to about 35 mmHg) that are above safe and/or therapeutic levels. FIG. 17 also illustrates that the effect of terlipressin can diminish rapidly, for example quickly falling below therapeutic levels after a relatively short period of time (e.g., after about 80 mins or more).

TABLE 11

| | Δ MAP (mmHg) | | | | | | | | | | | |
|---|---|---|---|---|---|---|---|---|---|---|---|---|
| | Compound 1 | | | | | | | | | Terlipressin | | |
| Time | 25 μg/kg, SC | | | 100 μg/kg, SC | | | 500 μg/kg, SC | | | 100 μg/kg, IA | | |
| (min) | Mean | SEM | N | Mean | SEM | N | Mean | SEM | N | Mean | SEM | N |
| 0 | 0.0 | 0.0 | 13 | 0.0 | 0.0 | 23 | 0.0 | 0.0 | 17 | 0.0 | 0.0 | 13 |
| 1 | 1.1 | 0.2 | 13 | 2.0 | 0.5 | 22 | 2.2 | 0.5 | 17 | 6.1 | 0.8 | 13 |
| 2 | 1.9 | 0.5 | 13 | 3.5 | 0.7 | 22 | 4.5 | 0.7 | 17 | 10.8 | 1.2 | 13 |
| 3 | 2.6 | 0.6 | 13 | 4.4 | 0.7 | 22 | 6.4 | 0.9 | 17 | 18.1 | 2.1 | 13 |
| 5 | 3.5 | 0.7 | 13 | 7.0 | 1.2 | 22 | 9.1 | 1.4 | 17 | 26.3 | 2.8 | 13 |
| 7 | 4.0 | 0.9 | 12 | 8.5 | 1.5 | 21 | 12.1 | 1.8 | 17 | 30.1 | 2.9 | 13 |
| 10 | 4.8 | 1.0 | 13 | 9.9 | 1.4 | 22 | 14.7 | 2.6 | 17 | 32.0 | 2.5 | 13 |
| 15 | 5.3 | 1.1 | 13 | 11.0 | 1.6 | 23 | 13.6 | 2.8 | 17 | 32.9 | 2.7 | 13 |
| 20 | 5.2 | 1.3 | 13 | 11.3 | 1.8 | 23 | 13.7 | 3.2 | 16 | 33.5 | 2.6 | 13 |
| 30 | 4.4 | 1.5 | 13 | 10.6 | 2.2 | 20 | 11.4 | 3.0 | 17 | 31.7 | 2.5 | 13 |
| 60 | 3.0 | 1.7 | 13 | 10.9 | 3.5 | 17 | 9.1 | 2.5 | 17 | 13.3 | 2.6 | 13 |
| 90 | 3.1 | 1.8 | 12 | 9.2 | 3.1 | 17 | 10.2 | 3.0 | 16 | 5.2 | 2.1 | 13 |

IA, intra-arterial;
MAP, mean arterial pressure;
SC, subcutaneous

Additionally, the observed MAP baseline values (time 0) were statistically different for the terlipressin group versus each of the Compound 1 treatment groups. The values were determined by one-way ANOVA with Tukey-Kramer HSD post hoc analysis (p=0.02 vs 25 μg/kg, p=0.03 vs 100 μg/kg, p=0.46 vs 500 μg/kg Compound 1).

In some instances, Compound 1 provided a decrease in portal pressure (PP). For example, there was a decrease in PP following treatment with Compound 1 or terlipressin. The observed PP values were not significantly different between any of the four treatment groups, as determined by MANOVA with repeated measures. The maximum ΔPP (mean±SEM) were −2.3±0.2, −2.5±0.3, and −3.9±0.8 mmHg in the Compound 1 25, 100, and 500 g/kg treatment groups, respectively (Table 13, FIG. 18). The change following intra-arterial administration of terlipressin was −2.8±0.8 mmHg. The observed PP values at baseline (time 0) were not statistically different between the treatment groups, as determined by one-way ANOVA with Tukey-Kramer HSD post hoc analysis.

TABLE 13

| | Δ PP (mmHg) | | | | | | | | | | | |
|---|---|---|---|---|---|---|---|---|---|---|---|---|
| | Compound 1 | | | | | | | | | Terlipressin | | |
| Time | 25 μg/kg, SC | | | 100 μg/kg, SC | | | 500 μg/kg, SC | | | 100 μg/kg, IA | | |
| (min) | Mean | SEM | N | Mean | SEM | N | Mean | SEM | N | Mean | SEM | N |
| 0 | 0.0 | 0.0 | 19 | 0.0 | 0.0 | 24 | 0.0 | 0.0 | 13 | 0.0 | 0.0 | 6 |
| 1 | −0.3 | 0.1 | 19 | −0.5 | 0.2 | 24 | −1.2 | 0.2 | 13 | −1.5 | 0.4 | 6 |
| 2 | −0.8 | 0.2 | 19 | −1.2 | 0.3 | 24 | −1.9 | 0.4 | 13 | −2.1 | 0.4 | 6 |
| 3 | −1.3 | 0.3 | 19 | −1.6 | 0.4 | 24 | −2.5 | 0.5 | 13 | −2.3 | 0.4 | 6 |
| 5 | −1.8 | 0.4 | 19 | −2.1 | 0.4 | 24 | −2.9 | 0.6 | 13 | −2.4 | 0.4 | 6 |
| 7 | −1.9 | 0.3 | 19 | −2.5 | 0.3 | 23 | −3.3 | 0.8 | 13 | −2.5 | 0.4 | 6 |
| 10 | −2.1 | 0.3 | 19 | −2.5 | 0.3 | 24 | −2.9 | 0.7 | 13 | −2.4 | 0.5 | 6 |
| 15 | −2.2 | 0.3 | 19 | −2.4 | 0.3 | 23 | −3.5 | 0.8 | 12 | −2.5 | 0.6 | 6 |
| 20 | −2.3 | 0.2 | 19 | −2.3 | 0.3 | 23 | −3.8 | 0.7 | 12 | −2.6 | 0.6 | 6 |
| 30 | −2.3 | 0.3 | 18 | −2.2 | 0.2 | 20 | −3.9 | 0.8 | 12 | −2.5 | 0.8 | 6 |
| 60 | −2.2 | 0.3 | 16 | −2.0 | 0.2 | 17 | −3.4 | 0.8 | 11 | −2.8 | 0.8 | 6 |
| 90 | −1.8 | 0.3 | 14 | −1.7 | 0.2 | 16 | −3.7 | 1.0 | 8 | −0.8 | 0.3 | 5 |

IA, intra-arterial;
PP, portal pressure;
SC, subcutaneous

We claim:

1. A method of modulating mean arterial pressure (MAP) in an individual (e.g., in need thereof), the method comprising subcutaneously infusing into the individual (e.g., in need thereof) a composition comprising an (effective) amount of a compound, the compound being a mixed vasopressin receptor 1A (V1AR) agonist-antagonist.

2. The method of claim 1, wherein the mixed vasopressin receptor 1A (V1AR) agonist-antagonist is selective for V1AR over V2R.

3. The method according to claim 1 or 2, wherein the mixed vasopressin receptor 1A (V1AR) agonist-antagonist has no V2R activity, such as at therapeutic concentrations.

4. The method of any one of the preceding claims, wherein the compound comprises a first portion having agonist activity and a second portion having antagonist activity.

5. The method of any one of the preceding claims, wherein the modulation of mean arterial pressure (MAP) in the individual comprises raising MAP by at least 5% over baseline.

6. The method of any one of the preceding claims, wherein the modulation of mean arterial pressure (MAP) in the individual comprises raising MAP by at least 5 mmHg (e.g., 5 mmHg or more, or 10 mmHg or more) over baseline.

7. The method of any one of the preceding claims, wherein the compound has a structure represented by Formula I: 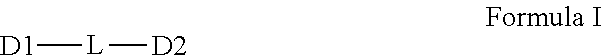
or a pharmaceutically acceptable salt thereof,
wherein:
D1 is a vasopressin receptor 1A (V1AR) agonist;
D2 is a V1AR antagonist; and
L is a linker.

8. A method of modulating mean arterial pressure (MAP) in an individual (e.g., in need thereof), the method comprising subcutaneously infusing into the individual (e.g., in need thereof) an (effective) amount of a compound having a structure represented by Formula I: 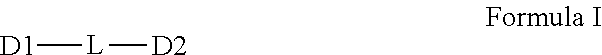
or a pharmaceutically acceptable salt thereof,
wherein:
D1 is a vasopressin receptor 1A (V1AR) agonist;
D2 is a V1AR antagonist; and
L is a linker.

9. The method of claim 8, wherein D1 is selective for V1AR over V2R.

10. The method according to claim 8 or 9, wherein D1 is or comprises a (e.g., cyclic) peptide.

11. The method of any one of claims 8-10, wherein D1 is or comprises a cyclic nonapeptide.

12. The method of any one of claims 8-11, wherein D1 has or comprises the following structure:

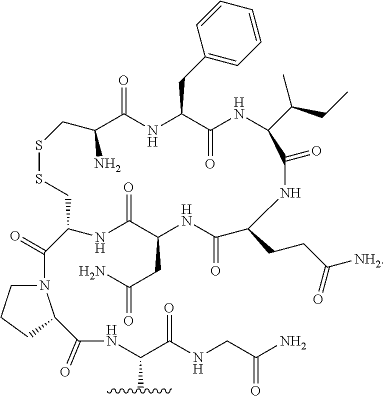 [SMILES: *[C@H](NC(=O)[C@@H]1CCCN1C(=O)[C@@H]1CSSC[C@H](N)C(=O)N[C@@H](Cc2ccccc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

13. The method of any one of claims 8-12, wherein D2 is or comprises a (e.g., linear) peptide.

14. The method of any one of claims 8-13, wherein D2 is a linear polypeptide comprising about seven or more amino acid residues.

15. The method of any one of claims 8-14, wherein D2 has or comprises the following structure:

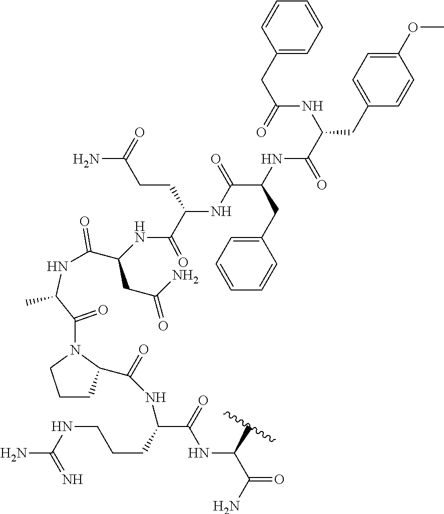 [SMILES: *[C@H](NC(=O)[C@H](CCCNC(=N)N)NC(=O)[C@@H]1CCCN1C(=O)[C@H](C)NC(=O)[C@H](CC(N)=O)NC(=O)[C@H](CCC(N)=O)NC(=O)[C@H](Cc1ccccc1)NC(=O)[C@@H](Cc1ccc(OC)cc1)NC(=O)Cc1ccccc1)C(N)=O]

16. The method of any one of claims 8-15, wherein L is a non-hydrolyzable linker.

17. The method of any one of claims 8-16, wherein L comprises one or more linker group, each linker group being independently selected from the group consisting of a substituted or unsubstituted alkyl and substituted or unsubstituted heteroalkyl.

18. The method of any one of claims 8-17, wherein L is a bond, substituted or unsubstituted alkyl, or substituted or unsubstituted heteroalkyl.

19. The method of any one of claims 8-18, wherein L is or comprises substituted or unsubstituted heteroalkyl.

20. The method of any one of claims 8-19, wherein L is heteroalkyl (e.g., alkylamine) substituted with one or more substituent, each substituent being independently selected form the group consisting of oxo, amino, and substituted heteroalkyl (e.g., alkylamine substituted with oxo).

21. The method of any one of claims 8-20, wherein L is or comprises one or more (e.g., modified) amino acid residue.

22. The method of any one of claims 8-21, wherein L has or comprises the following structure:

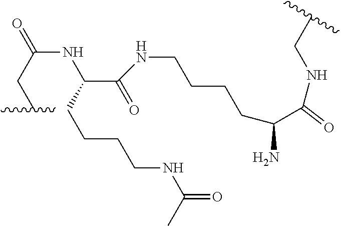 [SMILES: *CNC(=O)[C@@H](N)CCCCNC(=O)[C@H](CCCCNC(C)=O)NC(=O)C*]

23. The method of any one of claims 8-22, wherein the compound is Compound 1, or a pharmaceutically acceptable salt thereof.

24. A method of modulating mean arterial pressure (MAP) in an individual (e.g., in need thereof), the method comprising subcutaneously infusing into the individual (e.g., in need thereof) a composition comprising an (effective) amount of Compound 1, or a pharmaceutically acceptable salt thereof.

25. The method of any one of the preceding claims, wherein the composition further comprises a liquid vehicle or solvent (e.g., water or an aqueous vehicle).

26. The method of any one of the preceding claims, wherein the method further comprises affixing a subcutaneous infusion device to the skin of the individual, the subcutaneous infusion device comprising a chamber body and a hollow tube body, the composition being configured within the chamber body, the hollow tube body comprising a first opening and a second opening, the first opening being in fluid contact with the chamber body, and the second opening being configured subcutaneously within the individual after affixing the subcutaneous infusion device to the skin.

27. The method of claim 26, wherein the subcutaneous infusion device further comprises a pump configured to subcutaneously infuse the composition into the individual at a constant or varying rate.

28. A method of modulating mean arterial pressure (MAP) in an individual (e.g., in need thereof), the method comprising subcutaneously infusing into the individual (e.g., in need thereof) an effective amount of Compound 1, or a pharmaceutically acceptable salt thereof.

29. A method of modulating mean arterial pressure (MAP) in an individual (e.g., in need thereof), the method comprising subcutaneously administering to the individual (e.g., in need thereof) a composition comprising an (effective) amount of a compound, the compound being a mixed vasopressin receptor 1A (V1AR) agonist-antagonist.

30. A method of modulating mean arterial pressure (MAP) in an individual (e.g., in need thereof), the method comprising subcutaneously administering to the individual (e.g., in need thereof) a composition comprising an (effective) amount of a compound having a structure represented by Formula I: 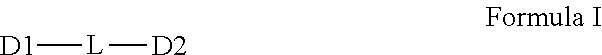
or a pharmaceutically acceptable salt thereof,
wherein:
D1 is a vasopressin receptor 1A (V1AR) agonist;
D2 is a V1AR antagonist; and
L is a linker.

31. A method of modulating mean arterial pressure (MAP) in an individual (e.g., in need thereof), the method comprising subcutaneously administering to the individual (e.g., in need thereof) a composition comprising an (effective) amount of Compound 1, or a pharmaceutically acceptable salt thereof.

32. The method of any one of the preceding claims, wherein when the composition is subcutaneously administered to the individual (e.g., by subcutaneous (bolus) injection or subcutaneous infusion), less than 50% of the compound of Formula I degrades (e.g., subcutaneously).

33. The method of any one of the preceding claims, wherein when the composition is subcutaneously administered to the individual (e.g., by subcutaneous (bolus) injection or subcutaneous infusion), less than 30% of the compound of Formula I degrades (e.g., subcutaneously).

34. The method of any one of the preceding claims, wherein when the composition is subcutaneously administered to the individual (e.g., by subcutaneous (bolus) injection or subcutaneous infusion), less than 50% of the compound of Formula I degrades (e.g., subcutaneously) to form M1.

35. The method of any one of the preceding claims, wherein when the composition is subcutaneously administered to the individual (e.g., by subcutaneous (bolus) injection or subcutaneous infusion), less than 30% of the compound of Formula I degrades (e.g., subcutaneously) to form M1.

36. The method of any one of the preceding claims, wherein when the composition is subcutaneously infused into the individual less M1 is formed relative to administration of an otherwise identical composition administered by subcutaneous (bolus) injection.

37. The method of any one of the preceding claims, wherein when the composition is subcutaneously infused into the individual less M1 is formed systemically relative to administration of an otherwise identical composition administered by subcutaneous (bolus) injection.

38. The method of any one of the preceding claims, wherein when the composition is subcutaneously infused into the individual less M1 is formed locally (injection/infusion site) relative to administration of an otherwise identical composition administered by subcutaneous (bolus) injection.

39. The method of any one of the preceding claims, wherein subcutaneously infusing the composition into the individual improves tolerability, relative to subcutaneous (bolus) injection (e.g., based on a reduction of M1 overproduction, such as subcutaneously).

40. The method of any one of the preceding claims, wherein subcutaneously infusing the composition into the individual reduces undesired systemic events (e.g., undesired vasoconstriction, such as resulting in ischemia), reduces undesired administration site events (e.g., local site vasoconstriction, such as resulting in administration site ischemia), or both.

41. The method of any one of the preceding claims, wherein the composition is subcutaneously infused into the individual continuously for at least one hour.

42. The method of any one of the preceding claims, wherein the composition is subcutaneously infused into the individual at a rate of about 0.005 milliliters per hour (mL/hr) to about 1 mL/hr for an administration period.

43. The method of any one of the preceding claims, wherein the composition comprises a buffering agent.

44. The method of claim 43, wherein the buffering agent is selected from the group consisting of acetate buffer, succinate buffer, and citrate buffer.

45. The method of any one of the preceding claims, wherein the composition comprises a buffering agent in a concentration of about 1 millimolar (mM) to about 1 molar (M).

46. The method of any one of the preceding claims, wherein the composition comprises a buffering agent in a concentration of about 5 mM to about 250 mM.

47. The method of any one of the preceding claims, wherein the composition comprises a buffering agent in a concentration of about 5 mM to about 25 mM.

48. The method of any one of claims 1-46, wherein the composition comprises a buffering agent in a concentration of about 50 mM to about 250 mM.

49. The method of any one of the preceding claims, wherein the composition has a pH of about 4 to about 8.

50. The method of any one of the preceding claims, wherein the composition has a pH of about 4 to about 6.

51. The method of any one of the preceding claims, wherein the composition has a pH of about 4.5 to about 5.

52. The method of any one of the preceding claims, wherein the compound is administered to the individual (e.g., continuously) in an amount of about 0.001 milligram (mg) to about 100 mg, such as over a period of one or more days.

53. The method of any one of the preceding claims, wherein the composition comprises the compound in a concentration of about 0.001 milligrams per milliliters (mg/mL) to about 100 mg/mL.

54. The method of any one of the preceding claims, wherein the composition comprises the compound in a concentration of about 0.1 mg/mL to about 100 mg/mL.

55. The method of any one of the preceding claims, wherein the composition comprises the compound in a concentration of about 1 mg/mL to about 10 mg/mL.

56. The method of any one of the preceding claims, wherein the composition further comprises a preservative.

57. The method of claim 56, wherein the preservative is present in an amount of about 1 mg/mL to about 20 mg/mL.

58. The method of any one of the preceding claims, wherein the composition further comprises a solubilizing agent.

59. The method of claim 58, wherein the solubilizing agent is present in an amount of about 1 mg/mL to about 250 mg/mL (e.g., about 60-80 mg/mL).

60. The method of any one of the preceding claims, the compound is (continuously) administered to the individual in need thereof at a dose of about 0.1 mg/day to about 100 mg/day.

61. A method of reducing (incidence of) local vasoconstriction, such as (injection site) ischemia, in an individual in need thereof, the method comprising subcutaneously infusing into the individual in need thereof a composition comprising a compound having a structure represented by Formula I, or a pharmaceutically acceptable salt thereof: 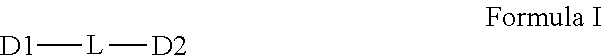
or a pharmaceutically acceptable salt thereof,
wherein:
D1 is a vasopressin receptor 1A (V1AR) agonist;
D2 is a V1AR antagonist; and
L is a linker.

62. A method of modulating mean arterial pressure (MAP) in an individual in need thereof, the method comprising subcutaneously infusing into the individual in need thereof a composition comprising a compound having a structure represented by Formula I, or a pharmaceutically acceptable salt thereof: 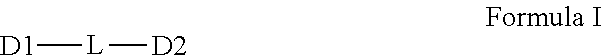
or a pharmaceutically acceptable salt thereof,
wherein:
D1 is a vasopressin receptor 1A (V1AR) agonist;
D2 is a V1AR antagonist; and
L is a linker,
wherein less than 50% of the compound of Formula I degrades (e.g., subcutaneously).

63. The method of any one of the preceding claims, wherein the MAP of the individual increases (e.g., compared to a baseline measurement before treatment) following subcutaneous administration of Compound 1.

64. The method of any one of the preceding claims, wherein the MAP of the individual increases by about 1% to about 10% (e.g., compared to a baseline measurement before treatment) after administering the compound, or the pharmaceutically acceptable salt thereof, of any one of the preceding claims to the individual.

65. The method of any one of the preceding claims, wherein the MAP of the individual dose-dependently increases after administering the compound, or the pharmaceutically acceptable salt thereof, of any one of the preceding claims to the individual.

66. The method of any one of the preceding claims, wherein diastolic blood pressure of the individual increases (e.g., compared to a baseline measurement before treatment) after administering the compound, or the pharmaceutically acceptable salt thereof, of any one of the preceding claims to the individual.

67. The method of any one of the preceding claims, wherein systolic blood pressure of the individual increases (e.g., compared to a baseline measurement before treatment) after administering the compound, or the pharmaceutically acceptable salt thereof, of any one of the preceding claims to the individual.

68. The method of any one of the preceding claims, wherein the diastolic and/or systolic blood pressure of the individual dose-dependently increases after administering the compound, or the pharmaceutically acceptable salt thereof, of any one of the preceding claims to the individual.

69. The method of any one of the preceding claims, wherein pulse rate and/or peripheral blood flow of the individual decreases after administering the compound, or the pharmaceutically acceptable salt thereof, of any one of the preceding claims to the individual.

70. The method of any one of the preceding claims, wherein (subcutaneous) administration of the compound, or the pharmaceutically acceptable salt thereof, of any one of the preceding claims to the individual improves systemic hemodynamics in the individual.

71. The method of any one of the preceding claims, wherein (subcutaneous) administration of the compound, or the pharmaceutically acceptable salt thereof, of any one of the preceding claims to the individual reduces fluid retention and/or overload in the individual.

72. The method of any one of the preceding claims, wherein the method comprises administering the compound, or the pharmaceutically acceptable salt thereof, of any one of the preceding claims to the individual on a first day and a second day (e.g., the second day being one or more days after the first day).

73. The method of any one of the preceding claims, wherein the individual receives an initial (e.g., intravenous infusion) dose of the compound, or the pharmaceutically acceptable salt thereof, of any one of the preceding claims on the first day (e.g., to acclimate the individual to vasoconstriction before receiving a first subcutaneous treatment dose).

74. The method of any one of the preceding claims, wherein the initial (e.g., intravenous infusion) dose is about 0.01 milligrams (mg) to about 10 mg.

75. The method of any one of the preceding claims, wherein the compound, or the pharmaceutically acceptable salt thereof, of any one of the preceding claims is administered to the individual on the first day (e.g., by intravenous infusion) for a period of about 4 hr to about 8 hr (e.g., about 6 hr).

76. The method of any one of the preceding claims, wherein the initial (e.g., intravenous infusion) dose of the compound, or the pharmaceutically acceptable salt thereof, of any one of the preceding claims is a low dose, such as a dose of about 5 μg/hr to about 15 μg/hr (e.g., about 8 μg/hr).

77. The method of any one of the preceding claims, wherein the method further comprises (subcutaneously) administering the compound, or the pharmaceutically acceptable salt thereof, of any one of the preceding claims to the individual on one or more day after the first day.

78. The method of any one of the preceding claims, wherein the method comprises (subcutaneously) administering the compound, or the pharmaceutically acceptable salt thereof, of any one of the preceding claims to the individual once a day for 4 to 10 days (e.g., after the first day).

79. The method of any one of the preceding claims, wherein the method further comprises (subcutaneously) administering the compound, or the pharmaceutically acceptable salt thereof, of any one of the preceding claims to the individual on consecutive days after the first day.

80. The method of any one of the preceding claims, wherein the method comprises (subcutaneously) administering the compound, or the pharmaceutically acceptable salt thereof, of any one of the preceding claims to the individual on multiple days.

81. The method of any one of the preceding claims, wherein the individual receives repeated subcutaneous injections of the compound, or the pharmaceutically acceptable salt thereof, of any one of the preceding claims.

82. The method of any one of the preceding claims, wherein the method comprises subcutaneously administering the compound, or the pharmaceutically acceptable salt thereof, of any one of the preceding claims to the individual once- or twice-daily (e.g., for two or more consecutive days).

83. The method of any one of the preceding claims, wherein the method comprises administering the compound, or the pharmaceutically acceptable salt thereof, of any one of the preceding claims to the individual by subcutaneous bolus injection.

84. The method of any one of the preceding claims, wherein the method comprises administering the compound, or the pharmaceutically acceptable salt thereof, of any one of the preceding claims to the individual by (e.g., continuous) subcutaneous infusion.

85. The method of any one of the preceding claims, wherein the method comprises (e.g., subcutaneously) administering the compound, or the pharmaceutically acceptable salt thereof, of any one of the preceding claims to the individual in an amount of about 0.01 milligrams (mg)/day to about 100 mg/day (e.g., about 0.01 milligrams (mg)/day to about 10 mg/day (e.g., about 0.01 mg/day to about 1 mg/day)).

86. The method of any one of the preceding claims, wherein the individual has hepatorenal syndrome with HRS-AKI.

87. The method of any one of the preceding claims, wherein the individual has end-stage liver disease (ESLD).

88. The method of any one of the preceding claims, wherein the individual has developed HRS-AKI as a complication of ESLD.

89. The method of any one of the preceding claims, wherein the method further comprises reducing serum creatinine (sCr) (value) in the individual (e.g., compared to a baseline measurement before treatment).

90. The method of any one of the preceding claims, wherein the method comprises administering the compound, or the pharmaceutically acceptable salt thereof, of any one of the preceding claims to the individual at least until the individual has a sCr value of 1.5 milligrams (mg)/deciliters (dL) or less.

91. The method of any one of claims 1, 8, 24, 28-31, 61, and 62, wherein the method comprises any one of the elements of claims 2-7, 9-23, 25-27, 32-60, and 63-90.

92. A pharmaceutical composition comprising an effective amount of a compound, or a pharmaceutically acceptable salt thereof, wherein the compound is a mixed vasopressin receptor 1A (V1AR) agonist-antagonist, the composition being formulated for subcutaneous administration.

93. The composition of claim 92, wherein the compound has a structure represented by Formula I: 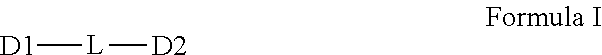
or a pharmaceutically acceptable salt thereof,
wherein:
D1 is a vasopressin receptor 1A (V1AR) agonist;
D2 is a V1AR antagonist; and
L is a linker.

94. The composition according to claim 92 or 93, wherein the compound is Compound 1.

95. A pharmaceutical composition comprising an effective amount of a compound having a structure represented by Formula I: 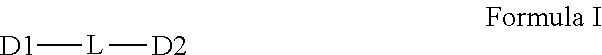
or a pharmaceutically acceptable salt thereof,
wherein:
D1 is a vasopressin receptor 1A (V1AR) agonist;
D2 is a V1AR antagonist; and
L is a linker,
the composition being formulated for subcutaneous administration.

96. A pharmaceutical composition comprising an effective amount of Compound 1, or a pharmaceutically acceptable salt thereof, the composition being formulated for subcutaneous administration.

97. The composition of any one of the preceding claims, wherein the composition is suitable for systemic delivery of an active agent, such as Compound 1.

98. A subcutaneous formulation comprising a compound having a structure represented by Formula I, or a pharmaceutically acceptable salt thereof: 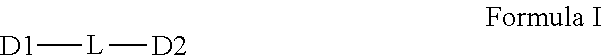
or a pharmaceutically acceptable salt thereof,
wherein:
D1 is a vasopressin receptor 1A (V1AR) agonist;
D2 is a V1AR antagonist; and
L is a linker,
wherein less than 50% of the compound of Formula I degrades (e.g., subcutaneously).

99. A subcutaneous formulation comprising a compound having a structure represented by Formula I, or a pharmaceutically acceptable salt thereof: 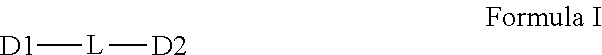
or a pharmaceutically acceptable salt thereof,
wherein:
D1 is a vasopressin receptor 1A (V1AR) agonist;
D2 is a V1AR antagonist; and
L is a linker,
the formulation having a concentration of the compound of Formula I of about 0.1 mg/mL to about 100 mg/mL.

100. A subcutaneous formulation comprising:
a. a compound having a structure represented by Formula I, or a pharmaceutically acceptable salt thereof: 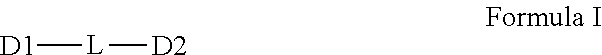
wherein:
D1 is a vasopressin receptor 1A (V1AR) agonist;
D2 is a V1AR antagonist; and
L is a linker, and
b. a buffering agent at a concentration of about 1 millimolar (mM) to about 1 M.

101. The subcutaneous formulation of claims 98-100, further comprising a preservative.

102. A subcutaneous formulation comprising:
a. a compound having a structure represented by Formula I, or a pharmaceutically acceptable salt thereof: 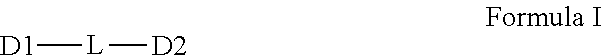
wherein:
D1 is a vasopressin receptor 1A (V1AR) agonist;
D2 is a V1AR antagonist; and
L is a linker, and
b. a preservative.

103. A subcutaneous formulation comprising:
a. a compound having a structure represented by Formula I, or a pharmaceutically acceptable salt thereof: 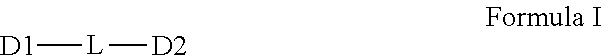
wherein:
D1 is a vasopressin receptor 1A (V1AR) agonist;
D2 is a V1AR antagonist; and
L is a linker, and
b. a solubilizing agent.

104. The subcutaneous formulation of claims 98-103, further comprising preservative (e.g., m-cresol) at a concentration of about 1 mg/mL to about 100 mg/mL.

105. The subcutaneous formulation of claims 98-104, further comprising solubilizing agent (e.g., cyclodextrin) in an amount of about 1 mg/mL to about 250 mg/mL (e.g., about 60-80 mg/mL).

106. The subcutaneous formulation of claims 98-105, wherein the compound of Formula I is less susceptible to degradation, such as in the subcutaneous layer of an individual subcutaneously administered the formulation.

107. The subcutaneous formulation of claims 98-106, wherein less than 50% of the compound of Formula I degrades (e.g., in a vial and/or subcutaneously), such as over a period of about one or two days.

108. The subcutaneous formulation of claims 98-107, wherein the compound of Formula I is present in the formulation at a concentration of about 0.1 mg/mL to about 100 mg/mL.

109. The subcutaneous formulation of claims 98-108, wherein the compound of Formula I is present in the formulation at a concentration of about 1 mg/mL to about 50 mg/mL.

110. The subcutaneous formulation of claims 98-108, further comprising a buffering agent at a concentration of about 1 millimolar (mM) to about 1M.

111. The subcutaneous formulation of claims 98-110, further comprising a buffering agent having a pKa of about 3.0 to about 6.0, such as at 25° C.

112. The subcutaneous formulation according to claim 110 or 111, wherein the buffering agent is selected from the group consisting of acetate, citrate, succinate, and phosphate.

113. The subcutaneous formulation of claims 98-112, having a pH sufficient to inhibit (e.g., deactivate or inactivate) a protease (e.g., trypsin), such as in subcutaneous layer of an individual subcutaneously administered the formulation.

114. The subcutaneous formulation of claims 98-113, having a pH of about 4 to about 5 (e.g., about 4.5).

115. The subcutaneous formulation of claims 98-114, having an ionic strength of about 5 mM to about 200 mM (e.g., about 10 mM to about 100 mM).

116. The subcutaneous formulation of claims 98-115, wherein the pH of the subcutaneous formulation does not (substantially) change when administered subcutaneously to an individual (e.g., by subcutaneous (bolus) injection or subcutaneous infusion).

117. A system for treating end-stage liver disease (ESLD), the system comprising:
(a) a composition comprising a compound of Formula I, or a pharmaceutically acceptable salt thereof; and
(b) a device configured to provide subcutaneous infusion of the composition to an individual when the device is positioned on the skin of the individual.

118. The system of claim 117, wherein the system comprises an adhesive body for (e.g., reversibly) affixing the (subcutaneous infusion) device to the surface of the skin of the individual.

119. The system according to claim 117 or 118, wherein the system comprises a chamber body and a hollow tube body, the composition being configured within the chamber body.

120. The system of any one of claims 117-119, wherein the hollow tube body comprises a first opening and a second opening.

121. The system of any one of claims 117-120, wherein the first opening is in fluid contact with the chamber body.

122. The system of any one of claims 117-121, wherein the second opening is configured subcutaneously within the individual after affixing the subcutaneous infusion device to the skin of the individual.

123. The system of any one of claims 117-122, wherein the (subcutaneous infusion) device further comprises a pump configured to subcutaneously infuse the composition into the individual at a constant or varying rate.

124. The system of any one of claims 117-123, wherein the system is configured to (continuously) provide the composition to the individual over a period of about 24 hours or more.

125. The system of any one of claims 117-124, wherein the device is configured to receive a vial and/or a cartridge of the composition.

126. The system of any one of claims 117-125, wherein the device is a subcutaneous infusion device (e.g., pump).

127. The system of any one of claims 117-126, wherein the composition is the composition or formulation of any one of the preceding claims.
